SEQUENCE LISTING (101 sequences)

SEQ ID NO: 1 - protein, 546 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 2 - protein, 534 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKIPCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTNKVDISSQISSMNQSLQQSKDYIKEAQRLLDTVNPSMKQIEDKIEEILSKIYHIENEIARIKKLIGEGGSLVPRGSGGSEPEA

SEQ ID NO: 3 - protein, 491 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKIPCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTNKVDISSQISSMNQSLQQSKDYIKEAQRLLDTVGGSEPEA

SEQ ID NO: 4 - protein, 491 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKIPCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTNKVDISSQISSMNQSLQQVKDYIKEAQRLLDTVGGSEPEA

SEQ ID NO: 5 - protein, 491 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKIPCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTNKVDISSQISSMNQSLQQSKDYIKEVQRLLDTVGGSEPEA

SEQ ID NO: 6 - protein, 491 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKIPCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTNKVDISSQISSMNQSLQQVKDYIKEVQRLLDTVGGSEPEA

SEQ ID NO: 7 - protein, 546 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQVKDYIKEAQRLLDTVNPSLISMLSMIILYVLSIASLCIGLITFISFIIVEKKRNTYSRLEDRRVRPTSSGDLYYIGT

SEQ ID NO: 8 - protein, 546 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEVQRLLDTVNPSLISMLSMIILYVLSIASLCIGLITFISFIIVEKKRNTYSRLEDRRVRPTSSGDLYYIGT

SEQ ID NO: 9 - protein, 546 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQVKDYIKEVQRLLDTVNPSLISMLSMIILYVLSIASLCIGLITFISFIIVEKKRNTYSRLEDRRVRPTSSGDLYYIGT

SEQ ID NO: 10 - protein, 491 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVGGSEPEA

SEQ ID NO: 11 - protein, 491 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQVKDYIKEAQRLLDTVGGSEPEA

SEQ ID NO: 12 - protein, 491 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEVQRLLDTVGGSEPEA

SEQ ID NO: 13 - protein, 491 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQVKDYIKEVQRLLDTVGGSEPEA

SEQ ID NO: 14 - protein, 491 aa, synthetic construct
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLDSIGGSEPEA

SEQ ID NO: 15 - protein, 494 aa, synthetic construct
MQSSEILILAYSSLLLSSSLCQIPVDKLSNVGVIINEGKLLKIPGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGPPIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 16 - protein, 491 aa, synthetic construct
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEVKEWIRRVNQKLDSIGGSEPEA

SEQ ID NO: 17 - protein, 494 aa, synthetic construct
MQSSEILILAYSSLLLSSSLCQIPVDKLSNVGVIINEGKLLKIPGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 18 - protein, 494 aa, synthetic construct
MQSSEILILAYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGPQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 19 - protein, 494 aa, synthetic construct
MQSSEILILAYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEPIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKVRAIISAVGSGEPEA

SEQ ID NO: 20 - protein, 494 aa, synthetic construct
MQSSEILILAYSSLLLSSSLCQIPVDKLSNVGVIINEGKLLKIPGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEESKTELMKARAIISAVGSGEPEA

SEQ ID NO: 21 - protein, 494 aa, synthetic construct
MQSSEILILAYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGPQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEESKTELMKARAIISAVGSGEPEA

SEQ ID NO: 22 - protein, 494 aa, synthetic construct
MQSSEILILAYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEPIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEESKTELMKARAIISAVGSGEPEA

SEQ ID NO: 23 - protein, 494 aa, synthetic construct
MQSSEILILAYSSLLLSSSLCQIPVDKLSNVGVIINEGKLLKIPGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGPPIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEESKTELMKARAIISAVGSGEPEA

SEQ ID NO: 24 - protein, 494 aa, synthetic construct
MQSSEILILAYSSLLLSSSLCQIPVDKLSNVGVIINEGKLLKIPGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGPPIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEAKTELMKVRAIISAVGSGEPEA

SEQ ID NO: 25 - protein, 494 aa, synthetic construct
MQSSEILILAYSSLLLSSSLCQIPVDKLSNVGVIINEGKLLKIPGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGPPIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEVKTELMKARAIISAVGSGEPEA

SEQ ID NO: 26 - protein, 494 aa, synthetic construct
MQSSEILILAYSSLLLSSSLCQIPVDKLSNVGVIINEGKLLKIPGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGPPIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIISKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEEAKTELMKARAIISAVGSGEPEA

SEQ ID NO: 27 - protein, 1318 aa, synthetic construct
MKLFLILLVLPLASCFFTCNSNANLSMLQLGVPDNSSTIVTGLLPTHWICANQSTSVYSANGFFYIDVGNHRSAFALHTGYYDVNQYYIYVTNEIGLNASVTLKICKFGINTTFDFLSNSSSSFDCIVNLLFTEQLGAPLGITISGETVRLHLYNVTRTFYVPAAYKLTKLSVKCYFNYSCVFSVVNATVTVNVTTHNGRVVNYTVCDDCNGYTDNIFSVQQDGRIPNGFPFNNWFLLTNDSTLVDGVSRLYQPLRLTCLWPVPGLKSSTGFVYFNATGSDVNCNGYQHNSVADVMRYNLNFSANSVDNLKSGVIVFKTLQYDVLFYCSNSSSGVLDTTIPFGPSSQPYYCFINSTINTTHVSTFVGVLPPTVREIVVARTGQFYINGFKYFDLGFIEAVNFNVTTASATDFWTVAFATFVDVLVNVSATKIQNLLYCDSPFEKLQCEHLQFGLQDGFYSANFLDDNVLPETYVALPIYYQHTDINFTATASFGGSCYVCKPHQVNISLNGNTSVCVRTSHFSIRYIYNRVKSGSPGDSSWHIYLKSGTCPFSFSKLNNFQKFKTICFSTVAVPGSCNFPLEATWHYTSYTIVGALYVTWSEGNSITGVPYPVSGIREFSNLVLNNCTKYNIYDYVGTGIIRSSNQSLAGGITYVSNSGNLLGFKNVSTGNIFIVTPCNQPDQVAVYQQSIIGAMTAVNESRYGLQNLLQLPNFYYVSNGGNNCTTAVMTYSNFGICADGSLIPVRPRNSSDNGISAIITANLSIPSNWTTSVQVEYLQITSTPIVVDCATYVCNGNPRCKNLLKQYTSACKTIEDALRLSAHLETNDVSSMLTFDSNAFSLANVTSFGDYNLSSVLPQRNIHSSRIAGRSALEDLLFSKVVTSGLGTVDVDYKSCTKGLSIADLACAQYYNGIMVLPGVADAERMAMYTGSLIGGMVLGGLTSAAAIPFSLALQARLNYVALQTDVLQENQKILAASFNKAINNIVASFSSVNDPITQTAEAIHTVTIALNKIQDVVNQQGSALNHLTSQLRHNFQAISNSIQAIYDRLDPIQADQQVDRLITGRLAALNAFVSQVLNKYTEVRSSRRLAQQKINECVKSQSNRYGFCGNGTHIFSIVNSAPDGLLFLHTVLLPTDYKNVKAWSGICVDGIYGYVLRQPNLVLYSDNGVFRVTSRVMFQPRLPVLSDFVQIYNCNVTFVNISRVELHTVIPDYVDVNKTLQEFAQNLPKYVKPNFDLTPFNLTYLNLSSELKQLEAKTASLFQTTVELQGLIDQINSTYVDLEDKIEEILSKIYHIENEIARIKKLIGEAGSGEPEA

SEQ ID NO: 28 - protein, 1290 aa, synthetic construct
MKLFLILLVLPLASCFFTCNSNANLSMLQLGVPDNSSTIVTGLLPTHWICANQSTSVYSANGFFYIDVGNHRSAFALHTGYYDVNQYYIYVTNEIGLNASVTLKICKFGINTTFDFLSNSSSSFDCIVNLLFTEQLGAPLGITISGETVRLHLYNVTRTFYVPAAYKLTKLSVKCYFNYSCVFSVVNATVTVNVTTHNGRVVNYTVCDDCNGYTDNIFSVQQDGRIPNGFPFNNWFLLTNDSTLVDGVSRLYQPLRLTCLWPVPGLKSSTGFVYFNATGSDVNCNGYQHNSVADVMRYNLNFSANSVDNLKSGVIVFKTLQYDVLFYCSNSSSGVLDTTIPFGPSSQPYYCFINSTINTTHVSTFVGVLPPTVREIVVARTGQFYINGFKYFDLGFIEAVNFNVTTASATDFWTVAFATFVDVLVNVSATKIQNLLYCDSPFEKLQCEHLQFGLQDGFYSANFLDDNVLPETYVALPIYYQHTDINFTATASFGGSCYVCKPHQVNISLNGNTSVCVRTSHFSIRYIYNRVKSGSPGDSSWHIYLKSGTCPFSFSKLNNFQKFKTICFSTVAVPGSCNFPLEATWHYTSYTIVGALYVTWSEGNSITGVPYPVSGIREFSNLVLNNCTKYNIYDYVGTGIIRSSNQSLAGGITYVSNSGNLLGFKNVSTGNIFIVTPCNQPDQVAVYQQSIIGAMTAVNESRYGLQNLLQLPNFYYVSNGGNNCTTAVMTYSNFGICADGSLIPVRPRNSSDNGISAIITANLSIPSNWTTSVQVEYLQITSTPIVVDCATYVCNGNPRCKNLLKQYTSACKTIEDALRLSAHLETNDVSSMLTFDSNAFSLANVTSFGDYNLSSVLPQRNIHSSRIAGRSALEDLLFSKVVTSGLGTVDVDYKSCTKGLSIADLACAQYYNGIMVLPGVADAERMAMYTGSLIGGMVLGGLTSAAAIPFSLALQARLNYVALQTDVLQENQKILAASFNKAINNIVASFSSVNDPITQTAEAIHTVTIALNKIQDVVNQQGSALNHLTSQLRHNFQAISNSIQAIYDRLDPIQADQQVDRLITGRLAALNAFVSQVLNKYTEVRSSRRLAQQKINECVKSQSNRYGFCGNGTHIFSIVNSAPDGLLFLHTVLLPTDYKNVKAWSGICVDGIYGYVLRQPNLVLYSDNGVFRVTSRVMFQPRLPVLSDFVQIYNCNVTFVNISRVELHTVIPDYVDVNKTLQEFAQNLPKYVKPNFDLTPFNLTYLNLSSELKQLEAKVASLFQTTVELQGLIDQINSTYVDLGSGEPEA

SEQ ID NO: 29 - protein, 1290 aa, synthetic construct
MKLFLILLVLPLASCFFTCNSNANLSMLQLGVPDNSSTIVTGLLPTHWICANQSTSVYSANGFFYIDVGNHRSAFALHTGYYDVNQYYIYVTNEIGLNASVTLKICKFGINTTFDFLSNSSSSFDCIVNLLFTEQLGAPLGITISGETVRLHLYNVTRTFYVPAAYKLTKLSVKCYFNYSCVFSVVNATVTVNVTTHNGRVVNYTVCDDCNGYTDNIFSVQQDGRIPNGFPFNNWFLLTNDSTLVDGVSRLYQPLRLTCLWPVPGLKSSTGFVYFNATGSDVNCNGYQHNSVADVMRYNLNFSANSVDNLKSGVIVFKTLQYDVLFYCSNSSSGVLDTTIPFGPSSQPYYCFINSTINTTHVSTFVGVLPPTVREIVVARTGQFYINGFKYFDLGFIEAVNFNVTTASATDFWTVAFATFVDVLVNVSATKIQNLLYCDSPFEKLQCEHLQFGLQDGFYSANFLDDNVLPETYVALPIYYQHTDINFTATASFGGSCYVCKPHQVNISLNGNTSVCVRTSHFSIRYIYNRVKSGSPGDSSWHIYLKSGTCPFSFSKLNNFQKFKTICFSTVAVPGSCNFPLEATWHYTSYTIVGALYVTWSEGNSITGVPYPVSGIREFSNLVLNNCTKYNIYDYVGTGIIRSSNQSLAGGITYVSNSGNLLGFKNVSTGNIFIVTPCNQPDQVAVYQQSIIGAMTAVNESRYGLQNLLQLPNFYYVSNGGNNCTTAVMTYSNFGICADGSLIPVRPRNSSDNGISAIITANLSIPSNWTTSVQVEYLQITSTPIVVDCATYVCNGNPRCKNLLKQYTSACKTIEDALRLSAHLETNDVSSMLTFDSNAFSLANVTSFGDYNLSSVLPQRNIHSSRIAGRSALEDLLFSKVVTSGLGTVDVDYKSCTKGLSIADLACAQYYNGIMVLPGVADAERMAMYTGSLIGGMVLGGLTSAAAIPFSLALQARLNYVALQTDVLQENQKILAASFNKAINNIVASFSSVNDPITQTAEAIHTVTIALNKIQDVVNQQGSALNHLTSQLRHNFQAISNSIQAIYDRLDPIQADQQVDRLITGRLAALNAFVSQVLNKYTEVRSSRRLAQQKINECVKSQSNRYGFCGNGTHIFSIVNSAPDGLLFLHTVLLPTDYKNVKAWSGICVDGIYGYVLRQPNLVLYSDNGVFRVTSRVMFQPRLPVLSDFVQIYNCNVTFVNISRVELHTVIPDYVDVNKTLQEFAQNLPKYVKPNFDLTPFNLTYLNLSSELKQLEAKLASLFQTTVELQGLIDQINSTYVDLGSGEPEA

SEQ ID NO: 30 - protein, 1290 aa, synthetic construct
MKLFLILLVLPLASCFFTCNSNANLSMLQLGVPDNSSTIVTGLLPTHWICANQSTSVYSANGFFYIDVGNHRSAFALHTGYYDVNQYYIYVTNEIGLNASVTLKICKFGINTTFDFLSNSSSSFDCIVNLLFTEQLGAPLGITISGETVRLHLYNVTRTFYVPAAYKLTKLSVKCYFNYSCVFSVVNATVTVNVTTHNGRVVNYTVCDDCNGYTDNIFSVQQDGRIPNGFPFNNWFLLTNDSTLVDGVSRLYQPLRLTCLWPVPGLKSSTGFVYFNATGSDVNCNGYQHNSVADVMRYNLNFSANSVDNLKSGVIVFKTLQYDVLFYCSNSSSGVLDTTIPFGPSSQPYYCFINSTINTTHVSTFVGVLPPTVREIVVARTGQFYINGFKYFDLGFIEAVNFNVTTASATDFWTVAFATFVDVLVNVSATKIQNLLYCDSPFEKLQCEHLQFGLQDGFYSANFLDDNVLPETYVALPIYYQHTDINFTATASFGGSCYVCKPHQVNISLNGNTSVCVRTSHFSIRYIYNRVKSGSPGDSSWHIYLKSGTCPFSFSKLNNFQKFKTICFSTVAVPGSCNFPLEATWHYTSYTIVGALYVTWSEGNSITGVPYPVSGIREFSNLVLNNCTKYNIYDYVGTGIIRSSNQSLAGGITYVSNSGNLLGFKNVSTGNIFIVTPCNQPDQVAVYQQSIIGAMTAVNESRYGLQNLLQLPNFYYVSNGGNNCTTAVMTYSNFGICADGSLIPVRPRNSSDNGISAIITANLSIPSNWTTSVQVEYLQITSTPIVVDCATYVCNGNPRCKNLLKQYTSACKTIEDALRLSAHLETNDVSSMLTFDSNAFSLANVTSFGDYNLSSVLPQRNIHSSRIAGRSALEDLLFSKVVTSGLGTVDVDYKSCTKGLSIADLACAQYYNGIMVLPGVADAERMAMYTGSLIGGMVLGGLTSAAAIPFSLALQARLNYVALQTDVLQENQKILAASFNKAINNIVASFSSVNDPITQTAEAIHTVTIALNKIQDVVNQQGSALNHLTSQLRHNFQAISNSIQAIYDRLDPIQADQQVDRLITGRLAALNAFVSQVLNKYTEVRSSRRLAQQKINECVKSQSNRYGFCGNGTHIFSIVNSAPDGLLFLHTVLLPTDYKNVKAWSGICVDGIYGYVLRQPNLVLYSDNGVFRVTSRVMFQPRLPVLSDFVQIYNCNVTFVNISRVELHTVIPDYVDVNKTLQEFAQNLPKYVKPNFDLTPFNLTYLNLSSELKQLEAKIASLFQTTVELQGLIDQINSTYVDLGSGEPEA

SEQ ID NO: 31 - protein, 1290 aa, synthetic construct
MKLFLILLVLPLASCFFTCNSNANLSMLQLGVPDNSSTIVTGLLPTHWICANQSTSVYSANGFFYIDVGNHRSAFALHTGYYDVNQYYIYVTNEIGLNASVTLKICKFGINTTFDFLSNSSSSFDCIVNLLFTEQLGAPLGITISGETVRLHLYNVTRTFYVPAAYKLTKLSVKCYFNYSCVFSVVNATVTVNVTTHNGRVVNYTVCDDCNGYTDNIFSVQQDGRIPNGFPFNNWFLLTNDSTLVDGVSRLYQPLRLTCLWPVPGLKSSTGFVYFNATGSDVNCNGYQHNSVADVMRYNLNFSANSVDNLKSGVIVFKTLQYDVLFYCSNSSSGVLDTTIPFGPSSQPYYCFINSTINTTHVSTFVGVLPPTVREIVVARTGQFYINGFKYFDLGFIEAVNFNVTTASATDFWTVAFATFVDVLVNVSATKIQNLLYCDSPFEKLQCEHLQFGLQDGFYSANFLDDNVLPETYVALPIYYQHTDINFTATASFGGSCYVCKPHQVNISLNGNTSVCVRTSHFSIRYIYNRVKSGSPGDSSWHIYLKSGTCPFSFSKLNNFQKFKTICFSTVAVPGSCNFPLEATWHYTSYTIVGALYVTWSEGNSITGVPYPVSGIREFSNLVLNNCTKYNIYDYVGTGIIRSSNQSLAGGITYVSNSGNLLGFKNVSTGNIFIVTPCNQPDQVAVYQQSIIGAMTAVNESRYGLQNLLQLPNFYYVSNGGNNCTTAVMTYSNFGICADGSLIPVRPRNSSDNGISAIITANLSIPSNWTTSVQVEYLQITSTPIVVDCATYVCNGNPRCKNLLKQYTSACKTIEDALRLSAHLETNDVSSMLTFDSNAFSLANVTSFGDYNLSSVLPQRNIHSSRIAGRSALEDLLFSKVVTSGLGTVDVDYKSCTKGLSIADLACAQYYNGIMVLPGVADAERMAMYTGSLIGGMVLGGLTSAAAIPFSLALQARLNYVALQTDVLQENQKILAASFNKAINNIVASFSSVNDPITQTAEAIHTVTIALNKIQDVVNQQGSALNHLTSQLRHNFQAISNSIQAIYDRLDPIQADQQVDRLITGRLAALNAFVSQVLNKYTEVRSSRRLAQQKINECVKSQSNRYGFCGNGTHIFSIVNSAPDGLLFLHTVLLPTDYKNVKAWSGICVDGIYGYVLRQPNLVLYSDNGVFRVTSRVMFQPRLPVLSDFVQIYNCNVTFVNISRVELHTVIPDYVDVNKTLQEFAQNLPKYVKPNFDLTPFNLTYLNLSSELKQLEAKTASLFQTVVELQGLIDQINSTYVDLGSGEPEA

SEQ ID NO: 32 - protein, 1290 aa, synthetic construct
MKLFLILLVLPLASCFFTCNSNANLSMLQLGVPDNSSTIVTGLLPTHWICANQSTSVYSANGFFYIDVGNHRSAFALHTGYYDVNQYYIYVTNEIGLNASVTLKICKFGINTTFDFLSNSSSSFDCIVNLLFTEQLGAPLGITISGETVRLHLYNVTRTFYVPAAYKLTKLSVKCYFNYSCVFSVVNATVTVNVTTHNGRVVNYTVCDDCNGYTDNIFSVQQDGRIPNGFPFNNWFLLTNDSTLVDGVSRLYQPLRLTCLWPVPGLKSSTGFVYFNATGSDVNCNGYQHNSVADVMRYNLNFSANSVDNLKSGVIVFKTLQYDVLFYCSNSSSGVLDTTIPFGPSSQPYYCFINSTINTTHVSTFVGVLPPTVREIVVARTGQFYINGFKYFDLGFIEAVNFNVTTASATDFWTVAFATFVDVLVNVSATKIQNLLYCDSPFEKLQCEHLQFGLQDGFYSANFLDDNVLPETYVALPIYYQHTDINFTATASFGGSCYVCKPHQVNISLNGNTSVCVRTSHFSIRYIYNRVKSGSPGDSSWHIYLKSGTCPFSFSKLNNFQKFKTICFSTVAVPGSCNFPLEATWHYTSYTIVGALYVTWSEGNSITGVPYPVSGIREFSNLVLNNCTKYNIYDYVGTGIIRSSNQSLAGGITYVSNSGNLLGFKNVSTGNIFIVTPCNQPDQVAVYQQSIIGAMTAVNESRYGLQNLLQLPNFYYVSNGGNNCTTAVMTYSNFGICADGSLIPVRPRNSSDNGISAIITANLSIPSNWTTSVQVEYLQITSTPIVVDCATYVCNGNPRCKNLLKQYTSACKTIEDALRLSAHLETNDVSSMLTFDSNAFSLANVTSFGDYNLSSVLPQRNIHSSRIAGRSALEDLLFSKVVTSGLGTVDVDYKSCTKGLSIADLACAQYYNGIMVLPGVADAERMAMYTGSLIGGMVLGGLTSAAAIPFSLALQARLNYVALQTDVLQENQKILAASFNKAINNIVASFSSVNDPITQTAEAIHTVTIALNKIQDVVNQQGSALNHLTSQLRHNFQAISNSIQAIYDRLDPIQADQQVDRLITGRLAALNAFVSQVLNKYTEVRSSRRLAQQKINECVKSQSNRYGFCGNGTHIFSIVNSAPDGLLFLHTVLLPTDYKNVKAWSGICVDGIYGYVLRQPNLVLYSDNGVFRVTSRVMFQPRLPVLSDFVQIYNCNVTFVNISRVELHTVIPDYVDVNKTLQEFAQNLPKYVKPNFDLTPFNLTYLNLSSELKQLEAKTASLFQTLVELQGLIDQINSTYVDLGSGEPEA

SEQ ID NO: 33 - protein, 1290 aa, synthetic construct
MKLFLILLVLPLASCFFTCNSNANLSMLQLGVPDNSSTIVTGLLPTHWICANQSTSVYSANGFFYIDVGNHRSAFALHTGYYDVNQYYIYVTNEIGLNASVTLKICKFGINTTFDFLSNSSSSFDCIVNLLFTEQLGAPLGITISGETVRLHLYNVTRTFYVPAAYKLTKLSVKCYFNYSCVFSVVNATVTVNVTTHNGRVVNYTVCDDCNGYTDNIFSVQQDGRIPNGFPFNNWFLLTNDSTLVDGVSRLYQPLRLTCLWPVPGLKSSTGFVYFNATGSDVNCNGYQHNSVADVMRYNLNFSANSVDNLKSGVIVFKTLQYDVLFYCSNSSSGVLDTTIPFGPSSQPYYCFINSTINTTHVSTFVGVLPPTVREIVVARTGQFYINGFKYFDLGFIEAVNFNVTTASATDFWTVAFATFVDVLVNVSATKIQNLLYCDSPFEKLQCEHLQFGLQDGFYSANFLDDNVLPETYVALPIYYQHTDINFTATASFGGSCYVCKPHQVNISLNGNTSVCVRTSHFSIRYIYNRVKSGSPGDSSWHIYLKSGTCPFSFSKLNNFQKFKTICFSTVAVPGSCNFPLEATWHYTSYTIVGALYVTWSEGNSITGVPYPVSGIREFSNLVLNNCTKYNIYDYVGTGIIRSSNQSLAGGITYVSNSGNLLGFKNVSTGNIFIVTPCNQPDQVAVYQQSIIGAMTAVNESRYGLQNLLQLPNFYYVSNGGNNCTTAVMTYSNFGICADGSLIPVRPRNSSDNGISAIITANLSIPSNWTTSVQVEYLQITSTPIVVDCATYVCNGNPRCKNLLKQYTSACKTIEDALRLSAHLETNDVSSMLTFDSNAFSLANVTSFGDYNLSSVLPQRNIHSSRIAGRSALEDLLFSKVVTSGLGTVDVDYKSCTKGLSIADLACAQYYNGIMVLPGVADAERMAMYTGSLIGGMVLGGLTSAAAIPFSLALQARLNYVALQTDVLQENQKILAASFNKAINNIVASFSSVNDPITQTAEAIHTVTIALNKIQDVVNQQGSALNHLTSQLRHNFQAISNSIQAIYDRLDPIQADQQVDRLITGRLAALNAFVSQVLNKYTEVRSSRRLAQQKINECVKSQSNRYGFCGNGTHIFSIVNSAPDGLLFLHTVLLPTDYKNVKAWSGICVDGIYGYVLRQPNLVLYSDNGVFRVTSRVMFQPRLPVLSDFVQIYNCNVTFVNISRVELHTVIPDYVDVNKTLQEFAQNLPKYVKPNFDLTPFNLTYLNLSSELKQLEAKTASLFQTIVELQGLIDQINSTYVDLGSGEPEA

SEQ ID NO: 34 - protein, 1290 aa, synthetic construct
MKLFLILLVLPLASCFFTCNSNANLSMLQLGVPDNSSTIVTGLLPTHWICANQSTSVYSANGFFYIDVGNHRSAFALHTGYYDVNQYYIYVTNEIGLNASVTLKICKFGINTTFDFLSNSSSSFDCIVNLLFTEQLGAPLGITISGETVRLHLYNVTRTFYVPAAYKLTKLSVKCYFNYSCVFSVVNATVTVNVTTHNGRVVNYTVCDDCNGYTDNIFSVQQDGRIPNGFPFNNWFLLTNDSTLVDGVSRLYQPLRLTCLWPVPGLKSSTGFVYFNATGSDVNCNGYQHNSVADVMRYNLNFSANSVDNLKSGVIVFKTLQYDVLFYCSNSSSGVLDTTIPFGPSSQPYYCFINSTINTTHVSTFVGVLPPTVREIVVARTGQFYINGFKYFDLGFIEAVNFNVTTASATDFWTVAFATFVDVLVNVSATKIQNLLYCDSPFEKLQCEHLQFGLQDGFYSANFLDDNVLPETYVALPIYYQHTDINFTATASFGGSCYVCKPHQVNISLNGNTSVCVRTSHFSIRYIYNRVKSGSPGDSSWHIYLKSGTCPFSFSKLNNFQKFKTICFSTVAVPGSCNFPLEATWHYTSYTIVGALYVTWSEGNSITGVPYPVSGIREFSNLVLNNCTKYNIYDYVGTGIIRSSNQSLAGGITYVSNSGNLLGFKNVSTGNIFIVTPCNQPDQVAVYQQSIIGAMTAVNESRYGLQNLLQLPNFYYVSNGGNNCTTAVMTYSNFGICADGSLIPVRPRNSSDNGISAIITANLSIPSNWTTSVQVEYLQITSTPIVVDCATYVCNGNPRCKNLLKQYTSACKTIEDALRLSAHLETNDVSSMLTFDSNAFSLANVTSFGDYNLSSVLPQRNIHSSRIAGRSALEDLLFSKVVTSGLGTVDVDYKSCTKGLSIADLACAQYYNGIMVLPGVADAERMAMYTGSLIGGMVLGGLTSAAAIPFSLALQARLNYVALQTDVLQENQKILAASFNKAINNIVASFSSVNDPITQTAEAIHTVTIALNKIQDVVNQQGSALNHLTSQLRHNFQAISNSIQAIYDRLDPIQADQQVDRLITGRLAALNAFVSQVLNKYTEVRSSRRLAQQKINECVKSQSNRYGFCGNGTHIFSIVNSAPDGLLFLHTVLLPTDYKNVKAWSGICVDGIYGYVLRQPNLVLYSDNGVFRVTSRVMFQPRLPVLSDFVQIYNCNVTFVNISRVELHTVIPDYVDVNKTLQEFAQNLPKYVKPNFDLTPFNLTYLNLSSELKQLEAKVASLFQTVVELQGLIDQINSTYVDLGSGEPEA

SEQ ID NO: 35 - protein, 1290 aa, synthetic construct
MKLFLILLVLPLASCFFTCNSNANLSMLQLGVPDNSSTIVTGLLPTHWICANQSTSVYSANGFFYIDVGNHRSAFALHTGYYDVNQYYIYVTNEIGLNASVTLKICKFGINTTFDFLSNSSSSFDCIVNLLFTEQLGAPLGITISGETVRLHLYNVTRTFYVPAAYKLTKLSVKCYFNYSCVFSVVNATVTVNVTTHNGRVVNYTVCDDCNGYTDNIFSVQQDGRIPNGFPFNNWFLLTNDSTLVDGVSRLYQPLRLTCLWPVPGLKSSTGFVYFNATGSDVNCNGYQHNSVADVMRYNLNFSANSVDNLKSGVIVFKTLQYDVLFYCSNSSSGVLDTTIPFGPSSQPYYCFINSTINTTHVSTFVGVLPPTVREIVVARTGQFYINGFKYFDLGFIEAVNFNVTTASATDFWTVAFATFVDVLVNVSATKIQNLLYCDSPFEKLQCEHLQFGLQDGFYSANFLDDNVLPETYVALPIYYQHTDINFTATASFGGSCYVCKPHQVNISLNGNTSVCVRTSHFSIRYIYNRVKSGSPGDSSWHIYLKSGTCPFSFSKLNNFQKFKTICFSTVAVPGSCNFPLEATWHYTSYTIVGALYVTWSEGNSITGVPYPVSGIREFSNLVLNNCTKYNIYDYVGTGIIRSSNQSLAGGITYVSNSGNLLGFKNVSTGNIFIVTPCNQPDQVAVYQQSIIGAMTAVNESRYGLQNLLQLPNFYYVSNGGNNCTTAVMTYSNFGICADGSLIPVRPRNSSDNGISAIITANLSIPSNWTTSVQVEYLQITSTPIVVDCATYVCNGNPRCKNLLKQYTSACKTIEDALRLSAHLETNDVSSMLTFDSNAFSLANVTSFGDYNLSSVLPQRNIHSSRIAGRSALEDLLFSKVVTSGLGTVDVDYKSCTKGLSIADLACAQYYNGIMVLPGVADAERMAMYTGSLIGGMVLGGLTSAAAIPFSLALQARLNYVALQTDVLQENQKILAASFNKAINNIVASFSSVNDPITQTAEAIHTVTIALNKIQDVVNQQGSALNHLTSQLRHNFQAISNSIQAIYDRLDPIQADQQVDRLITGRLAALNAFVSQVLNKYTEVRSSRRLAQQKINECVKSQSNRYGFCGNGTHIFSIVNSAPDGLLFLHTVLLPTDYKNVKAWSGICVDGIYGYVLRQPNLVLYSDNGVFRVTSRVMFQPRLPVLSDFVQIYNCNVTFVNISRVELHTVIPDYVDVNKTLQEFAQNLPKYVKPNFDLTPFNLTYLNLSSELKQLEAKTASLFQTTVELQGLIDQINSTYVDLGSGEPEA

SEQ ID NO: 36 - protein, 565 aa, synthetic construct
MATYIQRVQCISALLSVVLTTLVSCQIPRDRLSNIGVIVDEGKSLKIAGSHESRYIVLSLVPGIDLENGCGTAQVIQYKSLLNRLLIPLRDALDLQEALITVTNDTMTGADVPQSRFFGAVIGTIALGVATSAQITAGIALAEAREAKRDIALIKESMTKTHKSIELLQNAVGEQILALKTLQDFVNDEIKPAISELGCETAALRLGIKLTQHYSELLTAFGSNFGTIGEKSLTLQALSSLYSANITEIMTTIRTGQSNIYDVIYTEQIKGTVIDVDLERYMVTLSVKIPILSEVPGVLIHKASSISYNIDGEEWYVTVPSHILSRASFLGGANIADCVESRLTYICPRDPAQLIPDSQQKCILGDTTRCPVTKVVDNIIPKFAFVNGGVVANCIASTCTCGTGRRPISQDRSKGVVFLTHDNCGLIGVNGIELYANRKGHDATWGVQNLTVGPAIAIRPVDISLNLAAATDFLQDSRAELEKARKILSEVGRWYNSGATLITIIVVMIVVLVVIIVIVIVLYRLRRSMLMSNPAGRISRDTYTLEPKIRHMYTNGGFDAMTEKR

SEQ ID NO: 37 - protein, 551 aa, synthetic construct
MHHLHPMIVCIFVMYTGIVGSDAIAGDQLLNIGVIQSKIRSLMYYTDGGASFIVVKLLPNLPPSNGTCNITSLDAYNVTLFKLLTPLIENLSKISAVTDTKTRQERFAGVVIGLAALGVATAAQITAAVAIVKANANAAAINNLASSIQSTNKAVSDVIDASRTIATAVQAIQDHINGAIVNGITSASCRAHDALIGSILNLYLTELTTIFHNQITNPALTPLSIQALRILLGSTLPIVIESKLNTNLNTAELLSSGLLTGQIISISPMYMQMLIQINVPTFIMQPGAKVIDLIAISANHKLQEVVVQVPNRILEYANELQNYPANDCVVTPNSVFCRYNEGSPIPESQYQCLRGNLNSCTFTPIIGNFLKRFAFANGVLYANCKSLLCKCADPPHVVSQDDTQGISIIDIKRCSEMMLDTFSFRITSTFNATYVTDFSMINANIVHLSPLDLSNQINSINKSLKSAEDWIADSNFFANQARTAKTLYSLSAIALILSVITLVVVGLLIAYIIKLVSQIHQFRALAATTMFHRENPAFFSKNNHGNIYGIS

SEQ ID NO: 38 - protein, 543 aa, synthetic construct
MGVKGLSLVMIGLLISPITNLDITHLMNLGTVPTAIRSLVYYTYSKPSYLTVDLIPNLKNLDPKCNYSSLNYYNKTALSLIQPIADNINRLTKPITSSEIQSRFFGAVIGTVALGVATAAQVTAAIGLAKAQENARLILTLKKAAKETNDAVRDLIKSNKIVARMISAIQNQINTIIQPAIDRINCQIKDLQVANILNLYLTEITTVFHNQLTNPALESISIQALKSLLGSTLPEVLSKLDLNNISAASVMASGLIKGQIIAVDIPTMTLVLMVQIPSISPLRQAKIIDLTSITIHTNNQEVQAVVPDRVLEIGSEILGFDGSVCQITKDTVFCPYNDAYILPIQQKRCLQGQTRDCVFTPVAGTFPRRFLTTYGTIVANCRNLVCSCLRPPQIIYQPDENPVTIIDKDLCTTLTLDSITIEIQKSINSTFRREVVLESTQVRSLTPLDLSTDLSQYNQLLKSAEDHIQRSNDYLNSINPSIVNNSAIIILIILCILLILTVTICIIWLKYLTKEVKNVARNQRLNRDADLIYKIPSQIPLPR

SEQ ID NO: 39 - protein, 551 aa, synthetic construct
MGTIIQFLVVSCLLAGAGSLDPAALMQIGVIPTNVRQLMYYTEASSAFIVVKLMPTIDSPISGCNITSISSYNATVTKLLQPIGENLETIRNQLIPTRRRRRFAGVVIGLAALGVATAAQVTAAVALVKANENAAAILNLKNAIQKTNAAVADVVQATQSLGTAVQAVQDHINSVVSPAITAANCKAQDAIIGSILNLYLTELTTIFHNQITNPALSPITIQALRILLGSTLPTVVEKSFNTQISVAELLSSGLLTGQIVGLDLTYMQMVIKIELPTLAVQPATQIIDLATISAFINNQEVMAQLPTRVIVTGSLIQAYPASQCTITPNTVYCRYNDARVLSDDTMACLQGNLTRCTFSPVVGSFLTRFVLFDGIVYANCRSMLCKCMQPAAVILQPSSSPVTVIDMYKCVSLRLDNLRFTITQLANVTYNSTIKLETSQILPIAPLDISQNLAAVNKSLSDALQHLAQSDTYLSAITSATTTSVLSIIAICLGSLGLILIILLSVVVWKLLTIVAANRNRMENFVYHNSAFHHSRSDLSEKNQPATLGTR

SEQ ID NO: 40 - protein, 538 aa, synthetic construct
MKAFLVTCLGFAVFSFSICVNINILQQIGYIKQQVRQLSYYSQSSSSYIVVKLLPNIQPTDNSCEFKSVTQYNKTLSNLLLPIAENINNIASPSPGSRRHKRFAGIAIGIAALGVATAAQVTAAVSLVQAQTNARAIAAMKNSIQATNRAVFEVKEGTQQLAIAVQAIQDHINTIMNTQLNNMSCQILDNQLATSLGLYLTELTTVFQPQLINPALSPISIQALRSLLGSMTPAVVQATLSTSISAAEILSAGLMEGQIVSVLLDEMQMIVKINIPTIVTQSNALVIDFYSISSFINNQESIIQLPDRILEIGNEQWSYPAKNCKLTRHNIFCQYNEAERLSLESKLCLAGNISACVFSPIAGSYMRRFVALDGTIVANCRSLTCLCKSPSYPIYQPDHHAVTTIDLTACQTLSLDGLDFSIVSLSNITYAENLTISLSQTINTQPIDISTELSKVNASLQNAVKYIKESNHQLQSVSVNSKIGAIIIAALVLSILSIIISLLFCCWAYIATKEIRRINFKTNHINTISSSVDDLIRY

SEQ ID NO: 41 - protein, 550 aa, synthetic construct
MGLKVNVSAIFMAVLLTLQTPTGQIHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILRSMKGLSSTSIVYILIAVCLGGLIGIPALICCCRGRCNKKGEQVGMSRPGLKPDLTGTSKSYVRSL

SEQ ID NO: 42 - protein, 546 aa, synthetic construct
MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 43 - protein, 553 aa, synthetic construct
MGSRPFTKNPAPMMLTIRVALVLSCICPANSIDGRPFAAAGIVVTGDKAVNIYTSSQTGSIIVKLLPNLPKDKEACAKAPLDAYNRTLTTLLTPLGDSIRRIQESVTTSGGGRQGRLIGAIIGGVALGVATAAQITAAAALIQAKQNAANILRLKESIAATNEAVHEVTDGLSQLAVAVGKMQQFVNDQFNKTAQELDCIKIAQQVGVELNLYLTELTTVFGPQITSPALNKLTIQALYNLAGGNMDYLLTKLGIGNNQLSSLIGSGLITGNPILYDSQTQLLGIQVTLPSVGNLNNMRATYLETLSVSTTRGFASALVPKVVTQVGSVIEELDTSYCIETDLDLYCTRIVTFPMSPGIYSCLSGNTSACMYSKTEGALTTPYMTIKGSVIANCKMTTCRCVNPPGIISQNYGEAVSLIDKQSCNVLSLGGITLRLSGEFDVTYQKNISIQDSQVIITGNLDISTELGNVNNSISNALNKLEESNRKLDKVNVKLTSTSALITYIVLTIISLVFGILSLILACYLMYKQKAQQKTLLWLGNNTLDQMRATTKM

SEQ ID NO: 44 - protein, 553 aa, synthetic construct
MGSKPSTRILAPLMLITRIMLTLSCIRLTSSLDGRPLAAAGIVVTGDKAVNVYTSSQTGSIIVKLLPNMPRDKEACARAPLEAYNRTLTTLLTPLGDSIRKIQGSVSTSGGRRQKRFIGAVIGSVALGVATAAQITAAAALIQAKQNAANILRLKESIAATNEAVHEVTDGLSQLSVAVGKMQQFVNDQFNNTARELDCIKITQQVGVELNLYLTELTTVFGPQITSPALTQLTIQALYNLAGGNMDYLLTKLGIGNNQLSSLIGSGLITGYPILYDSHTQLLGIQVNLPSVGNLNNMRATYLETLSVSTTKGYASALVPKVVTQVGSVIEELDTSYCIESDLDLYCTRIVTFPMSPGIYSCLSGNTSACMYSKTEGALTTPYMALRGSVIANCKITTCRCTDPPGIISQNYGEAVSLIDRHSCNVLSLDGITLRLSGEFDATYQKNISILDSQVIVTGNLDISTELGNVNNSISNALDRLAESSSKLERVNVRLTSTSALITYIVLTVISLIFGALSLALACYLMYKQKAQQKTLLWLGNNTLDQMRATTRA

SEQ ID NO: 45 - protein, 662 aa, synthetic construct
MHNKIPKKSKPLPHTRQDPLQQQSTKSAETKTSQGRYSITSAQRSTYHGPRTSDRSVHYIMNRTRSCKQTSHRSDNIPPHRDHEGIIHHTPESVTQGASSWFKRRQSNATNAGSQCTWLVLWCIGIASLFLCSKAQIHWNNLSTIGIIGTDSVHYKIMTRPSHQYLVIKLMPNVSLIDNCTKAELGEYEKLLNSVLEPINQALTLMTNNVKPLQSVGSGRRQRRFAGVVLAGAALGVATAAQITAGIALHQSNLNAQAIQSLRTSLEQSNKAIEEIREATQETVIAVQGVQDYVNNELVPAMQHMSCELVGQRLGLKLLRYYTELLSIFGPSLRDPISAEISIQALSYALGGEIHKILEKLGYSGNDMIAILESRGIKTKITHVDLPGKLIILSISYPTLSEVKGVIVHRLEAVSYNIGSQEWYTTVPKYVATNGYLISNFDESSCVFVSESAICSQNSLYPMSPILQQCIRGDTSSCARTWVSGTMGNKFILSKGNIVANCASILCKCYSTSTIINQSPDKLLTFIASDTCPLVEIDGVTIQVGGRQYPDMVYESKVALGPAISLERLDVGTNLGNALKKLDDAKVLIDSSNQILETVKRSSFNFSSLLSVPILICTALALLLLIYCCKRRYRQTFKHNTKVDPTFKPDLTGTSKSYVRSL

SEQ ID NO: 46 - protein, 543 aa, synthetic construct
MNRIKVIIISSLLLSDITIAQIGWDNLTSIGVISTKQYDYKITTLNTNQLMVIKMVPNISSIINCTKLELTKYRELVSGIIRPINESLELMNSYINMRAGSERFIGAVIAGVALGVATAAQITSGIALHNSIMNKKQIQELRKALSATNKAIDEIRIAGERTLIAVQGVQDYINNIIIPMQDKLQCDILSSQLSVALLRYYTNILTEFGPSIRDPVTSTISVQALSQAFNGNLQALLDGLGYSGRDLRDLLESKSITGQIIHADMTDLFLVLRINYPSITEMQGVTIYGLNSITYHIGPEEWYTIMPDFVAVQGFLISNFDERKCSITKSSILCQQNSIYPMSTEMQRCIKGEIRFCPRSKAIGTLVNRFILTEGNLMANCLGIICRCYTSGQVITQDPSKLITIISQEECKEVGVDGIRIMVGPRKLPDITFNARLEIGVPISLSKLDVGTDLAIASAKLNNSKALLEQSDKILYSMSKLDSINSRIIGLILAIMIIFVIIITIIWIIYKKCRNKDNKFSTSIEPLYIPPSYNSPHSVVKSI

SEQ ID NO: 47 - protein, 557 aa, synthetic construct
MQVTTLGLAITLSIASLVTSQVPRDKLANLGIIIKDSKALKIAGSYENRYIVLSLVPTIDNVNGCGSIQIAKYKEMLERLLIPIKDALDLQESLIVIDNETTANTYHPQYRFVGAIIGTIALGVATAAQVTAGVALMEAREAKRDISMLKEAMEKTQNSIEKLQNSAGEQILALKMLQDYVNGEIKPAIEELGCETAALKLGIALTQHYTELTNAFGSNLGSIGEKSLTLQALSSLYKTNITDILTATNLRKTDIYDIIYAEQVKGRVIDVDLRRYMVTISIKIPILSEIPGVLIYEVSSISYNIDGAEWYAAVPDHILSKSAYIGGADVSDCIESGLTYICPRDPAQIIADNQQKCFLGHLDKCPVTKVIDNLVSKFAFINGGVVANCIASTCTCGEERIQVSQDRDKGVTFLTHNNCGLIGVNGVELHANKKGSDATWNVSPIGVGPAVSLRPVDISLQIVAATNFLNSSRKDLMKAKEVLNQVGNLKDLTVITIINIKIIIVLLICVIGLGILYHQLRSALEMRDKMSALNNSSYSLEPRTVQVQVMKPISFTR

SEQ ID NO: 48 - protein, 545 aa, synthetic construct
MALNKNMFSSLFLGYLLVYATTVQSSIHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH

SEQ ID NO: 49 - protein, 553 aa, synthetic construct
MAIKLIVRVAIILVSLSITFGQVDYGRLRKIGVFEKQTMNLKLSISASQRYMVIKTVPNLGTVSSCGDKEMAAYKESIRKLISPMHDMIEYIKGEVVVEAATSVALNGTQVRFFGLVVAIGALGLATSAQITAGIALHNSLENAKAIKGLSDAMKESNQAIQKLQDATAGTVIALNALQDQINTQIVPALNTLGCSVVSNTLGVALTRYYSELVQLFGPSLANPVEAPLSIQAISGAFNGDLKGMIRDYGYSPSDLEDIIRTGAITGRVIDVDMEDLTIILEISLPTLLVMRDTKVVNFGRITYNLNGSEWQTLSPDWIAIRNTLMSGVDLSTCVMSRQNLICKQDPTFAIDHTVSQCLRGEITSCPRGRVVNSIAPRFAIVNGNVLGNCVATTCLCGDPGTPVIQDASSSLTIMSIDKCELVSIDGYNFRPGPPVVNTTFHLSIDDIGPEVSVNPIDISGALGKIEQDLQSSKEHLANSDTILASINPKIINTNAAIGLIVVSSLGLIAAVLALCWLCCLTKSMVGRDSTAFIGGKGPDMGPIMSSLEGMSF

SEQ ID NO: 50 - protein, 552 aa, synthetic construct
MASNDNSVIYHSFLTVILLVVVTEGQIHWGNLSKIGIVGTGSASYKVMTRPSHQYLVIKLMPNVTMIDNCTRTEVAEYRKLLKTVLEPVRNALTVITKNIKPIQSLTTSRRSKRFAGVVLAGVALGVATAAQITAGIALHQSIMNSQSIDNLRTSLEKSNQAIEEIRQASQETVLAVQGVQDFINNELIPSMHQLSCEMLGQKLGLKLLRYYTEILSIFGPSLRDPVSAEISIQALTYALGGDINKVLEKLGYSGADLLAILESRGIKAKITHVDLEGYFIVLSIAYPTLSEVKGVIVHRLEAVSYNLGSQEWYTTLPRYVATNGYLISNFDESSCAFMSEVTICSQNALYPMSPLLQQCLRGSTASCARSLVSGTIGNRFILSKGNLIANCASVLCKCYSTGTIISQDPDKLLTFVAADKCPLVEVDGITIQVGSREYPDSVYVSRIDLGPPISLEKLDVGTNLGNALTKLNNAKELLDSSNQILENVRRGSFGGAMYIGILVCAGALVILCVLVYCCKRYCCSRVQIVPKSTPGLKPDLTGTTKSYVRSL

SEQ ID NO: 51 - protein, 544 aa, synthetic construct
MKVTTLLIILILITSTLCQISFDNLEQVGVMFDKPKFLKITGPASTATMIIKLIPTLGTMESCGTSAVNEYKKTLDTILIPLRDTINKLSTDITVLEGTPKTLSRREKRFVGIAIAVGAVALATSAQITAGIALANTIKNAEAIESLKSSIQASNKAIQKVIDAQGRTVTVINGIQDHINSVINPALNQLGCDVAKNTLAISLTQYFSKLSLLFGPNLRNPVEQPLSVQAIAGLMDGDINAVVSQLGYTPSDLLDLLSTESIVGTVTAVDMVNYMIQIEMSFPQYITIADTKVLEGHRITFNDRGSEWQTQVPSTIAVRDILIAGVDPDGCSVTSTSYICKNDPTYAMSEVLTNCFRGNTQECPRARITSTFATRFAIARSTVIANCVAAVCLCGDPGTPVVQKAEVTLTAMTLDQCSLITVDGLQIKPSKSLANVTANFGNITLGPVVSVGDLDLSAELTKVQSDLKEAQDRLDESNSILQGINNKILTAPTSIALIVVSVIIVLIIIGIISWLAWLTRAVKRSSIRSERVTPNTYNNLGFIK

SEQ ID NO: 52 - protein, 541 aa, synthetic construct
MAFLKSAIICYLLFYPHIVKSSLHYDSLSKVGIIKGLTYNYKIKGSPSTKLMVVKLIPNIDGVRNCTQKQFDEYKNLVKNVLEPVKLALNAMLDNVKSGNNKYRFAGAIMAGVALGVATAATVTAGIALHRSNENAQAIANMKNAIQNTNEAVKQLQLANKQTLAVIDTIRGEINNNIIPVINQLSCDTIGLSVGIKLTQYYSEILTAFGPALQNPVNTRITIQAISSVFNRNFDELLKIMGYTSGDLYEILHSGLIRGNIIDVDVEAGYIALEIEFPNLTLVPNAVVQELMPISYNVDGDEWVTLVPRFVLTRTTLLSNIDTSRCTVTESSVICDNDYALPMSYELIGCLQGDTSKCAREKVVSSYVPRFALSDGLVYANCLNTICRCMDTDTPISQSLGTTVSLLDNKKCLVYQVGDILISVGSYLGEGEYSADNVELGPPVVIDKIDIGNQLAGINQTLQNAEDYIEKSEEFLKGINPSIITLGSMAVLYIFMIVIAVISIIALVLSIKLTVKGNVVRQQFAYTQHVPSMENVNYVSH

SEQ ID NO: 53 - protein, 1356 aa, synthetic construct
MKLFLILLVLPLASCFFTCNSNANLSMLQLGVPDNSSTIVTGLLPTHWICANQSTSVYSANGFFYIDVGNHRSAFALHTGYYDVNQYYIYVTNEIGLNASVTLKICKFGINTTFDFLSNSSSSFDCIVNLLFTEQLGAPLGITISGETVRLHLYNVTRTFYVPAAYKLTKLSVKCYFNYSCVFSVVNATVTVNVTTHNGRVVNYTVCDDCNGYTDNIFSVQQDGRIPNGFPFNNWFLLTNDSTLVDGVSRLYQPLRLTCLWPVPGLKSSTGFVYFNATGSDVNCNGYQHNSVADVMRYNLNFSANSVDNLKSGVIVFKTLQYDVLFYCSNSSSGVLDTTIPFGPSSQPYYCFINSTINTTHVSTFVGVLPPTVREIVVARTGQFYINGFKYFDLGFIEAVNFNVTTASATDFWTVAFATFVDVLVNVSATKIQNLLYCDSPFEKLQCEHLQFGLQDGFYSANFLDDNVLPETYVALPIYYQHTDINFTATASFGGSCYVCKPHQVNISLNGNTSVCVRTSHFSIRYIYNRVKSGSPGDSSWHIYLKSGTCPFSFSKLNNFQKFKTICFSTVAVPGSCNFPLEATWHYTSYTIVGALYVTWSEGNSITGVPYPVSGIREFSNLVLNNCTKYNIYDYVGTGIIRSSNQSLAGGITYVSNSGNLLGFKNVSTGNIFIVTPCNQPDQVAVYQQSIIGAMTAVNESRYGLQNLLQLPNFYYVSNGGNNCTTAVMTYSNFGICADGSLIPVRPRNSSDNGISAIITANLSIPSNWTTSVQVEYLQITSTPIVVDCATYVCNGNPRCKNLLKQYTSACKTIEDALRLSAHLETNDVSSMLTFDSNAFSLANVTSFGDYNLSSVLPQRNIHSSRIAGRSALEDLLFSKVVTSGLGTVDVDYKSCTKGLSIADLACAQYYNGIMVLPGVADAERMAMYTGSLIGGMVLGGLTSAAAIPFSLALQARLNYVALQTDVLQENQKILAASFNKAINNIVASFSSVNDAITQTAEAIHTVTIALNKIQDVVNQQGSALNHLTSQLRHNFQAISNSIQAIYDRLDSIQADQQVDRLITGRLAALNAFVSQVLNKYTEVRSSRRLAQQKINECVKSQSNRYGFCGNGTHIFSIVNSAPDGLLFLHTVLLPTDYKNVKAWSGICVDGIYGYVLRQPNLVLYSDNGVFRVTSRVMFQPRLPVLSDFVQIYNCNVTFVNISRVELHTVIPDYVDVNKTLQEFAQNLPKYVKPNFDLTPFNLTYLNLSSELKQLEAKTASLFQTTVELQGLIDQINSTYVDLKLLNRFENYIKWPWWVWLIISVVFVVLLSLLVFCCLSTGCCGCCNCLTSSMRGCCDCGSTKLPYYEFEKVHVQ

SEQ ID NO: 54 - protein, 1171 aa, synthetic construct
MFVLLVAYALLHIAGCQTTNGTNTSHSVCNGCVGHSENVFAVESGGYIPSNFAFNNWFLLTNTSSVVDGVVRSFQPLLLNCLWSVSGSRFTTGFVYFNGTGRGDCKGFYSNASSDVIRYNINFEENLRRGTILFKTSYGAVVFYCTNNTLVSGDAHIPSGTVLGNFYCFVNTTIDNETTSAFVGALPKTVREFVISRTGHFYINGYRYFSLGNVEAVNFNVTNAATTVCTVALASYADVLVNVSQTAIANIIYCNSVINRLRCDQLSFDVPDGFYSTSPIQSVELPVSIVSLPVYHKHTFIVLYVNFELRRGPGRCYNCRPAVVNITLANFNETKGPLCVDTSHFTTQFVGNVKLDRWSASINTGNCPFSFGKVNNFVKFGSVCFSLKDIPGGCAMPIMANLVNHKSHNIGSLYVSWSDGDVITGVPKPVEGVSSFMNVTLNKCTKYNIYDVSGVGVIRISNDTFLNGITYTSTSGNLLGFKDVTNGTIYSITPCNPPDQLVVYQQAVVGAMLSENFTSYGFSNVVEMPKFFYASNGTYNCTDAVLTYSSFGVCADGSIIAVQPRNVSYDSVSAIVTANLSIPSNWTTSVQVEYLQITSTPIVVDCSTYVCNGNVRCVELLKQYTSACKTIEDALRNSAMLESADVSEMLTFDKKAFTLANVSSFGDYNLSSVIPSLPRSGSRVAGRSAIEDILFSKLVTSGLGTVDADYKKCTKGLSIADLACAQYYNGIMVLPGVADAERMAMYTGSLIGGIALGGLTSAASIPFSLAIQSRLNYVALQTDVLQENQKILAASFNKAMTNIVDAFTGVNDAITQTSQALQTVATALNKIQDVVNQQGNSLNHLTSQLRQNFQAISSSIQAIYDRLDIIQADQQVDRLITGRLAALNVFVSHTLTKYTEVRASRQLAQQKVNECVKSQSKRYGFCGNGTHIFSLVNAAPEGLVFLHTVLLPTQYKDVEAWSGLCVDGINGYVLRQPNLALYKEGNYYRITSRIMFEPRIPTIADFVQIENCNVTFVNISRSELQTIVPEYIDVNKTLQELSYKLPNYTVPDLVVEQYNQTILNLTSEISTLENKSAELNYTVQKLQTLIDNINSTLVDLKWLNRVETYIKWPWWVWLCISVVLIFVVSMLLLCCCSTGCCGFFSCFASSIRGCCESTKLPYYDVEKIHIQ

SEQ ID NO: 55 - protein, 1464 aa, synthetic construct
MIFIILTLLSVAKSEDAPHGVTLPQFNTSHNNERFELNFYNFLQTWDIPPNTETILGGYLPYCGAGVNCGWYNFSQSVGQNGKYAYINTQNLNIPNVHGVYFDVREHNNDGEWDDRDKVGLLIAIHGNSKYSLLMVLQDAVEANQPHVAVKICHWKPGNISSYHAFSVNLGDGGQCVFNQRFSLDTVLTTNDFYGFQWTDTYVDIYLGGTITKVWVDNDWSIVEASISYHWNRINYGYYMQFVNRTTYYAYNNTGGANYTQLQLSECHTDYCAGYAKNVFVPIDGKIPEDFSFSNWFLLSDKSTLVQGRVLSSQPVFVQCLRPVPSWSNNTAVVHFKNDAFCPNVTADVLRFNLNFSDTDVYTDSTNDEQLFFTFEDNTTASIACYSSANVTDFQPANNSVSHIPFGKTAHFCFANFSHSIVSRQFLGILPPTVREFAFGRDGSIFVNGYKYFSLPAIRSVNFSISSVEEYGFWTIAYTNYTDVMVDVNGTAITRLFYCDSPLNRIKCQQLKHELPDGFYSASMLVKKDLPKTFVTMPQFYHWMNVTLHVVLNDTEKKYDIILAKAPELAALADVHFEIAQANGSVTNVTSLCVQARQLALFYKYTSLQGLYTYSNLVELQNYDCPFSPQQFNNYLQFETLCFDVNPAVAGCKWSLVHDVQWRTQFATITVSYKHGSMITTHAKGHSWGFQDTSVLVKDECTDYNIYGFQGTGIIRNTTSRLVAGLYYTSISGDLLAFKNSTTGEIFTVVPCDLTAQVAVINDEIVGAITAVNQTDLFEFVNNTQARRSRSSTPNFVTSYTMPQFYYITKWNNDTSSNCTSAITYSSFAICNTGEIKYVNVTHVEIVDDSIGVIKPVSTGNISIPKNFTVAVQAEYIQIQVKPVVVDCATYVCNGNTHCLKLLTQYTSACQTIENALNLGARLESLMLNDMITVSDRGLELATVERFNATALGGEKLGGLYFDGLSSLLPPKIGKRSAVEDLLFNKVVTSGLGTVDDDYKKCSSGTDVADLVCAQYYNGIMVLPGVVDGNKMSMYTASLIGGMALGSITSAVAVPFAMQVQARLNYVALQTDVLQENQKILANAFNNAIGNITLALGKVSNAITTTSDGFNSMASALTKIQSVVNQQGEALSQLTSQLQKNFQAISSSIAEIYNRLEKVEADAQVDRLITGRLAALNAYVSQTLTQYAEVKASRQIALEKVNECVKSQSNRYGFCGNGTHLFSLVNSAPEGLLFFHTVLLPTEWEEVTAWSGICVNDTYAYVLKDFDHSIFSYNGTYMVTPRNMFQPRKPQMSDFVQITSCEVTFLNMTYTTFQEIVIDYIDINKTIADMLEQYNPNYTTPELNLLLDIFNQTKLNLTAEIDQLEQRADNLTTIAHELQQYIDNLNKTLVDLDWLNRIETYVKWPWYVWLLIGLVVVFCIPLLLFCCLSTGFCGCFGCVGSCCHSLCSRRQFETYEPIEKVHIH

SEQ ID NO: 56 - protein, 1386 aa, synthetic construct
MKSLNYFWLFLPVLSTFSLPQDVTRCSANTNFRRFFSKFNVQAPAVVVLGGYLPTGENHGVNSTWYCAGQHPTASGVHGVFLSHIRGGHGFEIGISQEPFDPSGYQLYLHKATNGNTNATARLRICQFPNIKRLGPTANNDVTTGRNCLFNKAIPAHMSEHSVVGVTWDNDRVTVFSDKIYHFYFKNDWSRVATKCYNSGGCAMQYVYEPTYYMLNVTSAGEDGISYQPCTANCIGYAANVFATEPNGHIPEGFSFNNWFLLSNDSTLLHGKVVSNQPLLVNCLLAIPKIYGLGQFFSFNQTMDGVCNGAAAQRAPEALRFNINDTSAILAEGSIVLHTALGTNLSFVCSNSSDTHLATFAIPLGATQVPYYCFLKVDTYNSTVYKFLAVLPPTVREIVITKYGDVYVNGFGYLHLGLLDAVTINFTGHGTDDDVSGFWTIASTNFVDALIEVQGTAIQRILYCDDPVSQLKCSQVAFDLDDGFYPISSRNLLSHEQPISFVTPPSFNDHSFVNITVSASFGGRSGANLIASDTTINGFSSFCVDTRQFTISLFYNVTNSYGYVSKSQDSNCPFTLQSVNDYLSFSKFCVSTSLLASACTIDLFGYPEFGSGVKFTSLYFQFTKGELITGTPKPLQGVTDVSFMTLDVCTKYTIYGFKGEGIITLTNSSFLAGVYYTSDSGQLLAFKNVTSGAVYSVTPCSFSEQAVYVDDDIVGVISSLSNSTFNNTRELPGFFYHSNDGSNCTEPVLVYSNIGVCKSGSIGYVPLQDGQVKIAPMVNGNISIPTNFSMSIRTEYLQLYNTPVSVDCVTYVCNGNSRCKQLLTQYTSACKTIESALQLSARLESVEVNSMLTISEEALQLATISSFNGDGYNFTNVLGVSVYDPASGRVVQKGSFIEDLLFNKVVTNGLGTVDEDYKRCSNGRSVADLVCAQYYSGVTVLPGVVDAEKLHMYSASLLGGMALGGLTTAAALPFSNAVQARLNYLALQTDVLQRNQQLLAESFNSAIGNITSAFESVKEAISQTSNGLNTVAHALTKVQEVVNSQGSALTQLTIQLQHNFQAISSSIDDIYSRLDILSADVQVDRLITGRLSALNAFVAQTLTKYTEVQASRKLAQQKVNECVKSQSQRYGFCGGDGEHIFSLVQAAPQGLLFLHTVLVPGDFVNVIAIDGLCVNGDIALTLREPGLVLFTHELQTYTATEYFVSSRRMFEPRKPTVSDFVQIESCVVTYVNLTSDQLPDVIPDYIDVNKTLDEIPASLPNRIGPSLPLDVFNATYLNLTGEIADLEQRSESLRNTTEELRSLIYNINNTLVDLEWLNRVETYIKWPWWVWLIIFIVLIFVVSLLVFCCISTGCCGCCGCCCACFSGCCRGPRLQPYEVFEKVHVQ

SEQ ID NO: 57 - protein, 1449 aa, synthetic construct
MKKLFVVLVVMPLIYGDNFPCSKLTNRTIGNHWNLIETFLLNYSSRLSPNSDVVLGDYFPTVQPWFNCIRNNSNDLYVTLENLKALYWDYATENITLNHKQRLNVVVNGYPYSITVTTTRNFNSAEGAIICICKGSPPTTTTESSLTCNWGSECRLNHKFPICPSNSEANCGNMLYGLQWFADAVVAYLHGASYRISFENQWSGTVTLGDMRATTLETAGTLVDLWWFNPVYDVSYYRVNNKNGTTVVSNCTDQCASYVANVFTTQPGGFIPSDFSFNNWFLLTNSSTLVSGKLVTKQPLLVNCLWPVPSFEEAASTLCFEGAGFDQCNGAVLNNTVDVIRFNLNFTTNVQSGKGATVFSLNTTGGVTLEISCYNDTVSDSSFSSYGEMPFGVTDGPRYCYVLYNGTALKYLGTLPPSVKEIAISKWGHFYINGYNFFSTFPIDCISFNLTTGDSDVFWTIAYTSYTEALVQVENTAITKVTYCNSYVNNIKCSQLTANLNNGFYPVSSSEVGLVNKSVVLLPSFYTHTIVNITIGLGMKRSGYGQPIASTLSNITLPMQDNNTDVYCIRSDQFSVYVHSTCKSSLWDNVFKRNCTDVLDATAVIKTGTCPFSFDKLNNYLTFNKFCLSLSPVGANCKFDVAARTRTNDQVVRSLYVIYEEGDNIVGVPSDNSGLHDLSVLHLDSCTDYNIYGRTGVGIIRQTNRTLLSGLYYTSLSGDLLGFKNVSDGVIYSVTPCDVSAQAAVIDGTIVGAITSINSELLGLTHWTTTPNFYYYSIYNYTNDRTRGTAIDSNDVDCEPVITYSNIGVCKNGALVFINVTHSDGDVQPISTGNVTIPTNFTISVQVEYIQVYTTPVSIDCSRYVCNGNPRCNKLLTQYVSACLTIEQALAMGARLENMEVDSMLFVSENALKLASVEAFNSSETLDPIYKEWPNIGGSWLEGLKYILPSDNSKRKYRSAIEDLLFAKVVTSGLGTVDEDYKRCTGGYDIADLVCAQYYNGIMVLPGVANADKMTMYTASLAGGITLGALGGGAVAIPFAVAVQARLNYVALQTDVLNKNQQILASAFNQAIGNITQSFGKVNDAIHQTSRGLATVAKALAKVQDVVNTQGQALSHLTVQLQNNFQAISSSISDIYNRLDELSADAQVDRLITGRLTALNAFVSQTLTRQAEVRASRQLAKDKVNECVRSQSQRFGFCGNGTHLFSLANAAPNGMIFFHTVLLPTAYETVTAWAGICALDGDRTFGLVVKDVQLTLFRNLDDKFYLTPRTMYQPRVATSSDFVQIEGCDVLFVNATVSDLPSIIPDYIDINQTVQDILENFRPNWTVPELTFDIFNATYLNLTGEIDDLEFRSEKLHNTTVELAILIDNINNTLVNLEWLNRIETYVKWPWYVWLLIGLVVIFCIPLLLFCCCSTGCCGCIGCLGSCCHSICSRRQFENYEPIEKVHVH

SEQ ID NO: 58 - protein, 1448 aa, synthetic construct
MKLLFVVLVVLPSIYGDNFPCSKFLNRTIGNHWNLIENFLLNYSIRLPPNSDVVLGDYFPTVQPWFNCIRNNNNSLYVTMENLKALYWDYATENITSDHRQRLHVVVKGKPYSITVTTTRNFDAAEGAIICICKGSPPTTTTGNLDCNWGSDCRLNHKFPICPSNSQANCGNMLYGLQWFTDEVVAYLHGAIYRISFENKWFGTVTLGDMRATTLQTAGALVDLWWFNPVYDVTYYRVNNKNGTTIVSNCTDQCASYVDNVFTTQPGGLIPSDFSFNNWFLLTNSSTVVSGKLVTRQPLVVNCLWPVPSFKEAASTFCFEGAGFDQCNGAVLNNTVDVIRFNLNFTADVQSGMGATVFSLNTTGGVILEISCYNDIVSESSFYSYGDIPFGITDGPRYCYVLYNGTTLKYLGTLPPSVKEIAISKWGHFYINGYNFFSTFPIDCISFNLTTGASGAFWTIAYTSYTEALVQVENTAIKKVTYCNSHINNIKCSQLTANLQNGFYPVASSEVGLVNKSVVLLPSFFAHTTVNITIDLGMKRSGYGQPIASPLSNITLPMQDNNTDVYCIRSNQFSIYVHSTCKSSLWDNVFNQDCTDVLEATAVIKTGTCPFSFDKLNNHLTFNKFCLSLSPVGANCKFDVAARTRTNEQVVRSLYVIYEEGDNIVGVPSDNSGLHDLSVLHLDSCTDYNIYGRTGVGVIRQTNSTLLSGLYYTSLSGDLLGFKNVSDGVIYSVTPCDVSAQAAVIDGAIVGAMTSINSELLGLTHWTTTPNFYYYSIYNYTNERTRGTAIDSNDVDCEPIITYSNIGVCKNGALVFINVTHSDGDVQPISTGNVTIPTNFTISVQVEYIQVYTTPVSIDCARYVCNGNPRCNKLLTQYVSACQTIEQALAMGARLENMEVDSMLFVSENALKLASVEAFNSTENLDPIYKEWPNIGGSWLGGLKDILPSHNSKRKYRSAIEDLLFDKVVTSGLGTVDEDYKRCTGGYDIADLVCAQYYNGIMVLPGVANDDKMTMYTASLAGGITLGALGGGAVAIPFAVAVQARLNYVALQTDVLNKNQQILANAFNQAIGNITQAFGKVNDAIHQTSKGLATVAKVLAKVQDVVNTQGQALSHLTVQLQNNFQAISSSISDIYNRLDELSADAQVDRLITGRLTALNAFVSQTLTRQAEVRASRQLAKDKVNECVRSQSQRFGFCGNGTHLFSLANAAPNGMIFFHTVLLPTAYQTVTACSGICASDGDRTFGLVVKDVQLTLFRNLDDKFYLTPRTMYQPRAATSSDFVQIEGCDVLFVNATEIDLPSIIPDYIDINQTVQDILENYRPNWTVPELTLDIFNATYLNLTGEIDDLEFRSEKLHNTTVELAILIDNINNTLVNLEWLNRIETYVKWPWYVWLLIGLVVVFCIPLLLFCCCSTGCCGCIGCLGSCCHSICSRRQFENYEPIEKVHVH

SEQ ID NO: 59 - protein, 1179 aa, synthetic construct
MQRIILISTILYCARALTLADKMLDLLTFPGAHHYFRGDLQTLHSRISAESYSVNPYDQYNYQTDSDYYINKSVHLIAPLTNLTLPISGLHRSMQPLRVGCIFGASNKIDQGFTISGMTYPLAYCVPPFYQVTNVTYDAMRLLFAFADLNSTGDFLRINTKTMGMLNVSCSASPTPLGHQDADRTFYGYNKQLYCYLDTPAGMQYMGPLPANLTEITLFRTGQIYTNGFHLGTIPSELTYVYLDKLAFQNKTVCMMANLTDTLITLNHTVIQQVTYCEKDAVQALACQQSTHQLQDGFYSDPAPAVNNLPKTLVTLPKIAESSTLQINVSATYSYGSASGSIKLSYNGSSNNSHCVQTPYFKLEQNLVCSGGCSVRIETLTCPFDLNAVSNGMSFQQFCVSTVSGQCSMQAIVNTGQPWGYVTSTLYVTYVEGQSFTGTSSDQIEDLTVLHLDQCTSYTIYGVSGTGVITLSDLQLPHGITFRAANGELSAFKNTTTGDVYTIQPCSLPAQLAIIDSTIVGAITSTNESYGFSNTIVTPTFYYSTNATSNCTAPKISYGELGVCADGSIGAVSQLQDSKPSIVPLYTGEIEIPASFKLSVQTEYLQVQTEQVVIDCPKYVCNGNPRCLQLLAQYTSACSNIESALHSSAQLDSREITMMFQTSSQSVELANITNFQGDYNFSMILPTLPGKDRSAIEDLLFDKVVTNGLGTVDQDYKSCSKGIAVADLVCAQYYNGIMVLPGVVDAEKMAMYTGSLTGAMVFGGLTAAAAIPFSTAVQARLNYVALQTNVLQENQKILAESFNQAVGNISLALSNVNTAIQQTSEALLTVSNAINKIQTVVNQQGEALAHLTAQLSQNFQAISTSIQDIYNRLDQIQADQQVDRLITGRLAALNAYVTQLLNKLSQVRQSRILAEQKINECVKSQSSRYGFCGNGTHLFSLTQAAPNGIFFMHAVLVPQTFQPVVAYAGICVDGYGYSLQPQLVLYNLNDSYRITPRNMFEPRTPTQSVFIPLTTCSVDFVNVTANNVSIIIPDYVDVNKTVSDIINGLPNYSYPELSLDRFNHTILNLSQEIEDLQIRSQNLSATAELLQQYIDNLNNTLVDLEWLNRVETYLKWPWYIWLLIFLAIAAFATILVTIFLCTGCCGGCFGCCGGCFGLFSKKRRLSSEPTPVSFKLKEW

SEQ ID NO: 60 - protein, 1254 aa, synthetic construct
MQCVVLVLTLISIVTSRPNSFADRVFDALTFPHASNYLHVGDKTPSRPQLLQARNQGNYSAICPENGYITSTSYDLSKIYYLTDGDYPIDGVYKSLQPLKVTCVPEWHGNSNFNDTTGWKHYFDGRIKQNPNTIWCPCSQSGPGGAQHNAAGNSTEYIRFHSNITTSVSNLLRLYAVDNQYYYFGCTPTPTPLTFNLTSENITLFTAEEQVHYCYANINGTVSYIGVLPPKITELTVGRYGDIFVNGFLYFKIPNVIEYVQLSHTIPHNKQFYTVFYANMTQVLLNISMASINRLLYCDKDSYSSIACAVNQFEPANGFYSTSAIEKISRKFTFVTMPTVQNHSYYSINLTIGGCGHGEYPHLSNKTGCYRTDASNINAKQITFVINTYTHDNWIQWAHKPGNCPWALNKINNYNTAGTLQVVPENQATCCTDNQASWLYLASWTSVNVKVCFNYQPGTTISIQPQQTGVATDISVIYENECVDYNIYGKTGTGIIQSTNVTLLAGRTYTSASGQLLAFKYLSNQTIYSVTPCDFSNQVAVYNKSVIAAILPQNKTIFGLTNIQETPNFYIANNAHQQQRFAMYMEPLNSQQPDCTPVLTYAQIGICADGQFVQVQPEKSQPMSTTPIVAVNITIPKTFNISVQTEYIQISTDNIVIDCQRYVCNGNPRCLMLLSQYQSACSTIEQALHQKARLESLELSTMLAYSPNTLQLANVSNFQSNNMGFNLTNLLPQNNSPQKRSVIEDLLFSKVVTNGLGTVDVDYKKCTKGLSIADLPCAQYYNGIMVLPGVADSGLLAAYTASLTGGMVFGGLTSAAAIPFATAVQARLNYVALQTDVLQRNQQILANAFNQAMGNITLAFKDVKEAIATTADAIRVVAGALDKIQQVVNSQGQALSKLTGELQRNFQAISASIEDIYNRLNDIEADAQVDRLITGRLAALNAFLTQTLTQANEVKAARELALQKINECVKDQSKRYGFCGNGYHLFSIANAAPKGFIFFHTVLQPETTIEIQAIAGFCVSDRQTYNYYSSNMEGQAYIARDTTQTIFLHENGTYMITPRKQYQPRTLAQADVVKISTCDVTYVNLTSIEFEQLIPEYVDINSTVEGILNSTLPGKIPDLNIGHYNNTILNLTTEINDLQSKAENLSMIAYQLEEYIKNINNTLVDLEWLNRVETYLKWPWYVWLAIALAFTGFVTILITIFLCTGCCGGCFGCCGGCFGLFSKKIDPMRQYMNRYETPTSKSDDAIPIIYKKNW

SEQ ID NO: 61 - protein, 1160 aa, synthetic construct
MQRALLIMTLLSLARAKFADDLLDLLTFPGAHRFLHKPTRNSSNLYLRANNNFDVGVLPGYPTKNVNLFSPLTNSTLPINGLHRSYQPLMLNCLTKITNHTLSMYLLPSEVQTYSCGGAMVKYQTHDAVRIILDLIATDHISVEVVGQRGENYVFVCSEQFNYTTALHNATFFSLNSQLYCFTNNTYLGILPPDLTDFTVYRTGQFYANGYLLGTLPITVNYVRLYRGQLAANSAHFALANLTDTLITLTNTTISQITYCDKSVVDSIACQRSSHEVEDGFYSDPKSAVRARQRTIVTLPKLPELEVVQLNISAHMDFGEARLDSVTINGNTSYCVTKPYFRLETNFMCTGCTMNLRTDTCSFDLSAVNNGMSFSQFCLSTESGACEMKIIVTYVWNYLLRQRLYVTAVEGQTHTGTTSVHATDTSSVITGVCTDYTIYGVSGTGIIKPSDLLLHNGIAFTSPTGELYAFKNITTGKTLQVLPCETPSQLIVINNTVVGAITSSNSTENNRFTTTIVTPTFFYSTNATTFNCTKPVLSYGPISVCSDGAIVGTSTLQNTRPSIVSLYDGEIEIPSAFSLSVQTEYLQVQAEQVIVDCPQYVCNGNSRCLQLLAQYTSACSNIEAALHSSAQLDSREIINMFKTSTQSLQLANITNFKGDYNFSSIITPRVGGRSAIEDLLFNKVVTSGLGTVDQDYKSCSRDMAIADLVCSQYYNGIMVLPGVVDAEKMAMYTGSLTGAMVFGGLTAAAAIPFATAVQARLNYVALQTNVLQENQKILAESFNQAVGNISLALSSVNDAIQQTSEALNTVAIAIKKIQTVVNQQGEALSHLTAQLSNNFQAISTSIQDIYNRLEEVEANQQVDRLITGRLAALNAYVTQLLNQMSQIRQSRLLAQQKINECVKSQSPRYGFCGNGTHIFSLTQTAPNGIFFMHAVLVPNKFTRVNASAGICVDNTRGYSLQPQLILYQFNNSWRVTPRNMYEPRLPRQADFIQLTDCSVTFYNTTAANLPNIIPDVIDVNQTVSDIIDNLPTATPPQWDVGIYNNTILNLTVEINDLQERSKNLSQIADRLQNYIDNLNNTLVDLEWLNKVETYLKWPWYVWLAIALALIAFVTILITIFLCTGCCGGCFGCCGGCFGLFSKKKRYTDDQPTPSFKFKEW

SEQ ID NO: 62 - protein, 1159 aa, synthetic construct
MQRALLIMTLLCLVRAKFADDLLDLLTFPGAHRFLHKLTSNSSSFYSRANNFDVGVLPGYPTENVNLFSPLTNSTLPINGLHRSYQPLMLNCLTKITNHTLSMYLLPSEIQTYSCGGAMVKYQTHDAVRIILDLTVTDHISVEVVGQRGENYVFVCSEQFNYTTALHNSTFFSLNSELYCFTNNTYLGILPPDLTDFTVYRTGQFYANGYLLGTLPITVNYVRLYRGHLSANSAHFALANLTDTLITLTNTTISQITYCDKSVVDSIACQRSSHEVEDGFYSDPKSAVRARQRTIVTLPKLPELEVVQLNISAHMDFGEARLDSVTINGNTSYCATKPYFRLETNFMCTGCTMNLRTDTCSFDLSAVNNGMSFSQFCLSTESGACEMKIIVTYVWNYLLRQRLYVTAVEGQTHTGTTSVHATDTSSVITDVCTDYTIYGVSGTGIIKPSDLLLHNGIAFTSPTGELYAFKNITTGKTLQVLPCETPSQLIVINNTVVGAITSSNSTENNRFTTTIVTPTFFYSTNATTFNCTKPVLSYGPISVCSDGAIAGTSTLQNTRPSIVSLYDGEVEIPSAFSLSVQTEYLQVQAEQVIVDCPQYVCNGNSRCLQLLAQYTSACSNIEAALHSSAQLDSREIINMFQTSTQSLQLANITNFKGDYNFSSILTTRLGGRSAIEDLLFNKVVTSGLGTVDQDYKACSRDMAIADLVCSQYYNGIMVLPGVVDAEKMAMYTGSLTGAMVFGGLTAAAAIPFATAVQARLNYVALQTNVLQENQKILAESFNQAVGNISLALSSVNDAIQQTSEALNTVAIAIKKIQTVVNQQGEALSHLTAQLSNNFQAISTSIQDIYNRLEEVEANQQVDRLITGRLAALNAYVTQLLNQMSQIRQSRLLAQQKINECVKSQSSRYGFCGNGTHIFSLTQTAPNGIFFMHAVLVPNKFTRVNASAGICVDNIKGYSLQPQLILYQFNNSWRVTPRNMYEPRLPRQADFIQLTDCSVTFYNTTAANLPNIIPDIIDVNQTVSDIIDNLPTATPPQWDVGIYNNTILNLTVEINDLQERSKNLSQIADRLQNYIDNLNNTLVDLEWLNRVEIYLKWPWYIWLAIALALIAFVTILITIFLCTGCCGGCFGCCGGCFGLFSKKKRYTDDQPTPSFKFKEW

SEQ ID NO: 63 - protein, 32 aa, synthetic construct
PIDISIELNKAKSDLEESKEWIRRSNQKLDSI

SEQ ID NO: 64 - protein, 32 aa, synthetic construct
PVDISLNLAAATDFLQDSRAELEKARKILSEV

SEQ ID NO: 65 - protein, 32 aa, synthetic construct
PVDISLNLASATNFLEESKTELMKARAIISAV

SEQ ID NO: 66 - protein, 32 aa, synthetic construct
PLDLSNQINSINKSLKSAEDWIADSNFFANQA

SEQ ID NO: 67 - protein, 32 aa, synthetic construct
PLDLSTDLNQYNQLLKSAEDHIQRSTDYLNSI

SEQ ID NO: 68 - protein, 32 aa, synthetic construct
PLDISQNLAAVNKSLSDALQHLAQSDTYLSAI

SEQ ID NO: 69 - protein, 32 aa, synthetic construct
PIDISTELSKVNASLQNAVKYIKESNQQLQSV

SEQ ID NO: 70 - protein, 32 aa, synthetic construct
RLDVGTNLGNAIAKLEDAKELLESSDQILRSM

SEQ ID NO: 71 - protein, 32 aa, synthetic construct
KVDISSQISSMNQSLQQSKDYIKEAQRLLDTV

SEQ ID NO: 72 - protein, 32 aa, synthetic construct
KVDISSQISSMNQSLQQSKDYIKEAQKILDTV

SEQ ID NO: 73 - protein, 32 aa, synthetic construct
NLDISTELGNVNNSISNALNKLEESNRKLDKV

SEQ ID NO: 74 - protein, 32 aa, synthetic construct
NLDISTELGNVNNSISNALDRLAESSSKLERV

SEQ ID NO: 75 - protein, 32 aa, synthetic construct
RLDVGTNLGNALKKLDDAKVLIDSSNQILETV

SEQ ID NO: 76 - protein, 32 aa, synthetic construct
KLDVGTDLAIASAKLNNSKALLEQSDKILYSM

SEQ ID NO: 77 - protein, 32 aa, synthetic construct
PVDISLQIVAATNFLNSSRKDLMKAKEVLNQV

SEQ ID NO: 78 - protein, 32 aa, synthetic construct
KIDIGNQLAGINQTLQEAEDYIEKSEEFLKGV

SEQ ID NO: 79 - protein, 32 aa, synthetic construct
PIDISGALGKIEQDLQSSKEHLANSDTILASI

SEQ ID NO: 80 - protein, 32 aa, synthetic construct
KLDVGTNLGNALTKLNNAKELLDSSNQILENV

SEQ ID NO: 81 - protein, 32 aa, synthetic construct
DLDLSAELTKVQSDLKEAQDRLDESNSILQGI

SEQ ID NO: 82 - protein, 32 aa, synthetic construct
KIDIGNQLAGINQTLQNAEDYIEKSEEFLKGI

SEQ ID NO: 83 - protein, 52 aa, synthetic construct
YLNLSSELKQLEAKTASLFQTTVELQGLIDQINSTYVDLKLLNRFENYIKWP

SEQ ID NO: 84 - protein, 52 aa, synthetic construct
ILNLTSEISTLENKSAELNYTVQKLQTLIDNINSTLVDLKWLNRVETYIKWP

SEQ ID NO: 85 - protein, 52 aa, synthetic construct
KLNLTAEIDQLEQRADNLTTIAHELQQYIDNLNKTLVDLDWLNRIETYVKWP

SEQ ID NO: 86 - protein, 52 aa, synthetic construct
YLNLTGEIADLEQRSESLRNTTEELRSLIYNINNTLVDLEWLNRVETYIKWP

SEQ ID NO: 87 - protein, 52 aa, synthetic construct
YLNLTGEIDDLEFRSEKLHNTTVELAILIDNINNTLVNLEWLNRIETYVKWP

SEQ ID NO: 88 - protein, 52 aa, synthetic construct; identical to SEQ ID NO: 87

SEQ ID NO: 89 - protein, 50 aa, synthetic construct
APDLSLDYINVTFLDLQVEMNRLQEAIKVLNHSYINLKDIGTYEYYVKWP

SEQ ID NO: 90 - protein, 52 aa, synthetic construct
APNLTLNLHTINATFLDLYYEMNLIQESIKSLNNSYINLKDIGTYEMYVKWP

SEQ ID NO: 91 - protein, 51 aa, synthetic construct
DVDLGDISGINASVVNIQKEIDRLNEVAKNLNESLIDLQELGKYEQYIKWP

SEQ ID NO: 92 - protein, 51 aa, synthetic construct; identical to SEQ ID NO: 91

SEQ ID NO: 93 - protein, 51 aa, synthetic construct
IPNFGSLTQINTTLLDLTYEMLSLQQVVKALNESYIDLKELGNYTYYNKWP

SEQ ID NO: 94 - protein, 51 aa, synthetic construct
GPNFAEISKINTTLLDLSDEMAILQEVVKQLNDSYIDLKELGNYTYYNKWP

SEQ ID NO: 95 - protein, 51 aa, synthetic construct
FNNTFNPNDFNFSTVDVTAQIKSLHDVVNQLNQSFIDLKKLNVYEKTIKWP

SEQ ID NO: 96 - protein, 38 aa, synthetic construct
ILNISGEIDHIQGVIQGLNDSLINLEELSILKTYIKWP

SEQ ID NO: 97 - protein, 45 aa, synthetic construct
YLNVSDQIDIINGAMENIRNQIQNLNSSYIDLEWLNKYERYSKWP

SEQ ID NO: 98 - protein, 52 aa, synthetic construct
ILNLSQEIEDLQIRSQNLSATAELLQQYIDNLNNTLVDLEWLNRVETYLKWP

SEQ ID NO: 99 - protein, 52 aa, synthetic construct
ILNLTTEINDLQSKAENLSMIAYQLEEYIKNINNTLVDLEWLNRVETYLKWP

SEQ ID NO: 100 - protein, 52 aa, synthetic construct
ILNLTVEINDLQERSKNLSQIADRLQNYIDNLNNTLVDLEWLNKVETYLKWP

SEQ ID NO: 101 - protein, 52 aa, synthetic construct
ILNLTVEINDLQERSKNLSQIADRLQNYIDNLNNTLVDLEWLNRVEIYLKWP

(19) United States
(12) Patent Application Publication
(10) Pub. No.: US 2026/0085096 A1
(43) Pub. Date: Mar. 26, 2026

(54) STABILIZED TRIMERIC CLASS I FUSION PROTEINS

(71) Applicant: JANSSEN VACCINES & PREVENTION B.V., Leiden (NL)

(72) Inventors: Johannes Petrus Maria LANGEDIJK, Amsterdam (NL); Mark Johannes Gerardus BAKKERS, Haarsteeg (NL); Annemart KOORNNEEF, Roelofarendsveen (NL); Jaroslaw JURASZEK, Duivendrecht (NL)

(21) Appl. No.: 19/113,685

(22) PCT Filed: Sep. 15, 2023
(86) PCT No.: PCT/EP2023/075392

§ 371 Date: Mar. 20, 2025

(30) Foreign Application Priority Data
Sep. 23, 2022 (EP) 22197415.7

(51) Int. Cl. C07K 14/005; C12N 15/86

(52) U.S. Cl. CPC C07K 14/005; C12N 15/86; C12N 2710/10343; C12N 2760/18222; C12N 2760/18622; C12N 2770/20022

(57) ABSTRACT

The present invention relates to trimeric class I fusion proteins, in particular to class I fusion proteins, comprising at least one stabilizing mutation in the HR2 domain, and to fragments thereof.

The present invention relates to the field of medicine. The invention, in particular, relates to recombinant trimeric class I fusion proteins from paramyxoviridae and coronaviridae, to nucleic acid molecules encoding the class I fusion proteins, such as paramyxovirus F and coronavirus S proteins, and to uses thereof, e.g. in vaccines.

### BACKGROUND OF THE INVENTION

Many circulating human pathogenic viruses are enveloped viruses that infect host cells by inducing fusion of the virion envelope with a host cell membrane. The viral fusion protein is key in mediating viral entry by functioning as a dynamic fusion machine that drives membrane fusion by irreversible protein refolding from a metastable pre-fusion conformation to a stable post-fusion conformation.

Since viral fusion proteins can mediate both virus-cell fusion leading to infection and pathological cell-cell fusion, they are increasingly viewed as targets for antiviral intervention. When viral fusion proteins are used as a vaccine component their fusogenic function is not relevant. In fact, only the mimicry of the component is important to induce cross-reactive antibodies that can bind the virus. Structure-based stabilization of viral fusion proteins in the pre-fusion conformation has been shown to induce superior neutralization and protection in animal models and clinical trials (Krarup et al. (2015) Nat Commun, 6:8143; De Taeye et al. (2015), Cell, 163:1702-1715; Mclellan et al. (2013), Science, 342:592-598; Stewart-Jones et al. (2018). Proc Natl Acad Sci USA, 48:12265-12270; Crank et al., (2019), Science, 365:505-509; Sadoff et al. (2021), J Infect Dis, doi: 10.1093/infdis/jiab003; Sadoff et al. (2021), N Engl J Med, 384; 2187-2201). Therefore, for development of robust efficacious vaccine components it is desirable that the meta-stable fusion proteins are maintained in their pre-fusion conformation.

A soluble, subunit-based vaccine requires truncation of the viral fusion protein by deletion of the transmembrane (TM) and the cytoplasmic region. The remaining ectodomain of the fusion protein is considerably more labile due to removal of the membrane anchor and will either not form as a trimeric protein or even more readily refold into the post-fusion end-state. Therefore, soluble viral fusion proteins are commonly equipped with a C-terminal heterologous trimerization domain (e.g. foldon or GCN4) to restore trimer formation (Welch et al. (2012), Proc Natl Acad Sci USA, 109:16672-16677; Mclellan et al. (2013), Science, 342:592-598; Walls et al. (2016), Nat Struct Mol Biol, 23:899-905). However, addition of a non-native trimerization domain may induce non-relevant antibodies to this domain that will not cross-react with the virus and may hamper immunogenicity when the domain is used in other vaccines, which will increase its immunodominance.

Therefore, a need remains for e.g. vaccine components in a pre-fusion conformation that do not require a heterologous trimerization domain. The present invention aims at providing means for obtaining such stable pre-fusion proteins for use in vaccinating against pathogenic viruses.

### SUMMARY OF THE INVENTION

The present invention provides trimeric (soluble) class I viral fusion proteins that are stabilized in the pre-fusion conformation.

In a first aspect, the invention provides soluble class I fusion proteins, or fragments thereof, comprising at least one stabilizing mutation in the HR2 domain, wherein the protein is not a PIV-3 protein, without a heterologous trimerization domain. The present invention thus provides trimeric class I fusion proteins that are stabilized in the pre-fusion conformation without the need for heterologous trimerization domains (e.g. GCN4, foldon), that have improved percentage of trimer formation and/or improved trimer yields and/or improved (thermal) stability as compared to previously described class I fusion proteins.

In a second aspect, the invention provides full-length stabilized trimeric class I fusion proteins, comprising at least one stabilizing mutation in the HR2 domain. Because also the full length (membrane-bound) class I fusion proteins are metastable, the stabilization of the trimeric pre-fusion conformation is also desirable for full-length class I fusion proteins, i.e. including the TM and cytoplasmic region, e.g. for any DNA, RNA, live attenuated or vector-based vaccine approach.

The invention also provides nucleic acid molecules encoding the class I fusion proteins, and fragments thereof, such as DNA, RNA, mRNA, as well as vectors comprising such nucleic acid molecules.

The invention moreover provides compositions, preferably pharmaceutical compositions, such as e.g. vaccines, comprising a class I fusion protein, or a fragment thereof, a nucleic acid molecule and/or a vector, as described herein.

The invention also provides compositions for use in inducing an immune response against a class I fusion protein, and in particular to the use thereof as a vaccine.

The invention also relates to methods for inducing an immune response against a class I fusion protein in a subject, comprising administering to the subject an effective amount of a class I fusion protein or a fragment thereof, a nucleic acid molecule encoding said class I fusion protein, and/or a vector comprising said nucleic acid molecule, as described herein.

The invention also relates to the use of the class I fusion proteins or fragments thereof, as described herein, for isolating monoclonal antibodies against said class I fusion protein from infected humans.

Another general aspect relates to a host cell comprising the isolated nucleic acid molecule or vector encoding the recombinant class I fusion protein of the invention. Such host cells can be used for recombinant protein production, recombinant protein expression, or the production of protein particles or viral particles.

Another general aspect relates to methods of producing a recombinant class I fusion protein, comprising growing a host cell comprising an isolated nucleic acid molecule or vector encoding the recombinant class I fusion protein of the invention under conditions suitable for production of the recombinant class I fusion protein.

### BRIEF DESCRIPTION OF THE FIGURES

The foregoing summary, as well as the following detailed description of the invention, will be better understood when read in conjunction with the appended figures. It should be understood that the invention is not limited to the precise embodiments shown in the examples.

FIG. 1: HR2 optimization of Nipah F in the absence of a heterologous trimerization domain.

(A) Schematic overview of soluble Nipah F designs.

(B) Nipah F trimer detection in cell culture supernatant using analytical size exclusion chromatography (SEC). The peak at approximately 4.5 minutes retention time corresponds to the Nipah F trimer.

FIG. 2: HR2 optimization of Nipah F in the absence of a heterologous trimerization domain, in combination with additional backbone mutations.

(A) Schematic overview of soluble Nipah F designs.

(B) Nipah F trimer detection in cell culture supernatant using analytical SEC. The peak at approximately 4.4- and 4.6-minutes retention time corresponds to the Nipah F trimer with (T+GCN4) and without (T−GCN4) trimerization domain, respectively.

(C) Expression of Nipah F in supernatant using quantitative Octet. The initial binding rate and average of two replicates per design is reported after mock subtraction, with error bar indicating the standard deviation.

(D) Analytical SEC-MALS analysis of purified Nipah F trimers.

(E) Melting temperature (Tm50) of purified Nipah F trimers as determined by Differential Scanning Fluorimetry (DSF). N=3 replicate measurements, and individual and average values are reported as grey and black solid lines, respectively.

FIG. 3. HR2 optimization of full-length membrane-bound Nipah F prevents cell-cell fusion.

A) Schematic overview of full-length membrane-bound Nipah F designs.

(B) Results of the cell-cell fusion assay of full-length membrane-bound Nipah F designs. Relative cell-cell fusion is scored as −/ +/ ++/+++, to signify increasing syncytia formation.

FIG. 4. HR2 optimization of HPIV3 F in the absence of a heterologous trimerization domain.

(A) Schematic overview of soluble HPIV3 F designs.

(B) HPIV3 F trimer detection in cell culture supernatant using analytical SEC. The peak at approximately 4.7 minutes retention time corresponds to the HPIV3 F trimer.

(C) Expression of HPIV3 F in cell culture supernatant using quantitative Octet. Initial binding rate is plotted after subtraction of mock signal.

FIG. 5. HR2 and head domain optimization of HPIV1 F in the absence of a heterologous trimerization domain.

(A) Soluble HPIV1 F trimer detection in cell culture supernatant using analytical SEC. The peak at approximately 4.5 minutes retention time corresponds to the HPIV1 F trimer.

(B) Analytical SEC-MALS of purified HPIV1 F trimer without a heterologous trimerization domain.

(C) Melting temperature of purified HPIV1 F trimer as determined by DSF. N=3 replicate measurements, and individual and average values are reported as grey and black solid lines, respectively.

(D) HPIV1 F trimer detection in cell culture supernatant using analytical SEC. The peak between 4.4- and 4.7-minutes retention time corresponds to the HPIV1 F trimer.

(E) Melting temperature of HPIV1 F trimers in cell culture supernatant as determined by DSF. N=3 replicate measurements, and the average per design is reported with error bar indicating the standard deviation.

FIG. 6. HR2 optimization of NL63 S in the absence of a heterologous trimerization domain.

(A) Schematic overview of soluble NL63 S designs.

(B) NL63 S trimer and monomer detection in cell culture supernatant using analytical SEC. The peak between 5- and 5.5-minutes retention time corresponds to the NL63 S trimer and the peak between 5.5- and 6-minutes retention time corresponds to the NL63 S monomer. OD214 values were displayed after subtraction of OD214 signal of mock-transfected cells.

(C) NL63 S trimer and monomer detection in cell culture supernatant using analytical SEC. OD214 values were displayed after subtraction of OD214 signal of mock-transfected cells.

(D) NL63 S trimer and monomer detection in cell culture supernatant using analytical SEC at the day of harvest (t=0) or after 9 days storage at 4° C. (t=9). OD214 values were displayed after subtraction of OD214 signal of mock-transfected cells.

(E) Analytical SEC-MALS analysis of purified NL63 S trimer without a heterologous trimerization domain.

(F) Melting temperatures Tm1 and Tm2 of purified NL63 S trimer as determined by DSF. N=3 replicate measurements, and individual and average values are reported as grey and black solid lines, respectively.

FIG. 7: Heptad repeat similarity in different class I fusion proteins incorporating a HR2 coiled-coil.

(A) Cartoon representation of paramyxovirus and coronavirus class I fusion proteins with a parallel HR2 coiled-coil. The region that can be stabilized to generate trimers that do not need heterologous trimerization domains is encircled.

(B) Alignment of HR2 of paramyxovirus F proteins. The heptad repeat is shown as bold text and the register is indicated. Optimal residues at positions a and d are highlighted in black and potential sub-optimal residues are highlighted in gray. The position of amino acids 470 and 477, according to numbering in SEQ ID NO: 1 is indicated above the alignment.

(C) Alignment of HR2 of coronavirus S proteins. The heptad repeat is shown as bold text and the register is indicated. Optimal residues at positions a and d are highlighted in black and potential sub-optimal residues are highlighted in gray. The position of amino acids 1259 and 1266, according to numbering in SEQ ID NO:53 is indicated above the alignment.

### DETAILED DESCRIPTION OF THE INVENTION

Enveloped viruses initiate infection by fusing their membrane with the cell membrane and thereby depositing their genome into the cytosol. This membrane merger is catalyzed by specialized viral proteins referred to as fusion proteins. A salient feature of viral fusion proteins is a highly conserved, functionally important stretch of hydrophobic residues referred to as the fusion peptide or the fusion domain. When triggered by receptor binding and/or by low pH, viral fusion proteins insert their fusion peptides into the target membrane, promoting the fusion of the viral and the target membranes by undergoing complex conformational changes.

Based on the structure of extracellular domains, viral fusion proteins are categorized into three classes. Fusion proteins of retroviruses, filoviruses, coronaviruses, ortho- and paramyxoviruses displaying a prevalent α-helical motif belong to the class I proteins. In addition, heptad repeat (HR) regions, which are found in fusion proteins of many different viruses, also form an important characteristic of class I viral fusion proteins There are usually two HR regions; one N-terminal HR region (HR1) adjacent to the fusion peptide and a C-terminal HR region (HR2) close to the transmembrane anchor. Structural studies of viral fusion proteins have revealed that the HR regions in the post-fusion conformation form a six-helix bundle (6HB) structure which is implicated in viral entry. The structure consists of a homo-trimeric coiled-coil of HR1 domains, in the exposed hydrophobic grooves of which the HR2 regions are packed in an antiparallel manner. This conformation brings the N-terminal fusion peptide into close proximity of the transmembrane anchor. Because the fusion peptide inserts into the cell membrane during the fusion event, such a conformation facilitates a close apposition of the cellular and viral membrane. It has thus been suggested that the actual six-helix bundle formation is directly coupled to the merging of the membranes.

As described above, pre-fusion class I viral fusion proteins have a metastable nature which promotes refolding into the post-fusion state and formation of the 6HB. For the development of efficacious vaccine components it is desirable that the meta-stable fusion proteins are maintained in their pre-fusion conformation. According to the present invention, it has been found that the pre-fusion conformation of class I fusion proteins can be stabilized by stabilizing the HR2 region (also known as HRB). Heptad repeat (HR) domains or motifs are the basis for coiled-coils, which are versatile structural elements that are widely used in many protein families. The heptad repeat register driving the helical structure assembly is usually denoted abcdefg, and can be identified by bioinformatic analyses, with hydrophobic amino acids at positions a and d of the repeats (Lupas and Gruber (2005), Adv Protein Chem, 70:37-78). Generally, natural coiled-coil sequences tend to use the aliphatic hydrophobic amino acid residues (A, I, L, M, V) at these positions, rather than amino acids with aromatic hydrophobic side chains (F, W, Y). The remaining b, c, e, f, and g sites are more permissive than the a and d sites, though polar and helix-favoring residues (A, E, K, Q) tend to be favored for these positions (Woolfson (2005), Adv Protein Chem, 70:79-112). The main properties that provide stability to a coiled-coil are helical propensity, hydrophobicity of the core, tightness of the core packing, shielding of the core from solvent, and favorable polar and ionic interactions (Lupas and Gruber (2005), Adv Protein Chem, 70:37-78). According to the invention, HR2 stabilization involves the mutation (substitution) of sub-optimal amino acids (e.g. hydrophilic amino acids) at positions a and/or d of the heptad repeat into hydrophobic amino acid residues, preferably with branched-chain amino acids (BCAAs) V, I, or L, or with M, thereby stabilizing the hydrophobic core of the coiled-coil structure.

It is noted that the terms substitution and mutation are used interchangeably throughout this application.

The present invention thus provides class I fusion proteins and fragments thereof, comprising at least one stabilizing mutation in the HR2 domain.

In certain embodiments, the at least one stabilizing mutation is a mutation of an amino acid residue selected from the group consisting of S, T, A and Y at positions a and/or d of the heptad repeat motif into a hydrophobic amino acid residue.

The hydrophobic amino acid residue can be any hydrophobic amino acid residue, preferably any branched-chain amino acid (BCAA), such as V, I, or L. In certain embodiments, the hydrophobic amino acid residue is an amino acid selected from the group consisting of V, I, M and L. Preferably, the hydrophobic amino acid is V.

In certain embodiments, the at least one stabilizing mutation in the HR2 domain improves trimer expression and/or stabilizes the pre-fusion conformation.

In certain embodiments, the class I fusion protein is a class I fusion protein that has a HR2 stem region that consists of parallel alpha helices that form a coiled-coil structure (parallel coiled-coils) (Woolfson et al. (2012) Curr Opin Struct Biol, 22:432-441). According to the invention, it has been found that this stabilization method is applicable to all class I fusion proteins that have HR2 stem regions consisting of a parallel coiled-coil, including, but not limited to, paramyxovirus F and coronavirus S proteins. Physical demonstration or computer prediction of such coiled-coil structure may be used to help define if a fusion protein has a HR2 stem region that consists of parallel coiled-coils (Carr et al., Cell 73, 823-832 (1993)).

In certain preferred embodiments, the class I fusion protein is a paramyxovirus fusion (F) or a coronavirus spike(S) protein.

In certain embodiments, the paramyxovirus F protein is an F protein from a virus selected from the group consisting of Nipah virus, Sendai virus, PIV-1, PIV-2, PIV-4, PIV-5, Mumps virus, Measles virus, Hendra virus, Newcastle disease virus, Avian orthoavulavirus, Canine distemper virus, Feline morbillivirus, Porcine respirovirus, Mojiang virus, salmon aquaparamyxovirus, Cetacean morbillivirus, Reptilian Ferlavirus, and Langya henipavirus.

In certain embodiments, the class I fusion protein is a paramyxovirus F protein comprising a mutation of an amino acid residue selected from the group consisting of S, T, A and Y at a position corresponding to position 470 and/or position 477 in SEQ ID NO: 1, into a hydrophobic amino acid residue.

Several hydrophobic amino acid residues may be suitable, such as, but not limited to V, I, M and L. In a preferred embodiment, the hydrophobic amino acid is selected from the group consisting of V, I, M and L. In a preferred embodiment, the hydrophobic amino acid at the position corresponding to position 470 and at position 477 in SEQ ID NO: 1 is valine (V).

In certain embodiments, the class I fusion protein is not a PIV-3 fusion (F) protein.

In certain preferred embodiments, the paramyxovirus F protein is from Nipah virus, or human parainfluenza virus 1 (HPIV1).

In certain other embodiments, the class I fusion protein is a coronavirus S protein comprising a mutation of an amino acid residue selected from the group consisting of S, T, A and Y at a position corresponding to position 1259 and position 1266 in SEQ ID NO: 53, into a hydrophobic amino acid residue.

Several hydrophobic amino acid residues may be suitable, such as, but not limited to V, I, M, and L. In a preferred embodiment, the hydrophobic amino acid is selected from the group consisting of V, I, M and L.

In a preferred embodiment, the hydrophobic amino acid at the position corresponding to position 1259 and 1266 in SEQ ID NO; 53 is valine (V), isoleucine (I) or leucine (L).

In certain embodiments, the coronavirus S protein is an S protein from an α-coronavirus or δ-coronavirus.

According to the invention, the α-coronavirus can be any existing or future α-coronavirus, in particular any α-coronavirus with a parallel HR2 coiled-coil. In certain embodiments, the α-coronavirus is selected from the group consisting of NL63, 229E, Feline Infectious Peritonitis Virus (FIPV), Porcine Epidemic Diarrhea Virus (PEDV), Transmissible Gastroenteritis Virus (TGEV), and Canine Coronavirus (CCoV).

According to the invention, the δ-coronavirus can be any existing or future α-coronavirus, in particular any δ-coronavirus with a parallel HR2 coiled-coil. In certain embodiments, the δ-coronavirus is selected from the group consisting of HKU16, HKU19, Porcine Deltacoronavirus (PDCoV), and human Porcine Deltacoronavirus (Hu-PDCoV).

The invention also provides fragments of the class I fusion proteins. The term “fragment” as used herein refers to a class I fusion protein that has an amino-terminal (e.g. by cleaving off the signal sequence) and/or carboxy-terminal (e.g. by deleting the transmembrane region and/or cytoplasmic tail) and/or internal deletion, but wherein the remaining amino acid sequence is identical to the corresponding positions in the sequence of the class I fusion protein, for example, the full-length sequence of the class I fusion protein. It will be appreciated that for inducing an immune response and in general for vaccination purposes, a protein needs not to be full length nor have all its wild type functions, and fragments of the protein are equally useful. A fragment according to the invention is an immunologically active fragment, and typically comprises at least 15 amino acids, or at least 30 amino acids of the class I fusion protein. In certain embodiments, a fragment comprises at least 50, 75, 100, 150, 200, 250, 300, 350, 400, 450, 460, 470, 480, 490, 500, or 510 amino acids of the class I fusion protein.

In certain embodiments, the proteins or fragments thereof according to the invention do not comprise a signal sequence. It will be understood by the skilled person that signal sequences (sometimes referred to as signal peptide, targeting signal, localization signal, localization sequence, transit peptide, leader sequence or leader peptide) function to prompt a cell to translocate the protein, usually to the cellular membrane. Signal peptidase may cleave either during or after completion of translocation to generate a free signal peptide and a mature protein.

In a preferred embodiment, the fragment is a class I fusion protein ectodomain. Thus, in certain embodiments, the class I fusion protein does not comprise the transmembrane and cytoplasmic regions of the class I fusion protein. As described above, because the TM region is responsible for membrane anchoring and increases stability of the fusion protein, the ectodomain of the fusion protein is considerably more labile than the full-length protein and will even more readily refold into the post-fusion end-state.

In certain preferred embodiments, the ectodomain does not comprise a heterologous trimerization domain. HR2 stabilization according to the invention enables the provision of soluble pre-fusion trimeric class I proteins without a heterologous trimerization domain. The present invention thus provides stabilized trimeric pre-fusion class I fusion protein ectodomains without a heterologous trimerization domain.

HR2 stabilization can also prevent the conformational transition to the post-fusion state in membrane-anchored full-length proteins. This makes the approach of interest for vector- or RNA-based vaccine approaches that make use of full-length class I fusion proteins as immunogens. The present invention thus also provides stabilized trimeric full-length pre-fusion class I proteins.

According to the present invention, it has been demonstrated that trimeric paramyxovirus F ectodomain (i.e. soluble trimeric protein) of Nipah and human parainfluenza virus 1 (HPIV1) can be obtained without the presence of a heterologous trimerization domain, when the amino acid residue at a position corresponding to position 470 and/or the amino acid residue corresponding to the amino acid residue at position 477 is V, preferably when the amino acid residue at a position corresponding to position 470 and the amino acid residue corresponding to the amino acid residue at position 477 is V (wherein the numbering of the amino acid positions is according to the numbering of the amino acid residues in SEQ ID NO: 1).

In addition, it has been demonstrated that membrane-anchored full-length paramyxovirus F protein, such as Nipah F protein, cannot transition to the post-fusion conformation to mediate membrane fusion, when the amino acid residue at a position corresponding to position 470 and/or the amino acid residue at a position corresponding to position 477 is V, preferably when the amino acid residue at a position corresponding to position 470 and the amino acid residue corresponding to the amino acid residue at position 477 is V, wherein the numbering of the amino acid positions is according to the numbering of the amino acid residues in SEQ ID NO: 1.

Moreover, it has been demonstrated that trimeric NL63 coronavirus S ectodomain (i.e. soluble trimeric protein) can be obtained without the presence of a heterologous trimerization domain, when the amino acid residue at a position corresponding to position 1259 and/or the amino acid residue at a position corresponding to position 1266 is V, I, or L, wherein the numbering of the amino acid positions is according to the numbering of the amino acid residues in SEQ ID NO: 53.

In certain embodiments, the proteins comprise one or more additional mutations in the head domain of the class I fusion protein. As used herein the head domain of a class I fusion protein is defined as the ectodomain without the signal peptide and the HR2 region. The stem domain is defined here as the HR2 region.

In certain embodiments, the protein is a Nipah F protein and the one or more mutations in the head domain are a mutation of the amino acid residue at position 191 into P and/or the amino acid residue at position 452 into N.

In a preferred embodiment, the protein is a Nipah F protein comprising a mutation of the amino acid residue at position 191 into P and the amino acid residue at position 452 into N.

In certain embodiments, the protein comprises an amino acid selected from the group consisting of SEQ ID NO: 4-9, 11-13, 15, 17-19 and 24-35, or a fragment thereof.

In certain embodiments, the protein comprises an amino acid sequence selected from the group consisting of SEQ ID NO: 36-52, or a fragment thereof, wherein the amino acid residue at a position corresponding to position 470 and/or 477 in SEQ ID NO: 1, is mutated into V.

In certain embodiments, the protein comprises an amino acid sequence selected from the group consisting of SEQ ID NO: 36-52, or a fragment thereof, wherein the amino acid residue at a position corresponding to position 470 and 477 in SEQ ID NO: 1, is mutated into V.

In certain embodiments, the protein comprises an amino acid sequence selected from the group consisting of SEQ ID NO: 53-62, or a fragment thereof, wherein the amino acid residue at a position corresponding to position 1259 and/or 1266 in SEQ ID NO: 53, is mutated into V.

In certain embodiments, the protein comprises an amino acid sequence selected from the group consisting of SEQ ID NO: 53-62, or a fragment thereof, wherein the amino acid residue at a position corresponding to position 1259 and 1266 in SEQ ID NO: 53, is mutated into V.

In certain embodiments, the proteins comprise an amino acid sequence having at least 90%, preferably at least 95%, more preferably at least 97%, more preferably at least 99% amino acid sequence identity to any one of the above sequences.

In certain embodiments, the proteins do not comprise a signal sequence.

As used throughout the present application nucleotide sequences are provided from 5′ to 3′ direction, and amino acid sequences from N-terminus to C-terminus, as custom in the art.

An amino acid according to the invention can be any of the twenty naturally occurring (or ‘standard’ amino acids). The standard amino acids can be divided into several groups based on their properties. Important factors are charge, hydrophilicity or hydrophobicity, size and functional groups. These properties are important for protein structure and protein-protein interactions. Some amino acids have special properties such as cysteine, that can form covalent disulfide bonds (or disulfide bridges) to other cysteine residues, proline that induces turns of the protein backbone, and glycine that is more flexible than other amino acids. Table 1 shows the abbreviations and properties of the standard amino acids.

TABLE 1
Standard amino acids, abbreviations and properties

| | | | Side | Side chain | |
|---|---|---|---|---|---|
| | 3- | 1- | chain | charge | Hydrophilic/ |
| Amino Acid | Letter | Letter | polarity | (pH 7.4) | hydrophobic |
| alanine | Ala | A | non-polar | Neutral | hydrophobic |
| arginine | Arg | R | polar | Positive | hydrophilic |
| asparagine | Asn | N | polar | Neutral | hydrophilic |
| aspartic acid | Asp | D | polar | Negative | hydrophilic |
| cysteine | Cys | C | non-polar | Neutral | hydrophobic |
| glutamic acid | Glu | E | polar | Negative | hydrophilic |
| glutamine | Gln | Q | polar | Neutral | hydrophilic |
| glycine | Gly | G | non-polar | Neutral | hydrophobic |
| histidine | His | H | polar | positive(10%) | hydrophilic |
| | | | | neutral(90%) | |
| isoleucine | Ile | I | non-polar | Neutral | hydrophobic |
| leucine | Leu | L | non-polar | Neutral | hydrophobic |
| lysine | Lys | K | polar | Positive | hydrophilic |
| methionine | Met | M | non-polar | Neutral | hydrophobic |
| phenylalanine | Phe | F | non-polar | Neutral | hydrophobic |
| proline | Pro | P | non-polar | Neutral | hydrophobic |
| serine | Ser | S | polar | Neutral | hydrophilic |
| threonine | Thr | T | polar | Neutral | hydrophilic |
| tryptophan | Trp | W | non-polar | Neutral | hydrophobic |
| tyrosine | Tyr | Y | polar | Neutral | hydrophobic |
| valine | Val | V | non-polar | Neutral | hydrophobic |

It will be appreciated by a skilled person that the mutations can be made to the protein by routine molecular biology procedures. The mutations according to the invention preferably result in increased expression levels and/or increased stabilization of the fusion proteins compared to fusion proteins that do not comprise these mutation(s).

The present invention further provides nucleic acid molecules encoding the fusion proteins according to the invention.

The nucleic acid molecules of the invention can be in the form of RNA or in the form of DNA obtained by cloning or produced synthetically. The DNA can be double-stranded or single-stranded.

In preferred embodiments, the nucleic acid molecules encoding the proteins according to the invention are codon-optimized for expression in mammalian cells, preferably human cells. Methods of codon-optimization are known and have been described previously (e.g. WO 96/09378). A sequence is considered codon-optimized if at least one non-preferred codon as compared to a wild type sequence is replaced by a codon that is more preferred. Herein, a non-preferred codon is a codon that is used less frequently in an organism than another codon coding for the same amino acid, and a codon that is more preferred is a codon that is used more frequently in an organism than a non-preferred codon. The frequency of codon usage for a specific organism can be found in codon frequency tables, such as in http://www.kazusa.or.jp/codon. Preferably more than one non-preferred codon, preferably most or all non-preferred codons, are replaced by codons that are more preferred. Preferably the most frequently used codons in an organism are used in a codon-optimized sequence. Replacement by preferred codons generally leads to higher expression.

It will be understood by a skilled person that numerous different polynucleotides and nucleic acid molecules can encode the same protein as a result of the degeneracy of the genetic code. It is also understood that skilled persons may, using routine techniques, make nucleotide substitutions that do not affect the protein sequence encoded by the nucleic acid molecules to reflect the codon usage of any particular host organism in which the proteins are to be expressed. Therefore, unless otherwise specified, a “nucleotide sequence encoding an amino acid sequence” includes all nucleotide sequences that are degenerate versions of each other and that encode the same amino acid sequence. Nucleotide sequences that encode proteins and RNA may or may not include introns.

Nucleic acid sequences can be cloned using routine molecular biology techniques, or generated de novo by DNA synthesis, which can be performed using routine procedures by service companies having business in the field of DNA synthesis and/or molecular cloning (e.g. GeneArt, GenScripts, Invitrogen, Eurofins).

The invention also provides vectors comprising a nucleic acid molecule as described above. In certain embodiments, a nucleic acid molecule according to the invention thus is part of a vector.

In certain embodiments of the invention, the vector is an adenovirus vector. An adenovirus according to the invention belongs to the family of the Adenoviridae, and preferably is one that belongs to the genus Mastadenovirus. It can be a human adenovirus, but also an adenovirus that infects other species, including but not limited to a bovine adenovirus (e.g., bovine adenovirus 3, BAdV3), a canine adenovirus (e.g., CAdV2), a porcine adenovirus (e.g., PAdV3 or 5), or a simian adenovirus (which includes a monkey adenovirus and an ape adenovirus, such as a chimpanzee adenovirus or a gorilla adenovirus). Preferably, the adenovirus is a human adenovirus (HAdV, or AdHu), or a simian adenovirus such as chimpanzee or gorilla adenovirus (ChAd, AdCh, or SAdV), or a rhesus monkey adenovirus (RhAd). In the invention, a human adenovirus is meant if referred to as Ad without indication of species, e.g., the brief notation “Ad26” means the same as HAdV26, which is human adenovirus serotype 26. Also as used herein, the notation “rAd” means recombinant adenovirus, e.g., “rAd26” refers to recombinant human adenovirus 26.

Most advanced studies have been performed using human adenoviruses, and human adenoviruses are preferred according to certain aspects of the invention. In certain preferred embodiments, a recombinant adenovirus according to the invention is based upon a human adenovirus. In preferred embodiments, the recombinant adenovirus is based upon a human adenovirus serotype 5, 11, 26, 34, 35, 48, 49, 50, 52, etc. According to a particularly preferred embodiment of the invention, an adenovirus is a human adenovirus of serotype 26. Advantages of these serotypes include a low seroprevalence and/or low pre-existing neutralizing antibody titers in the human population, and experience with use in human subjects in clinical trials. The preparation of recombinant adenoviral vectors is well known in the art. Preparation of rAd26 vectors is described, for example, in WO 2007/104792 and in Abbink et al., (2007) Virol 81 (9): 4654-63. Exemplary genome sequences of Ad26 are found in GenBank Accession EF 153474 and in SEQ ID NO: 1 of WO 2007/104792. Examples of vectors useful for the invention for instance include those described in WO2012/082918, the disclosure of which is incorporated herein by reference in its entirety.

The adenovirus vectors useful in the invention are typically replication deficient. In these embodiments, the virus is rendered replication deficient by deletion or inactivation of regions critical to replication of the virus, such as the E1 region. The regions can be substantially deleted or inactivated by, for example, inserting a gene of interest, such as a gene encoding the stabilized pre-fusion class I fusion protein (usually linked to a promoter), or a gene encoding the pre-fusion class I fusion protein fragment (usually linked to a promoter) within the region. In some embodiments, the vectors of the invention can contain deletions in other regions, such as the E2, E3 or E4 regions, or insertions of heterologous genes linked to a promoter within one or more of these regions. For E2- and/or E4-mutated adenoviruses, generally E2- and/or E4-complementing cell lines are used to generate recombinant adenoviruses. Mutations in the E3 region of the adenovirus need not be complemented by the cell line, since E3 is not required for replication.

A packaging cell line is typically used to produce sufficient amounts of adenovirus vectors for use in the invention. A packaging cell is a cell that comprises those genes that have been deleted or inactivated in a replication deficient vector, thus allowing the virus to replicate in the cell. Suitable packaging cell lines for adenoviruses with a deletion in the E1 region include, for example, PER.C6, 911, 293, and E1 A549.

In a preferred embodiment of the invention, the vector is an adenovirus vector, and more preferably a rAd26 vector, most preferably a rAd26 vector with at least a deletion in the E1 region of the adenoviral genome, e.g. such as that described in Abbink, J Virol, 2007. 81(9): p. 4654-63, which is incorporated herein by reference. Typically, the nucleic acid sequence encoding the fusion protein is cloned into the E1 and/or the E3 region of the adenoviral genome.

Host cells comprising the nucleic acid molecules encoding the fusion proteins form also part of the invention. The fusion proteins may be produced through recombinant DNA technology involving expression of the molecules in host cells, e.g. Chinese hamster ovary (CHO) cells, tumor cell lines, BHK cells, human cell lines such as HEK293 cells, PER.C6 cells, or yeast, fungi, insect cells, and the like, or transgenic animals or plants. In certain embodiments, the cells are from a multicellular organism, in certain embodiments they are of vertebrate or invertebrate origin. In certain embodiments, the cells are mammalian cells. In certain embodiments, the cells are human cells. In general, the production of a recombinant proteins, such the fusion proteins of the invention, in a host cell comprises the introduction of a heterologous nucleic acid molecule encoding the protein in expressible format into the host cell, culturing the cells under conditions conducive to expression of the nucleic acid molecule and allowing expression of the protein in said cell. The nucleic acid molecule encoding a protein in expressible format may be in the form of an expression cassette, and usually requires sequences capable of bringing about expression of the nucleic acid, such as enhancer(s), promoter, polyadenylation signal, and the like. The person skilled in the art is aware that various promoters can be used to obtain expression of a gene in host cells. Promoters can be constitutive or regulated, and can be obtained from various sources, including viruses, prokaryotic, or eukaryotic sources, or artificially designed.

Cell culture media are available from various vendors, and a suitable medium can be routinely chosen for a host cell to express the protein of interest, here the pre-fusion class I fusion proteins. The suitable medium may or may not contain serum.

A “heterologous nucleic acid molecule” (also referred to herein as ‘transgene’) is a nucleic acid molecule that is not naturally present in the host cell. It is introduced into for instance a vector by standard molecular biology techniques. A transgene is generally operably linked to expression control sequences. This can for instance be done by placing the nucleic acid encoding the transgene(s) under the control of a promoter. Further regulatory sequences may be added. Many promoters can be used for expression of a transgene(s), and are known to the skilled person, e.g. these may comprise viral, mammalian, synthetic promoters, and the like. A non-limiting example of a suitable promoter for obtaining expression in eukaryotic cells is a CMV-promoter (U.S. Pat. No. 5,385,839), e.g. the CMV immediate early promoter, for instance comprising nt. −735 to +95 from the CMV immediate early gene enhancer/promoter. A polyadenylation signal, for example the bovine growth hormone polyA signal (U.S. Pat. No. 5,122,458), may be present behind the transgene(s). Alternatively, several widely used expression vectors are available in the art and from commercial sources, e.g. the pcDNA and pEF vector series of Invitrogen, pMSCV and pTK-Hyg from BD Sciences, pCMV-Script from Stratagene, etc, which can be used to recombinantly express the protein of interest, or to obtain suitable promoters and/or transcription terminator sequences, poly A sequences, and the like.

The cell culture can be any type of cell culture, including adherent cell culture, e.g. cells attached to the surface of a culture vessel or to microcarriers, as well as suspension culture. Most large-scale suspension cultures are operated as batch or fed-batch processes because they are the most straightforward to operate and scale up. Nowadays, continuous processes based on perfusion principles are becoming more common and are also suitable. Suitable culture media are also well known to the skilled person and can generally be obtained from commercial sources in large quantities, or custom-made according to standard protocols. Culturing can be done for instance in dishes, roller bottles or in bioreactors, using batch, fed-batch, continuous systems and the like. Suitable conditions for culturing cells are known (see e.g. Tissue Culture, Academic Press, Kruse and Paterson, editors (1973), and R. I. Freshney, Culture of animal cells: A manual of basic technique, fourth edition (Wiley-Liss Inc., 2000, ISBN 0-471-34889-9)).

The invention further provides compositions comprising a class I fusion protein, and/or fragment thereof, and/or a nucleic acid molecule, and/or a vector, as described herein. The invention thus provides compositions comprising a fusion protein, or fragment thereof. The class I fusion protein preferably is a trimeric fusion protein ectodomain without a heterologous treimerization domain. The invention also provides compositions comprising a nucleic acid molecule and/or a vector, encoding such fusion protein or fragment. The invention further provides pharmaceutical compositions, e.g. vaccine compositions, comprising a fusion protein, a fusion protein fragment, and/or a nucleic acid molecule, and/or a vector, as described above and one or more pharmaceutically acceptable excipients.

The invention also provides the use of a stabilized class I fusion protein (fragment), a nucleic acid molecule, and/or a vector, according to the invention, for inducing an immune response against said fusion protein in a subject. Further provided are methods for inducing an immune response against a class I fusion protein in a subject, comprising administering to the subject a fusion protein (fragment), and/or a nucleic acid molecule, and/or a vector, according to the invention. Also provided are class I fusion protein (fragments), nucleic acid molecules, and/or vectors, according to the invention for use in inducing an immune response against said class I fusion protein in a subject. Further provided is the use of the class I fusion protein (fragments), and/or nucleic acid molecules, and/or vectors according to the invention for the manufacture of a medicament for use in inducing an immune response against said class I fusion protein in a subject. The invention in particular provides class I fusion protein (fragments), and/or nucleic acid molecules, and/or vectors according to the invention for use as a vaccine.

The class I fusion protein (fragments), nucleic acid molecules, or vectors of the invention may be used for prevention (prophylaxis) and/or treatment of infections caused by a virus from which the fusion protein is derived. In certain embodiments, the prevention and/or treatment may be targeted at patient groups that are susceptible of such infection. Such patient groups include, but are not limited to e.g., the elderly (e.g. ≥50 years old, ≥60 years old, and preferably ≥65 years old), the young (e.g. ≤5 years old, ≤1 year old), pregnant women (for maternal immunization), and hospitalized patients and patients who have been treated with an antiviral compound but have shown an inadequate antiviral response.

The class I fusion proteins, fragments, nucleic acid molecules and/or vectors according to the invention may be used in stand-alone treatment and/or prophylaxis of a disease or condition caused by a virus from which said class I fusion protein is derived, or in combination with other prophylactic and/or therapeutic treatments, such as (existing or future) vaccines, antiviral agents and/or monoclonal antibodies.

The invention further provides methods for preventing and/or treating viral infection in a subject utilizing the class I fusion proteins or fragments thereof, nucleic acid molecules and/or vectors according to the invention. In a specific embodiment, a method for preventing and/or treating a viral infection in a subject comprises administering to a subject in need thereof an effective amount of a class I fusion protein (fragment), nucleic acid molecule and/or a vector, as described above. A therapeutically effective amount refers to an amount of a protein, nucleic acid molecule or vector, that is effective for preventing, ameliorating and/or treating a disease or condition resulting from infection by a virus from which the class I fusion protein is derived. Prevention encompasses inhibiting or reducing the spread of said virus or inhibiting or reducing the onset, development or progression of one or more of the symptoms associated with infection by said virus. Amelioration as used in herein may refer to the reduction of visible or perceptible disease symptoms, viremia, or any other measurable manifestation of said viral infection.

For administering to subjects, such as humans, the invention may employ pharmaceutical compositions comprising a class I fusion protein (fragment), a nucleic acid molecule and/or a vector as described herein, and a pharmaceutically acceptable carrier or excipient. In the present context, the term “pharmaceutically acceptable” means that the carrier or excipient, at the dosages and concentrations employed, will not cause any unwanted or harmful effects in the subjects to which they are administered. Such pharmaceutically acceptable carriers and excipients are well known in the art (see Remington's Pharmaceutical Sciences, 18th edition, A. R. Gennaro, Ed., Mack Publishing Company [1990]; Pharmaceutical Formulation Development of Peptides and Proteins, S. Frokjaer and L. Hovgaard, Eds., Taylor & Francis [2000]; and Handbook of Pharmaceutical Excipients, 3rd edition, A. Kibbe, Ed., Pharmaceutical Press [2000]). The class I fusion proteins, or nucleic acid molecules, preferably are formulated and administered as a sterile solution although it may also be possible to utilize lyophilized preparations. Sterile solutions are prepared by sterile filtration or by other methods known per se in the art. The solutions are then lyophilized or filled into pharmaceutical dosage containers. The pH of the solution generally is in the range of pH 3.0 to 9.5, e.g. pH 5.0 to 7.5. The class I fusion proteins typically are in a solution having a suitable pharmaceutically acceptable buffer, and the composition may also contain a salt. Optionally stabilizing agent may be present, such as albumin. In certain embodiments, detergent is added. In certain embodiments, the fusion proteins may be formulated into an injectable preparation.

In certain embodiments, a composition according to the invention further comprises one or more adjuvants. Adjuvants are known in the art to further increase the immune response to an applied antigenic determinant. The terms “adjuvant” and “immune stimulant” are used interchangeably herein and are defined as one or more substances that cause stimulation of the immune system. In this context, an adjuvant is used to enhance an immune response to the class I fusion proteins of the invention. Examples of suitable adjuvants include aluminium salts such as aluminium hydroxide and/or aluminium phosphate; oil-emulsion compositions (or oil-in-water compositions), including squalene-water emulsions, such as MF59 (see e.g. WO 90/14837); saponin formulations, such as for example QS21 and Immunostimulating Complexes (ISCOMS) (see e.g. U.S. Pat. No. 5,057,540; WO 90/03184, WO 96/11711, WO 2004/004762, WO 2005/002620); bacterial or microbial derivatives, examples of which are monophosphoryl lipid A (MPL), 3-O-deacylated MPL (3dMPL), CpG-motif containing oligonucleotides, ADP-ribosylating bacterial toxins or mutants thereof, such as E. coli heat labile enterotoxin LT, cholera toxin CT, and the like; eukaryotic proteins (e.g. antibodies or fragments thereof (e.g. directed against the antigen itself or CD1a, CD3, CD7, CD80) and ligands to receptors (e.g. CD40L, GMCSF, GCSF, etc), which stimulate immune response upon interaction with recipient cells. In certain embodiments the compositions of the invention comprise aluminium as an adjuvant, e.g. in the form of aluminium hydroxide, aluminium phosphate, aluminium potassium phosphate, or combinations thereof, in concentrations of 0.05-5 mg, e.g. from 0.075-1.0 mg, of aluminium content per dose.

In other embodiments, the compositions do not comprise adjuvants.

In certain embodiments, the invention provides methods for making a vaccine against a virus comprising a class I fusion protein, comprising providing a fusion protein (fragment), nucleic acid or vector according to the invention and formulating it into a pharmaceutically acceptable composition. The term “vaccine” refers to an agent or composition containing an active component effective to induce a certain degree of immunity in a subject against a certain pathogen or disease, which will result in at least a decrease (up to complete absence) of the severity, duration or other manifestation of symptoms associated with infection by the pathogen or the disease. In the present invention, the vaccine comprises an effective amount of a fusion protein (fragment) and/or a nucleic acid molecule encoding a fusion protein, and/or a vector comprising said nucleic acid molecule, which results in an effective immune response against said virus. This provides a method of preventing serious lower respiratory tract disease leading to hospitalization and the decrease in frequency of complications such as pneumonia and bronchiolitis due to viral infection and replication in a subject. The term “vaccine” according to the invention implies that it is a pharmaceutical composition, and thus typically includes a pharmaceutically acceptable diluent, carrier or excipient. It may or may not comprise further active ingredients.

Administration of the compositions according to the invention can be performed using standard routes of administration. Non-limiting embodiments i include parenteral administration, such as intradermal, intramuscular, subcutaneous, transcutaneous, or mucosal administration, e.g. intranasal, oral, and the like. In one embodiment a composition is administered by intramuscular injection. The skilled person knows the various possibilities to administer a composition, e.g. a vaccine in order to induce an immune response to the antigen(s) in the vaccine.

A subject as used herein preferably is a mammal, for instance a rodent, e.g. a mouse, a cotton rat, or a non-human-primate, or a human. Preferably, the subject is a human subject.

The invention further provides methods for making a vaccine against a virus comprising a class I fusion protein, comprising providing a recombinant human adenovirus of serotype 26 that comprises nucleic acid encoding a Class I fusion protein or fragment thereof as described herein, propagating said recombinant adenovirus in a culture of host cells, isolating and purifying the recombinant adenovirus, and bringing the recombinant adenovirus in a pharmaceutically acceptable composition. In certain embodiments, provided herein are methods of producing an adenoviral particle comprising a nucleic acid molecule encoding a fusion protein or fragment thereof (transgene). The methods comprise (a) contacting a host cell of the invention with an adenoviral vector of the invention and (b) growing the host cell under conditions wherein the adenoviral particle comprising the transgene is produced. Recombinant adenovirus can be prepared and propagated in host cells, according to well-known methods, which entail cell culture of the host cells that are infected with the adenovirus. The cell culture can be any type of cell culture, including adherent cell culture, e.g. cells attached to the surface of a culture vessel or to microcarriers, as well as suspension culture.

Most large-scale suspension cultures are operated as batch or fed-batch processes because they are the most straightforward to operate and scale up. Nowadays, continuous processes based on perfusion principles are becoming more common and are also suitable (see, e.g., WO 2010/060719, and WO 2011/098592, both incorporated by reference herein, which describe suitable methods for obtaining and purifying large amounts of recombinant adenoviruses).

The invention further provides an isolated recombinant nucleic acid that forms the genome of a recombinant human adenovirus of serotype 26 that comprises nucleic acid encoding a class I fusion protein or fragment thereof, as described herein.

In addition, the proteins of the invention may be used as diagnostic tool, for example to test the immune status of an individual by establishing whether there are antibodies in the serum of such individual capable of binding to the class I fusion protein of the invention. The invention thus also relates to an in vitro diagnostic method for detecting the presence of a viral infection in a patient said method comprising the steps of a) contacting a biological sample obtained from said patient with a protein according to the invention; and b) detecting the presence of antibody-protein complexes.

The invention is further illustrated in the following examples. The examples do not limit the invention in any way. They merely serve to clarify the invention.

### EXAMPLES

### Example 1. Stabilizing Mutations in HR2 Allow Expression of Trimeric Soluble Paramyxovirus Nipah F Protein without a Heterologous GCN4 Trimerization Domain

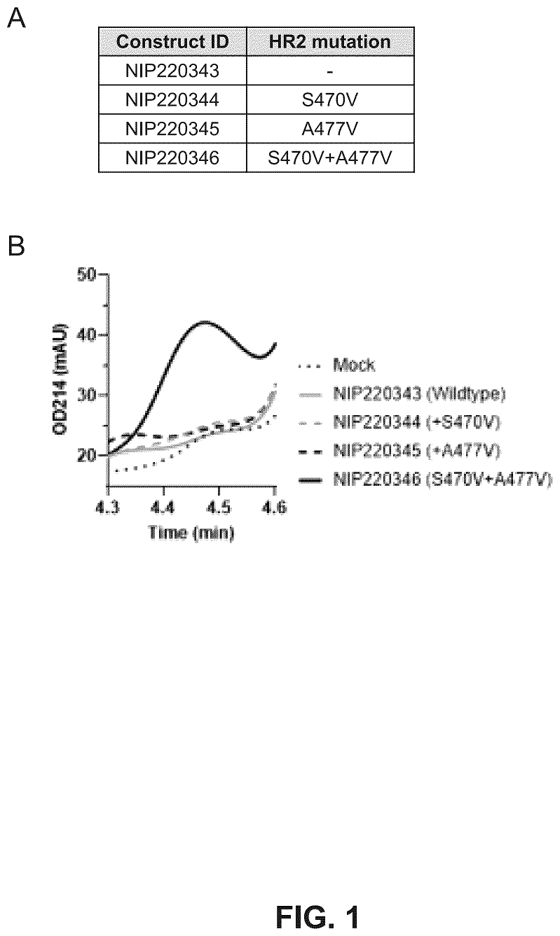

In order to stabilize the HR2 region of Nipah F and to allow Nipah F ectodomain trimer expression in the absence of a GCN4 trimerization domain, amino acid residues at position 470 and 477 were substituted in the stem region of Nipah F (NIP220355; S470V, NIP220345; A477V, NIP220346; S470V+A477V) and were compared to wild type Nipah F (NIP220343) (FIG. 1A). Plasmids coding for recombinant Nipah F protein ectodomains were equipped with a C-tag and were expressed in Expi293F cells using ExpiFectamine (Life Technologies) according to the manufacturer's instructions and cultured for 3 days at 37° C. and 10% CO2. To assess Nipah F trimer expression, sterile-filtered crude cell culture supernatant was applied to a Unix-C SEC-300 15 cm column (Sepax Technologies) with the corresponding guard column (Sepax Technologies) equilibrated in running buffer (150 mM sodium phosphate, 50 mM NaCl, pH 7.0) at 0.35 mL/min. Analytical SEC data was analyzed using Chromeleon 7.2.8.0 software package. OD214 signal of mock-transfected Expi293F cells was plotted as a baseline.

Nipah F trimer eluted at approximately 4.5 minutes retention time and was not detected upon expression of wild type or single HR2 substitution variants (NIP220343-NIP220345). In contrast, a trimer peak was detected for the double S470V+A477V variant NIP220346 (FIG. 1B), indicating that HR2 stabilization at both positions a and d of the heptad repeat (position 470 and 477 of Nipah F, respectively) is necessary and sufficient for detectable trimer expression of Nipah F protein.

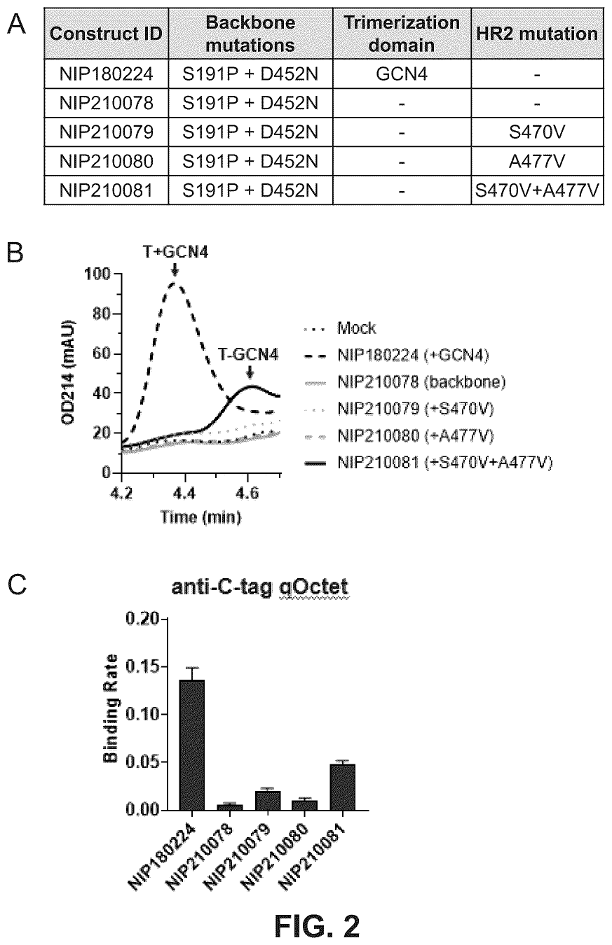

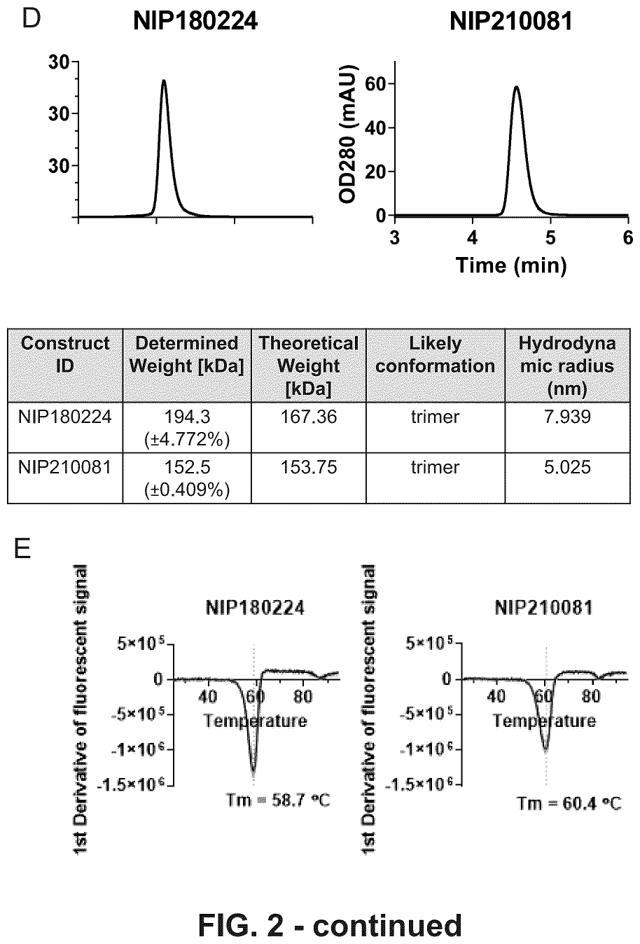

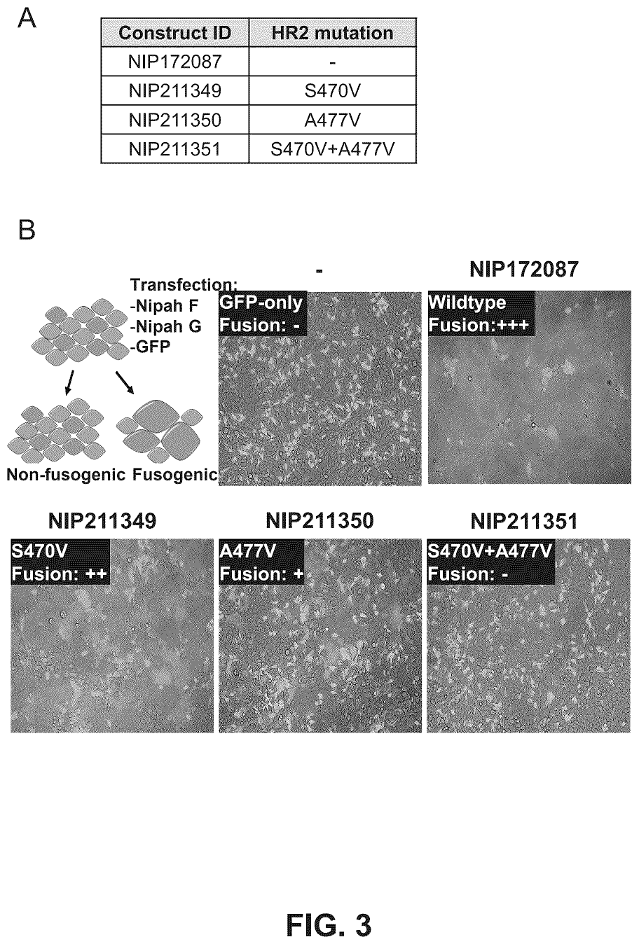

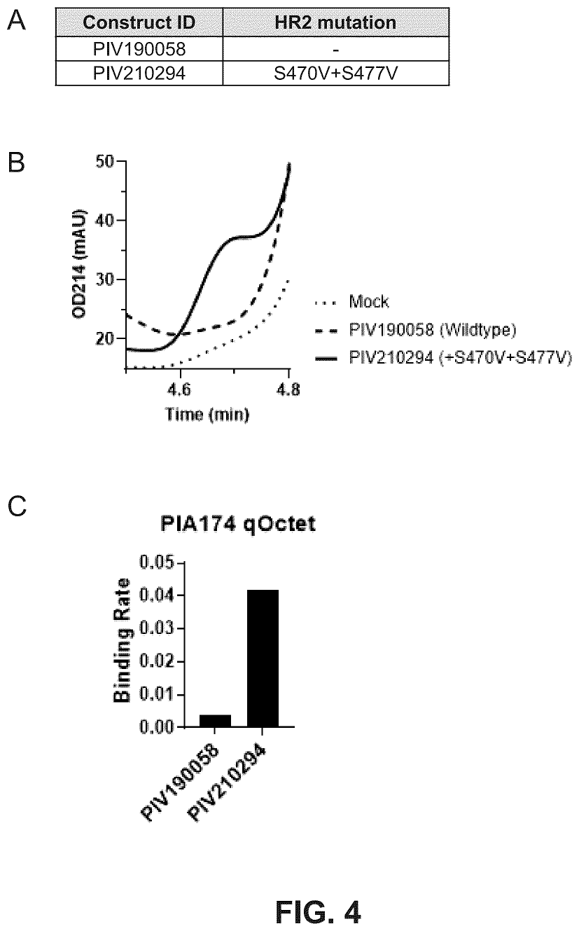

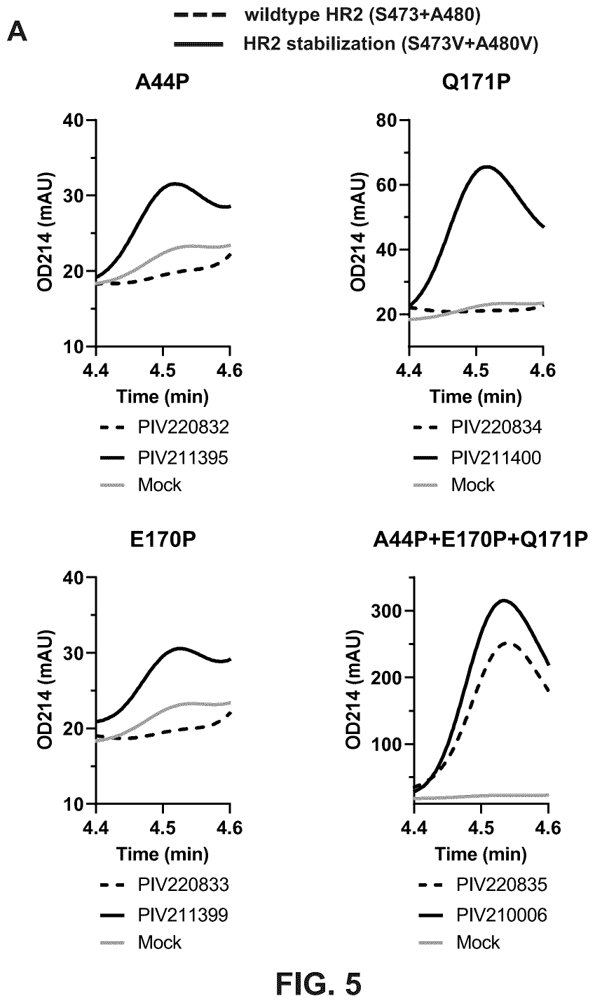

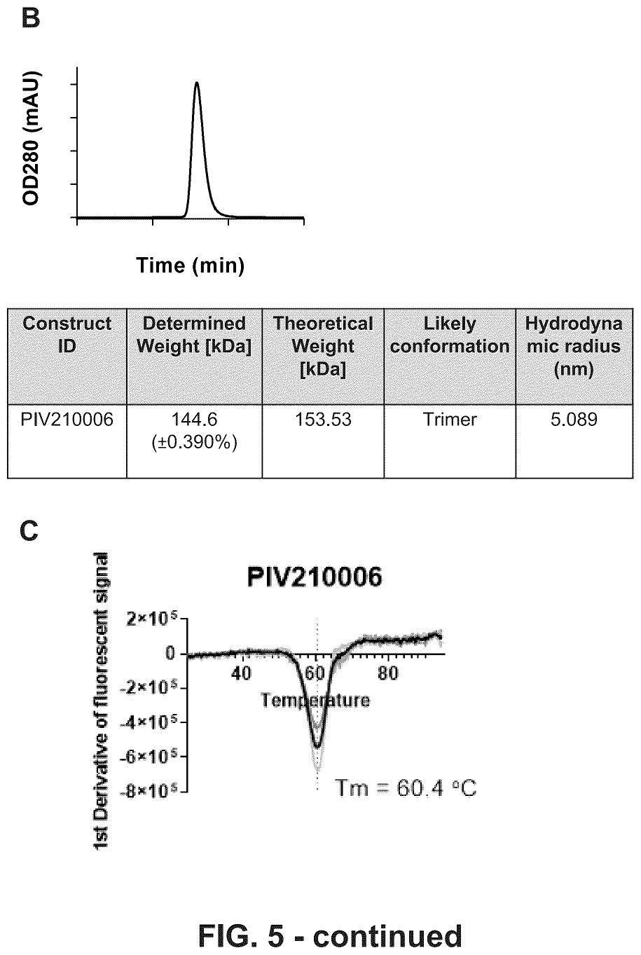

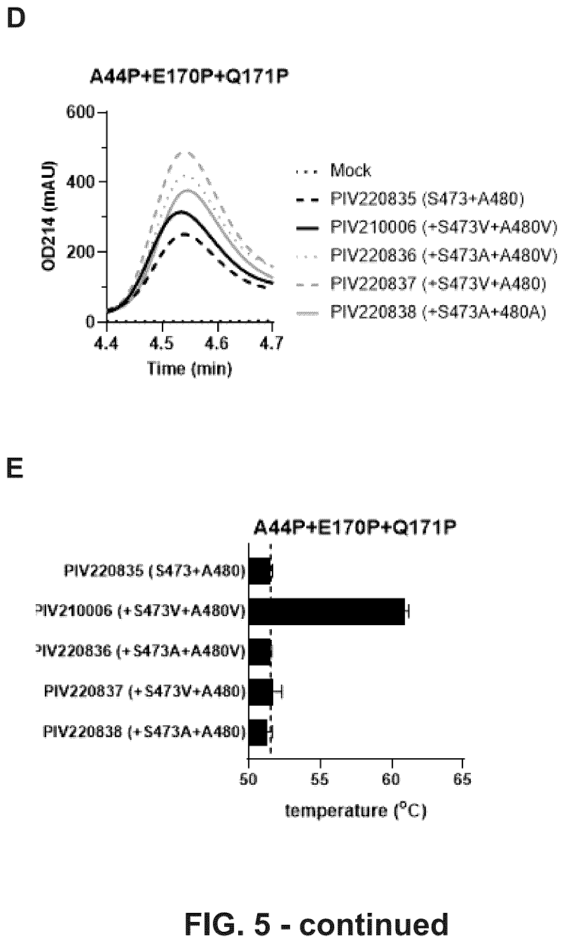

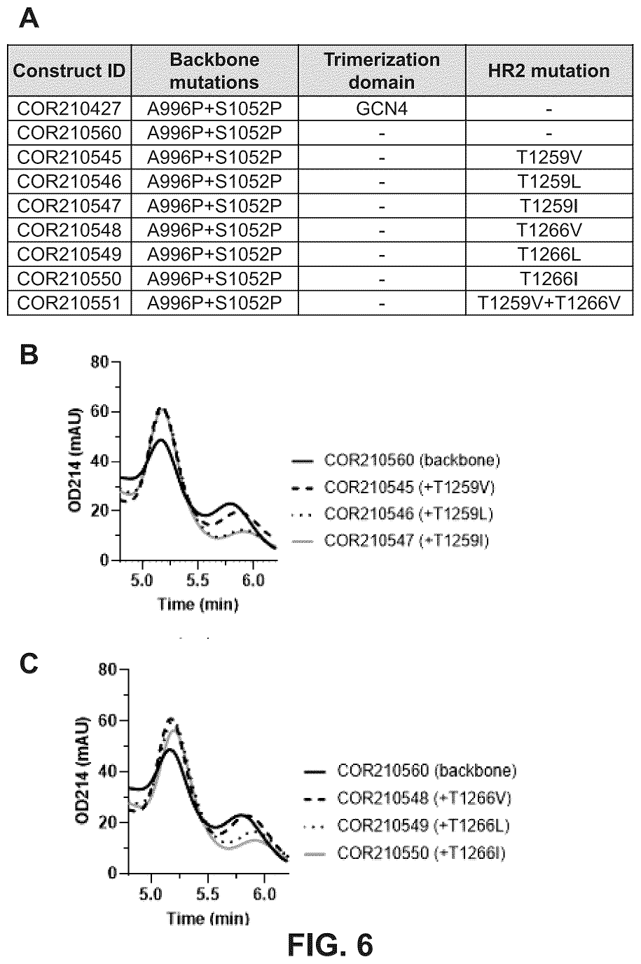

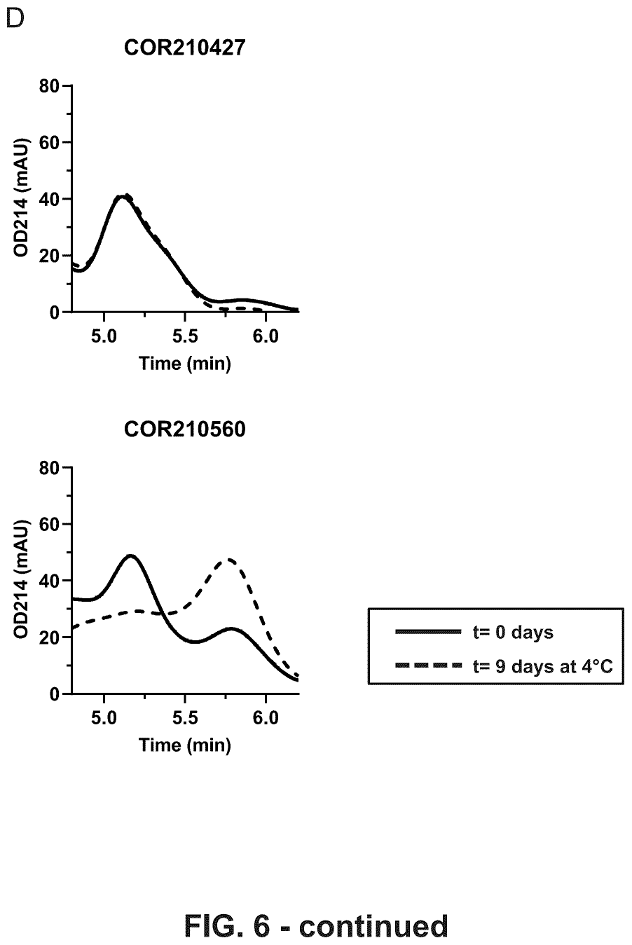

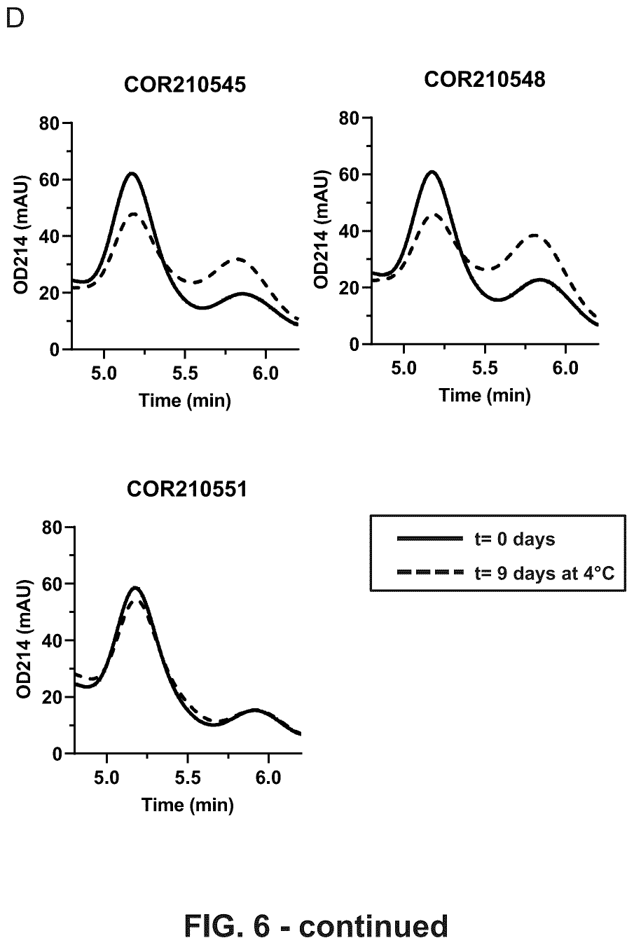

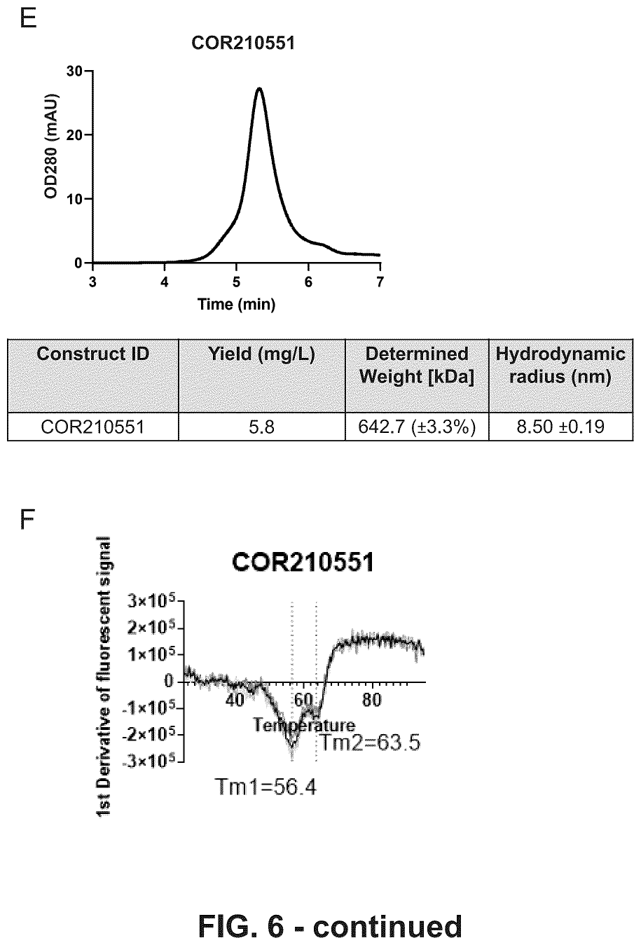

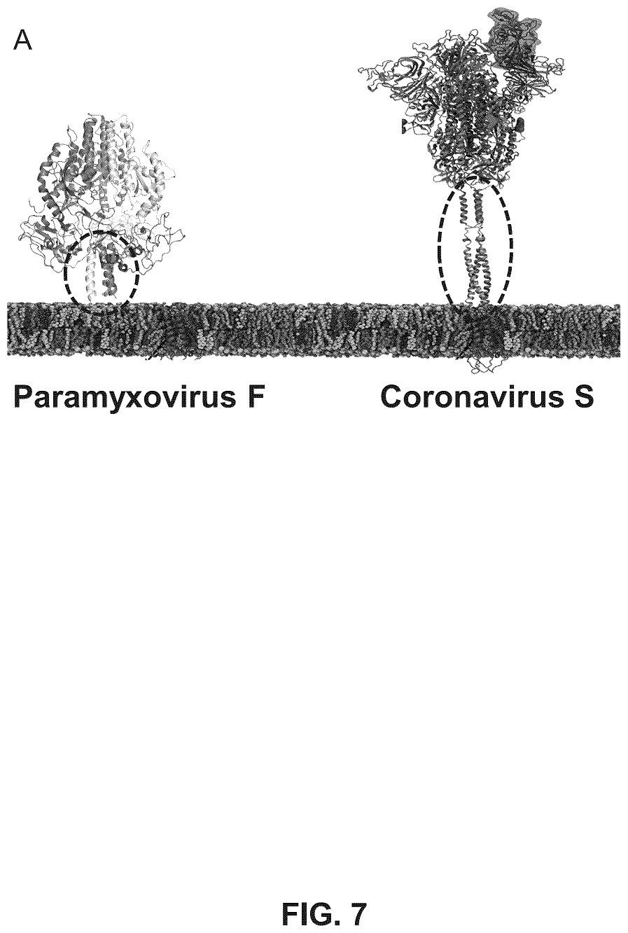

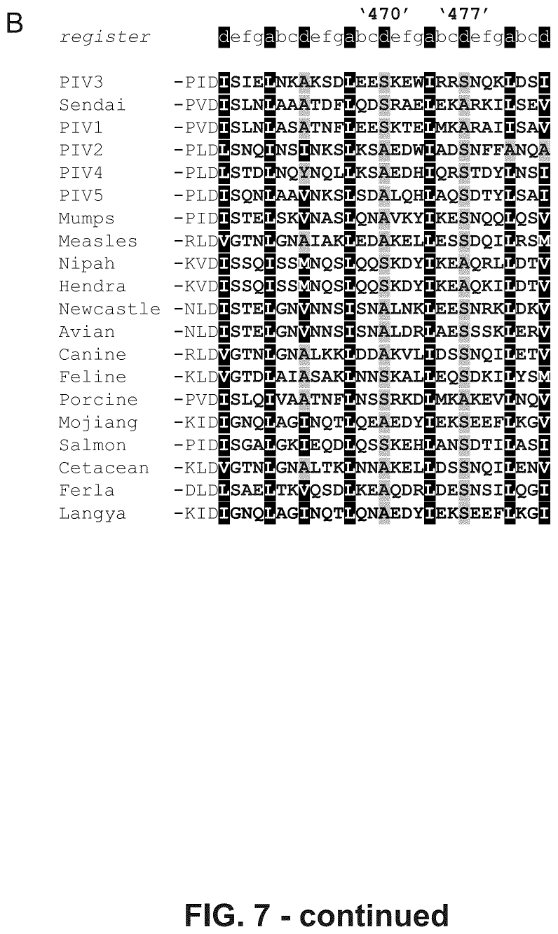

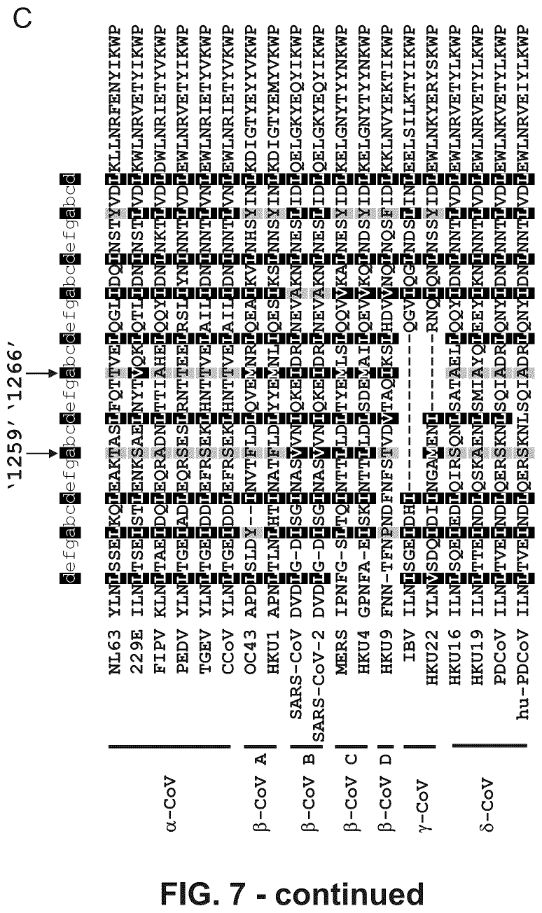

S470V and A477V were also studied in a Nipah F backbone with additional stabilizing head domain mutations, S191P and D452N (FIG. 2A). Introduction of head domain mutations alone (NIP210078) did not yield detectable trimer expression but required addition of a heterologous GCN4 trimerization domain (NIP180224) as shown by analytical SEC (FIG. 2B, indicated by T+GCN4). Removal of GCN4, while retaining trimeric Nipah F was successful only when both HR2 mutations at positions a and d of the heptad repeat were introduced (NIP210081, FIG. 2B, indicated by T−GCN4), confirming observations with wild type Nipah F backbone variants (FIG. 1B). Removal of GCN4 resulted in a longer retention time in SEC of Nipah F due to its smaller size (T+GCN4 vs T−GCN4, FIG. 2B). Expression analysis of cell culture supernatant using quantitative Octet (ForteBio Data Analysis software v12) with biotinylated anti-C-tag nanobody immobilized to streptavidin sensors confirmed highest expression of Nipah F without a trimerization domain for variant NIP210081, comprising both S470V and A477V mutations (FIG. 2C).

To further characterize Nipah F trimeric proteins, a variant with a GCN4 heterologous trimerization domain (NIP180224) and a variant without a trimerization domain, but with HR2 stabilization (NIP210081) were transiently expressed in Expi293 cells using ExpiFectamine (Life Technologies) according to the manufacturer's instructions and cultured for 5 days at 37° C. and 10% CO2. Nipah F trimer was purified from sterile-filtered crude cell culture supernatant using a two-step purification protocol including CaptureSelect™ C-tag affinity column, followed by size-exclusion chromatography using a Superdex200 10/300 column (Cytiva). The trimeric fraction was pooled and further characterized by analytical SEC-MALS using an ultra-high-performance liquid chromatography system (Vanquish, Thermo Scientific) and μDAWN TREOS instrument (Wyatt) coupled to an Optilab μT-rEX Refractive Index Detector (Wyatt), in combination with an in-line Nanostar DLS reader (Wyatt). Protein was loaded onto a Unix-C SEC-300 15 cm column (Sepax Technologies) with the corresponding guard column (Sepax Technologies) equilibrated in running buffer (150 mM sodium phosphate, 50 mM NaCl, pH 7.0) at 0.35 mL/min. Analytical SEC data was analyzed using Chromeleon 7.2.8.0 software package, and conformation, hydrodynamic radius and molecular weight of F trimers was calculated by Astra software and compared to the calculated weight, confirming a trimeric conformation (FIG. 2D).

Melting temperature (Tm₅₀) of purified Nipah F trimers was determined by differential scanning fluorimetry (DSF). To this end, the fluorescent emission of Sypro Orange Dye (ThermoFisher Scientific) added to Nipah F protein in solution was monitored. The measurement was performed with a starting temperature of 25° C. and a final temperature of 95° C. (54° C. increase per hour). Melting curves were measured using a ViiA7 real-time PCR machine (Applied Biosystems), and Tm50 values were derived from the negative first derivative as described previously (Rutten et al. (2020) Cell Rep 30:4540-4550). NIP180224 with GCN4 trimerization domain had a single melting event at 58.7° C., while HR2-stabilized NIP210081 without GCN4 trimerization domain showed a 1.7° C. increase in melting temperature with a Tm50 of 60.4° C. (FIG. 2E).

### Example 2. Stabilizing Mutations in HR2 Prevent the Conformational Transition to the Post-Fusion State in Membrane-Anchored Full-Length Paramyxovirus Nipah F Protein

To mediate cell-cell fusion, Nipah F protein undergoes a conformational change from pre-fusion to post-fusion conformation. This may be visualized by syncytia formation in cells co-expressing full-length membrane-anchored Nipah F with Nipah G and GFP reporter, to allow binding to ephrin-B2/-B3 on host cells and aid fusion (Diederich and Maisner (2007), Add N Y Acad Sci, 1102:39-50), and to visualize syncytia, respectively. 80% confluent HEK293 cell monolayers were transfected with Nipah F designs (FIG. 3A), together with Nipah G and GFP. One day post transfection, cell-cell fusion and GFP redistribution associated with syncytia formation was assessed by fluorescence microscopy. Overlays between brightfield and GFP channels were made in ImageJ and relative cell-cell fusion was scored as −/ +/ ++/+++, to signify increasing syncytia formation (FIG. 3B).

Expression of wildtype Nipah F (NIP172087) demonstrated full cell-cell fusion, compared to control cells which expressed only GFP and Nipah G (FIG. 3B). In contrast, single HR2 mutations at positions 470 (NIP211349) and 477 (NIP211350) resulted in mild to average reduction of syncytia formation, respectively (FIG. 3B). Cell-cell fusion was completely abolished when both positions were mutated (NIP211351), underscoring the importance of HR2 stabilization at positions a and d of the heptad repeat, and demonstrating that HR2 stabilization can prevent the conformational transition of pre-fusion to post-fusion Nipah F (FIG. 3B).

### Example 3. Stabilizing Mutations in HR2 Allow Expression of Trimeric Paramyxovirus HPIV3 F Protein without a Heterologous GCN4 Trimerization Domain

To stabilize the trimeric pre-fusion conformation of HPIV3 F protein ectodomain in the absence of a heterologous trimerization domain, amino acid residues at position 470 and 477 were mutated in the stem region of the HPIV3 F protein (FIG. 4A). Plasmids coding for recombinant HPIV3 F protein ectodomains which were equipped with a C-tag were expressed in Expi293Fcells, and 3 days after transfection the supernatants were analyzed for trimer content using analytical SEC and tested for binding to trimer-specific antibody PIA174 (Stewart-Jones et al. (2018) Proc Natl Acad Sci USA, 27:12265-12270) using quantitative Octet (ForteBio Data Analysis software v12) as described in example 1.

Wildtype HPIV3 F protein without GCN4 trimerization domain (PIV190058) showed no detectable trimer peak in supernatant nor binding to PIA174 (FIGS. 4B and 4C). Conversely, a detectable trimer peak and PIA174 binding was observed upon introduction of S470V+S477V HR2 stem substitutions (PIV210294), demonstrating that addition of the 470V and 477V mutation stabilized the native trimeric quaternary structure of the prefusion F protein without a GCN4 trimerization domain.

### Example 4. Stabilizing Mutations in HR2 Allow Expression of Trimeric Paramyxovirus HPIV1 F Protein without a Heterologous GCN4 Trimerization Domain

To stabilize the HR2 region of HPIV1 F and allow PIV1 F ectodomain expression in the absence of a heterologous trimerization domain, amino acid residues at position 473 and 480 in the stem region of HPIV1 F were mutated and were combined with expression-increasing mutations in the head domain of HPIV1 (FIG. 5A). Plasmids coding for recombinant HPIV1 F protein ectodomains did not carry a heterologous trimerization domain and were equipped with a C-tag and expressed in Expi293Fcells. Three days after transfection the supernatants were analyzed for trimer content using analytical SEC, as described in example 1. HPIV1 F trimer eluted at approximately 4.5 minutes retention time and was not detected upon introduction of single head domain mutations (FIG. 5A; PIV220832, PIV220833, PIV220834). However, when HR2 substitutions were added at positions 473 and 480, a detectable trimer peak was detected (FIG. 5A; PIV211395, PIV211399, PIV211400). Expression of HPIV1 trimer was considerably enhanced by the combinatorial stabilization of HR2 and three head domain mutations (FIG. 5A; PIV210006). Although HPIV1 F trimer was detected upon introduction of these three head domain mutations without HR2 stabilization (FIG. 5A; PIV220835), the temperature stability of the HR2-stabilized variant PIV210006 was almost 10° C. higher compared to its wildtype-HR2 counterpart PIV220835, underscoring the importance of HR2 stabilization for HPIV1 F trimer stability (FIG. 5E). Purification of stabilized HPIV1 F without a heterologous trimerization domain (PIV210006) and characterization of the pooled trimeric fraction was performed as described in example 1, demonstrating a single trimeric peak with expected molecular weight and a melting temperature of 60.4° C. (FIG. 5B, C).

The impact of valine (V) or alanine (A) substitutions on HR2 stabilization at positions 473 and 480 of HPIV1 F was assessed in backbone PIV220835, carrying only head domain mutations 44, 170, and 171. Each combination of V or A at either position 473 or 480 yielded increased HPIV1 F trimer expression compared to backbone PIV220835 carrying wildtype HR2 (FIG. 5D). However, only substitution by V at both positions 473+480 (PIV210006) yielded an increase in temperature stability (FIG. 5E), demonstrating the preference of V over A at positions a and d of the heptad repeat.

### Example 5. Stabilizing Mutations in HR2 Allow Expression of Trimeric Coronavirus NL63 S Protein without a Heterologous GCN4 Trimerization Domain that Remains Intact During Storage

To stabilize the HR2 region of coronavirus NL63 S and allow NL63 S ectodomain expression in the absence of a heterologous trimerization domain, amino acid residues at position 1259 and 1266 were substituted to valine (V), leucine (L), or isoleucine (I) in the stem region of NL63, as indicated in FIG. 6A, and were compared to backbone COR210560 (FIG. 6A). All plasmids coding for recombinant NL63 S protein ectodomains comprised stabilizing substitutions A996P+S1052P, did not carry a heterologous trimerization domain (except COR210427), and were equipped with a C-tag and expressed in Expi293Fcells. Three days after transfection the supernatants were analyzed for trimer content using analytical SEC, as described in example 1, with the exception of a 500-Å analytical SEC column (Sepax Technologies) instead of the 300-Å analytical SEC column (Sepax Technologies). NL63 S protein eluted as a trimeric and monomeric fraction between 5-5.5 and between 5.5-6 minutes retention time, respectively (FIG. 6B,C). Substitution of T1259 or T1266 in the HR2 region of NL63 S by either V, L, or I increased trimer expression compared to backbone COR210560 (FIG. 6B,C). However, the trimeric protein proved unstable without a GCN4 trimerization domain, as demonstrated by the appearance of monomeric S protein upon 9 days storage at 4° C. (FIG. 6D). However, when both positions were stabilized by introduction of T1259V+T1266V (COR210551), trimeric S protein was stably detected throughout the 9 days storage at 4° C., indicating that HR2 stabilization at both positions leads to more stable trimer expression of NL63 S protein (FIG. 6D).

Purification of stabilized NL63 S without a heterologous trimerization domain (COR210551) and characterization of the pooled trimeric fraction was performed as described in example 1, with the exception of using a superose 6 SEC column (Cytiva), demonstrating a single trimeric peak with expected molecular weight and a double melting event at 56.4° C. and 63.5° C. (FIG. 6E, F).

### Example 6. Identification of HR2 Repair Regions in Paramyxovirus F and Coronavirus S Proteins

Class I fusion proteins with a parallel HR2 coiled-coil include paramyxovirus F and coronavirus S proteins. HR2 stabilization in the pre-fusion conformation involves the replacement of sub-optimal (for stability of the coiled coil) amino acids (e.g. hydrophilic amino acids) on the inside of the coiled-coil into hydrophobic residues, preferably branched-chain amino acids V, I, and L. The packing (i.e. the interactions within the core of the coiled coil structure) of parallel coiled-coil regions of class I fusion proteins can be improved to generate trimers that do not need heterologous trimerization domains is encircled in FIG. 7A. To illustrate the commonality between the occurrence of suboptimal amino acid residues in the HR2 region, paramyxovirus F proteins across the phylogenetic tree were aligned and the heptad repeat register was investigated (FIG. 7B, heptad repeat shown in bold text). Optimal residues at positions a and d were highlighted in black and potential sub-optimal residues were highlighted in gray. All paramyxovirus F proteins showed potential suboptimal residues at positions 470 and 477 of the HR2 region, according to the numbering in SEQ ID NO: 1, suggesting that HR2 repair potentially negates the need for a heterologous trimerization domain across paramyxovirus F strains.

Similarly, coronavirus S proteins across the genera Alphacoronavirus (α-CoV), Betacoronavirus (β-CoV) (lineages A-D), Gammacoronavirus (γ-CoV), and Deltacoronavirus (δ-CoV) were aligned, and the heptad repeat register was investigated (FIG. 7C, heptad repeat shown in bold text). Optimal residues at positions a and d were highlighted in black and potential sub-optimal residues were highlighted in gray. All α-CoV and δ-CoV coronavirus S proteins showed potential suboptimal residues at position 1259 or 1266, or both, of the HR2 region, according to the numbering in SEQ ID NO: 53. According to the invention it has been found that HR2 stabilization can negate the need for a heterologous trimerization domain across these coronavirus genera.

SEQUENCES

SEQ ID NO: 1-NIP172087 full-length Nipah F)
MVVILDKRCYCNLLILILMISECSVGILHYEKLSKIGLVKGVTRKYKIKSNPLTKDIVI
KMIPNVSNMSQCTGSVMENYKTRLNGILTPIKGALEIYKNNTHDLVGDVRLAGVIM
AGVAIGIATAAQITAGVALYEAMKNADNINKLKSSIESTNEAVVKLQETAEKTVYVL
TALQDYINTNLVPTIDKISCKQTELSLDLALSKYLSDLLFVFGPNLQDPVSNSMTIQAI
SQAFGGNYETLLRTLGYATEDFDDLLESDSITGQIIYVDLSSYYIIVRVYFPILTEIQQA
YIQELLPVSFNNDNSEWISIVPNFILVRNTLISNIEIGFCLITKRSVICNQDYATPMTNN
MRECLTGSTEKCPRELVVSSHVPRFALSNGVLFANCISVTCQCQTTGRAISQSGEQTL
LMIDNTTCPTAVLGNVIISLGKYLGSVNYNSEGIAIGPPVFTDKVDISSQISSMNQSLQ
QSKDYIKEAQRLLDTVNPSLISMLSMIILYVLSIASLCIGLITFISFIIVEKKRNTYSRLED
RRVRPTSSGDLYYIGT
SEQ ID NO: 2-NIP180224 (Nipah F with GCN4)
MVVILDKRCYCNLLILILMISECSVGILHYEKLSKIGLVKGVTRKYKIKSNPLTKDIVI
KMIPNVSNMSQCTGSVMENYKTRLNGILTPIKGALEIYKNNTHDLVGDVRLAGVIM
AGVAIGIATAAQITAGVALYEAMKNADNINKLKSSIESTNEAVVKLQETAEKTVYVL
TALQDYINTNLVPTIDKIPCKQTELSLDLALSKYLSDLLFVFGPNLQDPVSNSMTIQAI
SQAFGGNYETLLRTLGYATEDFDDLLESDSITGQIIYVDLSSYYIIVRVYFPILTEIQQA
YIQELLPVSFNNDNSEWISIVPNFILVRNTLISNIEIGFCLITKRSVICNQDYATPMTNN
MRECLTGSTEKCPRELVVSSHVPRFALSNGVLFANCISVTCQCQTTGRAISQSGEQTL
LMIDNTTCPTAVLGNVIISLGKYLGSVNYNSEGIAIGPPVFTNKVDISSQISSMNQSLQ
QSKDYIKEAQRLLDTVNPSMKQIEDKIEEILSKIYHIENEIARIKKLIGEGGSLVPRGSG
GSEPEA
SEQ ID NO: 3-NIP210078 (Nipah F w/o GCN4)
MVVILDKRCYCNLLILILMISECSVGILHYEKLSKIGLVKGVTRKYKIKSNPLTKDIVI
KMIPNVSNMSQCTGSVMENYKTRLNGILTPIKGALEIYKNNTHDLVGDVRLAGVIM
AGVAIGIATAAQITAGVALYEAMKNADNINKLKSSIESTNEAVVKLQETAEKTVYVL
TALQDYINTNLVPTIDKIPCKQTELSLDLALSKYLSDLLFVFGPNLQDPVSNSMTIQAI
SQAFGGNYETLLRTLGYATEDFDDLLESDSITGQIIYVDLSSYYIIVRVYFPILTEIQQA
YIQELLPVSFNNDNSEWISIVPNFILVRNTLISNIEIGFCLITKRSVICNQDYATPMTNN
MRECLTGSTEKCPRELVVSSHVPRFALSNGVLFANCISVTCQCQTTGRAISQSGEQTL
LMIDNTTCPTAVLGNVIISLGKYLGSVNYNSEGIAIGPPVFTNKVDISSQISSMNQSLQ
QSKDYIKEAQRLLDTVGGSEPEA
SEQ ID NO: 4-NIP210079 (Nipah F w/o GCN4, S470V)
MVVILDKRCYCNLLILILMISECSVGILHYEKLSKIGLVKGVTRKYKIKSNPLTKDIVI
KMIPNVSNMSQCTGSVMENYKTRLNGILTPIKGALEIYKNNTHDLVGDVRLAGVIM
AGVAIGIATAAQITAGVALYEAMKNADNINKLKSSIESTNEAVVKLQETAEKTVYVL
TALQDYINTNLVPTIDKIPCKQTELSLDLALSKYLSDLLFVFGPNLQDPVSNSMTIQAI
SQAFGGNYETLLRTLGYATEDFDDLLESDSITGQIIYVDLSSYYIIVRVYFPILTEIQQA
YIQELLPVSFNNDNSEWISIVPNFILVRNTLISNIEIGFCLITKRSVICNQDYATPMTNN
MRECLTGSTEKCPRELVVSSHVPRFALSNGVLFANCISVTCQCQTTGRAISQSGEQTL
LMIDNTTCPTAVLGNVIISLGKYLGSVNYNSEGIAIGPPVFTNKVDISSQISSMNQSLQ
QvKDYIKEAQRLLDTVGGSEPEA
SEQ ID NO: 5-NIP210080 (Nipah F w/o GCN4, A477V)
MVVILDKRCYCNLLILILMISECSVGILHYEKLSKIGLVKGVTRKYKIKSNPLTKDIVI
KMIPNVSNMSQCTGSVMENYKTRLNGILTPIKGALEIYKNNTHDLVGDVRLAGVIM
AGVAIGIATAAQITAGVALYEAMKNADNINKLKSSIESTNEAVVKLQETAEKTVYVL
TALQDYINTNLVPTIDKIPCKQTELSLDLALSKYLSDLLFVFGPNLQDPVSNSMTIQAI
SQAFGGNYETLLRTLGYATEDFDDLLESDSITGQIIYVDLSSYYIIVRVYFPILTEIQQA
YIQELLPVSFNNDNSEWISIVPNFILVRNTLISNIEIGFCLITKRSVICNQDYATPMTNN
MRECLTGSTEKCPRELVVSSHVPRFALSNGVLFANCISVTCQCQTTGRAISQSGEQTL
LMIDNTTCPTAVLGNVIISLGKYLGSVNYNSEGIAIGPPVFTNKVDISSQISSMNQSLQ
QSKDYIKEvQRLLDTVGGSEPEA
SEQ ID NO: 6-NIP210081 (Nipah F w/o GCN4, S470V+A477V)
MVVILDKRCYCNLLILILMISECSVGILHYEKLSKIGLVKGVTRKYKIKSNPLTKDIVI
KMIPNVSNMSQCTGSVMENYKTRLNGILTPIKGALEIYKNNTHDLVGDVRLAGVIM
AGVAIGIATAAQITAGVALYEAMKNADNINKLKSSIESTNEAVVKLQETAEKTVYVL
TALQDYINTNLVPTIDKIPCKQTELSLDLALSKYLSDLLFVFGPNLQDPVSNSMTIQAI
SQAFGGNYETLLRTLGYATEDFDDLLESDSITGQIIYVDLSSYYIIVRVYFPILTEIQQA
YIQELLPVSFNNDNSEWISIVPNFILVRNTLISNIEIGFCLITKRSVICNQDYATPMTNN
MRECLTGSTEKCPRELVVSSHVPRFALSNGVLFANCISVTCQCQTTGRAISQSGEQTL
LMIDNTTCPTAVLGNVIISLGKYLGSVNYNSEGIAIGPPVFTNKVDISSQISSMNQSLQ
QvKDYIKEvQRLLDTVGGSEPEA
SEQ ID NO: 7-NIP211349 (Full-length Nipah F + S470V)
MVVILDKRCYCNLLILILMISECSVGILHYEKLSKIGLVKGVTRKYKIKSNPLTKDIVI
KMIPNVSNMSQCTGSVMENYKTRLNGILTPIKGALEIYKNNTHDLVGDVRLAGVIM
AGVAIGIATAAQITAGVALYEAMKNADNINKLKSSIESTNEAVVKLQETAEKTVYVL
TALQDYINTNLVPTIDKISCKQTELSLDLALSKYLSDLLFVFGPNLQDPVSNSMTIQAI
SQAFGGNYETLLRTLGYATEDFDDLLESDSITGQIIYVDLSSYYIIVRVYFPILTEIQQA
YIQELLPVSFNNDNSEWISIVPNFILVRNTLISNIEIGFCLITKRSVICNQDYATPMTNN
MRECLTGSTEKCPRELVVSSHVPRFALSNGVLFANCISVTCQCQTTGRAISQSGEQTL
LMIDNTTCPTAVLGNVIISLGKYLGSVNYNSEGIAIGPPVFTDKVDISSQISSMNQSLQ
QVKDYIKEAQRLLDTVNPSLISMLSMIILYVLSIASLCIGLITFISFIIVEKKRNTYSRLE
DRRVRPTSSGDLYYIGT
SEQ ID NO: 8-NIP211350 (Full-length Nipah F + A477V)
MVVILDKRCYCNLLILILMISECSVGILHYEKLSKIGLVKGVTRKYKIKSNPLTKDIVI
KMIPNVSNMSQCTGSVMENYKTRLNGILTPIKGALEIYKNNTHDLVGDVRLAGVIM
AGVAIGIATAAQITAGVALYEAMKNADNINKLKSSIESTNEAVVKLQETAEKTVYVL
TALQDYINTNLVPTIDKISCKQTELSLDLALSKYLSDLLFVFGPNLQDPVSNSMTIQAI
SQAFGGNYETLLRTLGYATEDFDDLLESDSITGQIIYVDLSSYYIIVRVYFPILTEIQQA
YIQELLPVSFNNDNSEWISIVPNFILVRNTLISNIEIGFCLITKRSVICNQDYATPMTNN
MRECLTGSTEKCPRELVVSSHVPRFALSNGVLFANCISVTCQCQTTGRAISQSGEQTL
LMIDNTTCPTAVLGNVIISLGKYLGSVNYNSEGIAIGPPVFTDKVDISSQISSMNQSLQ
QSKDYIKEVQRLLDTVNPSLISMLSMIILYVLSIASLCIGLITFISFIIVEKKRNTYSRLED
RRVRPTSSGDLYYIGT
SEQ ID NO: 9-NIP211351 (Full-length Nipah F + S470V+A477V)
MVVILDKRCYCNLLILILMISECSVGILHYEKLSKIGLVKGVTRKYKIKSNPLTKDIVI
KMIPNVSNMSQCTGSVMENYKTRLNGILTPIKGALEIYKNNTHDLVGDVRLAGVIM
AGVAIGIATAAQITAGVALYEAMKNADNINKLKSSIESTNEAVVKLQETAEKTVYVL
TALQDYINTNLVPTIDKISCKQTELSLDLALSKYLSDLLFVFGPNLQDPVSNSMTIQAI
SQAFGGNYETLLRTLGYATEDFDDLLESDSITGQIIYVDLSSYYIIVRVYFPILTEIQQA
YIQELLPVSFNNDNSEWISIVPNFILVRNTLISNIEIGFCLITKRSVICNQDYATPMTNN
MRECLTGSTEKCPRELVVSSHVPRFALSNGVLFANCISVTCQCQTTGRAISQSGEQTL
LMIDNTTCPTAVLGNVIISLGKYLGSVNYNSEGIAIGPPVFTDKVDISSQISSMNQSLQ
QVKDYIKEVQRLLDTVNPSLISMLSMIILYVLSIASLCIGLITFISFIIVEKKRNTYSRLE
DRRVRPTSSGDLYYIGT
SEQ ID NO: 10-NIP220343 (Soluble Nipah F Wt)
MVVILDKRCYCNLLILILMISECSVGILHYEKLSKIGLVKGVTRKYKIKSNPLTKDIVI
KMIPNVSNMSQCTGSVMENYKTRLNGILTPIKGALEIYKNNTHDLVGDVRLAGVIM
AGVAIGIATAAQITAGVALYEAMKNADNINKLKSSIESTNEAVVKLQETAEKTVYVL
TALQDYINTNLVPTIDKISCKQTELSLDLALSKYLSDLLFVFGPNLQDPVSNSMTIQAI
SQAFGGNYETLLRTLGYATEDFDDLLESDSITGQIIYVDLSSYYIIVRVYFPILTEIQQA
YIQELLPVSFNNDNSEWISIVPNFILVRNTLISNIEIGFCLITKRSVICNQDYATPMTNN
MRECLTGSTEKCPRELVVSSHVPRFALSNGVLFANCISVTCQCQTTGRAISQSGEQTL
LMIDNTTCPTAVLGNVIISLGKYLGSVNYNSEGIAIGPPVFTDKVDISSQISSMNQSLQ
QSKDYIKEAQRLLDTVGGSEPEA
SEQ ID NO: 11-NIP220344 (Soluble Nipah F S470V)
MVVILDKRCYCNLLILILMISECSVGILHYEKLSKIGLVKGVTRKYKIKSNPLTKDIVI
KMIPNVSNMSQCTGSVMENYKTRLNGILTPIKGALEIYKNNTHDLVGDVRLAGVIM
AGVAIGIATAAQITAGVALYEAMKNADNINKLKSSIESTNEAVVKLQETAEKTVYVL
TALQDYINTNLVPTIDKISCKQTELSLDLALSKYLSDLLFVFGPNLQDPVSNSMTIQAI
SQAFGGNYETLLRTLGYATEDFDDLLESDSITGQIIYVDLSSYYIIVRVYFPILTEIQQA
YIQELLPVSFNNDNSEWISIVPNFILVRNTLISNIEIGFCLITKRSVICNQDYATPMTNN
MRECLTGSTEKCPRELVVSSHVPRFALSNGVLFANCISVTCQCQTTGRAISQSGEQTL
LMIDNTTCPTAVLGNVIISLGKYLGSVNYNSEGIAIGPPVFTDKVDISSQISSMNQSLQ
QVKDYIKEAQRLLDTVGGSEPEA
SEQ ID NO: 12-NIP220345 (Soluble Nipah F A477V)
MVVILDKRCYCNLLILILMISECSVGILHYEKLSKIGLVKGVTRKYKIKSNPLTKDIVI
KMIPNVSNMSQCTGSVMENYKTRLNGILTPIKGALEIYKNNTHDLVGDVRLAGVIM
AGVAIGIATAAQITAGVALYEAMKNADNINKLKSSIESTNEAVVKLQETAEKTVYVL
TALQDYINTNLVPTIDKISCKQTELSLDLALSKYLSDLLFVFGPNLQDPVSNSMTIQAI
SQAFGGNYETLLRTLGYATEDFDDLLESDSITGQIIYVDLSSYYIIVRVYFPILTEIQQA
YIQELLPVSFNNDNSEWISIVPNFILVRNTLISNIEIGFCLITKRSVICNQDYATPMTNN
MRECLTGSTEKCPRELVVSSHVPRFALSNGVLFANCISVTCQCQTTGRAISQSGEQTL
LMIDNTTCPTAVLGNVIISLGKYLGSVNYNSEGIAIGPPVFTDKVDISSQISSMNQSLQ
QSKDYIKEVQRLLDTVGGSEPEA
SEQ ID NO: 13-NIP220346 (Soluble Nipah F S470V + A477V)
MVVILDKRCYCNLLILILMISECSVGILHYEKLSKIGLVKGVTRKYKIKSNPLTKDIVI
KMIPNVSNMSQCTGSVMENYKTRLNGILTPIKGALEIYKNNTHDLVGDVRLAGVIM
AGVAIGIATAAQITAGVALYEAMKNADNINKLKSSIESTNEAVVKLQETAEKTVYVL
TALQDYINTNLVPTIDKISCKQTELSLDLALSKYLSDLLFVFGPNLQDPVSNSMTIQAI
SQAFGGNYETLLRTLGYATEDFDDLLESDSITGQIIYVDLSSYYIIVRVYFPILTEIQQA
YIQELLPVSFNNDNSEWISIVPNFILVRNTLISNIEIGFCLITKRSVICNQDYATPMTNN
MRECLTGSTEKCPRELVVSSHVPRFALSNGVLFANCISVTCQCQTTGRAISQSGEQTL
LMIDNTTCPTAVLGNVIISLGKYLGSVNYNSEGIAIGPPVFTDKVDISSQISSMNQSLQ
QVKDYIKEVQRLLDTVGGSEPEA
SEQ ID NO: 14-PIV190058 (PIV3 w/o GCN4)
MPISILLIITTMIMASHCQIDITKLQHVGVLVNSPKGMKISQNFETRYLILSLIPKIEDSN
SCGDQQIKQYKRLLDRLIIPLYDGLRLQKDVIVTNQESNENTDPRTERFFGGVIGTIAL
GVATSAQITAAVALVEAKQARSDIEKLKEAIRDTNKAVQSVQSSVGNLIVAIKSVQD
YVNKEIVPSIARLGCEAAGLQLGIALTQHYSELTNIFGDNIGSLQEKGIKLQGIASLYR
TNITEIFTTSTVDKYDIYDLLFTESIKVRVIDVDLNDYSITLQVRLPLLTRLLNTQIYKV
DSISYNIQNREWYIPLPSHIMTKGAFLGGADVKECIEAFSSYICPSDPGFVLNHEMESC
LSGNISQCPRTTVTSDIVPRYAFVNGGVVANCITTTCTCNGIGNRINQPPDQGVKIITH
KECNTIGINGMLFNTNKEGTLAFYTPDDITLNNSVALDPIDISIELNKAKSDLEESKEW
IRRSNQKLDSIGGSEPEA
SEQ ID NO: 15-PIV210006(PIV1 F A44P + E170P + Q171P + S473V + A480V)
MQSSEILILAYSSLLLSSSLCQIPVDKLSNVGVIINEGKLLKIpGSYESRYIVLSLVPSIDL
QDGCGTTQIIQYKNLLNRLLIPLKDALDLQESLITITNDTTVTNDNPQTRFFGAVIGTIA
LGVATAAQITAGIALAEAREARKDIALIKDSIVKTHNSVEFIQRGIGppIIALKTLQDFV
NDEIRPAIGELRCETTALKLGIKLTQHYSELATAFSSNLGTIGEKSLTLQALSSLYSANI
TEILSTIKKDKSDIYDIIYTEQVKGTVIDVDLEKYMVTLLVKIPILSEIPGVLIYRASSISY
NIEGEEWHVAIPNYIISKASSLGGADVTNCIESKLAYICPRDPTQLIPDNQQKCILGDV
SKCPVTKVINNLVPKFAFINGGVVANCIASTCTCGTNRIPVNQDRSKGVTFLTYTNCG
LIGINGIELYANKRGRDTTWGNQIIKVGPAVSIRPVDISLNLASATNFLEEvKTELMKv
RAIISAVGSGEPEA
SEQ ID NO: 16-PIV210294 (PIV3 F S470V + S477V)
MPISILLIITTMIMASHCQIDITKLQHVGVLVNSPKGMKISQNFETRYLILSLIPKIEDSN
SCGDQQIKQYKRLLDRLIIPLYDGLRLQKDVIVTNQESNENTDPRTERFFGGVIGTIAL
GVATSAQITAAVALVEAKQARSDIEKLKEAIRDTNKAVQSVQSSVGNLIVAIKSVQD
YVNKEIVPSIARLGCEAAGLQLGIALTQHYSELTNIFGDNIGSLQEKGIKLQGIASLYR
TNITEIFTTSTVDKYDIYDLLFTESIKVRVIDVDLNDYSITLQVRLPLLTRLLNTQIYKV
DSISYNIQNREWYIPLPSHIMTKGAFLGGADVKECIEAFSSYICPSDPGFVLNHEMESC
LSGNISQCPRTTVTSDIVPRYAFVNGGVVANCITTTCTCNGIGNRINQPPDQGVKIITH
KECNTIGINGMLFNTNKEGTLAFYTPDDITLNNSVALDPIDISIELNKAKSDLEEvKEW
IRRvNQKLDSIGGSEPEA
SEQ ID NO: 17-PIV211395 (PIV1 FS473V + A480V + A44P)
MQSSEILILAYSSLLLSSSLCQIPVDKLSNVGVIINEGKLLKIpGSYESRYIVLSLVPSIDL
QDGCGTTQIIQYKNLLNRLLIPLKDALDLQESLITITNDTTVTNDNPQTRFFGAVIGTIA
LGVATAAQITAGIALAEAREARKDIALIKDSIVKTHNSVEFIQRGIGEQIIALKTLQDFV
NDEIRPAIGELRCETTALKLGIKLTQHYSELATAFSSNLGTIGEKSLTLQALSSLYSANI
TEILSTIKKDKSDIYDIIYTEQVKGTVIDVDLEKYMVTLLVKIPILSEIPGVLIYRASSISY
NIEGEEWHVAIPNYIISKASSLGGADVTNCIESKLAYICPRDPTQLIPDNQQKCILGDV
SKCPVTKVINNLVPKFAFINGGVVANCIASTCTCGTNRIPVNQDRSKGVTFLTYTNCG
LIGINGIELYANKRGRDTTWGNQIIKVGPAVSIRPVDISLNLASATNFLEEvKTELMKv
RAIISAVGSGEPEA
SEQ ID NO: 18-PIV211399(PIV1 F S473V + A480V + E170P)
MQSSEILILAYSSLLLSSSLCQIPVDKLSNVGVIINEGKLLKIAGSYESRYIVLSLVPSID
LQDGCGTTQIIQYKNLLNRLLIPLKDALDLQESLITITNDTTVTNDNPQTRFFGAVIGTI
ALGVATAAQITAGIALAEAREARKDIALIKDSIVKTHNSVEFIQRGIGpQIIALKTLQDF
VNDEIRPAIGELRCETTALKLGIKLTQHYSELATAFSSNLGTIGEKSLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEvKTELM
KvRAIISAVGSGEPEA
SEQ ID NO: 19-PIV211400 (PIV1 FS473V + A480V + Q171P)
MQSSEILILAYSSLLLSSSLCQIPVDKLSNVGVIINEGKLLKIAGSYESRYIVLSLVPSID
LQDGCGTTQIIQYKNLLNRLLIPLKDALDLQESLITITNDTTVTNDNPQTRFFGAVIGTI
ALGVATAAQITAGIALAEAREARKDIALIKDSIVKTHNSVEFIQRGIGEpIIALKTLQDF
VNDEIRPAIGELRCETTALKLGIKLTQHYSELATAFSSNLGTIGEKSLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEEvKTELM
KvRAIISAVGSGEPEA
SEQ ID NO: 20-PIV220832 (PIV1 F A44P)
MQSSEILILAYSSLLLSSSLCQIPVDKLSNVGVIINEGKLLKIPGSYESRYIVLSLVPSIDL
QDGCGTTQIIQYKNLLNRLLIPLKDALDLQESLITITNDTTVINDNPQTRFFGAVIGTIA
LGVATAAQITAGIALAEAREARKDIALIKDSIVKTHNSVEFIQRGIGEQIIALKTLQDFV
NDEIRPAIGELRCETTALKLGIKLTQHYSELATAFSSNLGTIGEKSLTLQALSSLYSANI
TEILSTIKKDKSDIYDIIYTEQVKGTVIDVDLEKYMVTLLVKIPILSEIPGVLIYRASSISY
NIEGEEWHVAIPNYIISKASSLGGADVTNCIESKLAYICPRDPTQLIPDNQQKCILGDV
SKCPVTKVINNLVPKFAFINGGVVANCIASTCTCGTNRIPVNQDRSKGVTFLTYTNCG
LIGINGIELYANKRGRDTTWGNQIIKVGPAVSIRPVDISLNLASATNFLEESKTELMKA
RAIISAVGSGEPEA
SEQ ID NO: 21-PIV220833 (PIV1 F E170P)
MQSSEILILAYSSLLLSSSLCQIPVDKLSNVGVIINEGKLLKIAGSYESRYIVLSLVPSID
LQDGCGTTQIIQYKNLLNRLLIPLKDALDLQESLITITNDTTVTNDNPQTRFFGAVIGTI
ALGVATAAQITAGIALAEAREARKDIALIKDSIVKTHNSVEFIQRGIGPQIIALKTLQDF
VNDEIRPAIGELRCETTALKLGIKLTQHYSELATAFSSNLGTIGEKSLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVINCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEESKTELM
KARAIISAVGSGEPEA
SEQ ID NO: 22-PIV220834 (PIV1 F Q171P)
MQSSEILILAYSSLLLSSSLCQIPVDKLSNVGVIINEGKLLKIAGSYESRYIVLSLVPSID
LQDGCGTTQIIQYKNLLNRLLIPLKDALDLQESLITITNDTTVTNDNPQTRFFGAVIGTI
ALGVATAAQITAGIALAEAREARKDIALIKDSIVKTHNSVEFIQRGIGEPIIALKTLQDF
VNDEIRPAIGELRCETTALKLGIKLTQHYSELATAFSSNLGTIGEKSLTLQALSSLYSA
NITEILSTIKKDKSDIYDIIYTEQVKGTVIDVDLEKYMVTLLVKIPILSEIPGVLIYRASSI
SYNIEGEEWHVAIPNYIISKASSLGGADVTNCIESKLAYICPRDPTQLIPDNQQKCILG
DVSKCPVTKVINNLVPKFAFINGGVVANCIASTCTCGTNRIPVNQDRSKGVTFLTYTN
CGLIGINGIELYANKRGRDTTWGNQIIKVGPAVSIRPVDISLNLASATNFLEESKTELM
KARAIISAVGSGEPEA
SEQ ID NO: 23-PIV220835 (PIV1 F A44P + E170P + Q171P)
MQSSEILILAYSSLLLSSSLCQIPVDKLSNVGVIINEGKLLKIPGSYESRYIVLSLVPSIDL
QDGCGTTQIIQYKNLLNRLLIPLKDALDLQESLITITNDTTVINDNPQTRFFGAVIGTIA
LGVATAAQITAGIALAEAREARKDIALIKDSIVKTHNSVEFIQRGIGPPIIALKTLQDFV
NDEIRPAIGELRCETTALKLGIKLTQHYSELATAFSSNLGTIGEKSLTLQALSSLYSANI
TEILSTIKKDKSDIYDIIYTEQVKGTVIDVDLEKYMVTLLVKIPILSEIPGVLIYRASSISY
NIEGEEWHVAIPNYIISKASSLGGADVTNCIESKLAYICPRDPTQLIPDNQQKCILGDV
SKCPVTKVINNLVPKFAFINGGVVANCIASTCTCGTNRIPVNQDRSKGVTFLTYTNCG
LIGINGIELYANKRGRDTTWGNQIIKVGPAVSIRPVDISLNLASATNFLEESKTELMKA
RAIISAVGSGEPEA
SEQ ID NO: 24-PIV220836
MQSSEILILAYSSLLLSSSLCQIPVDKLSNVGVIINEGKLLKIpGSYESRYIVLSLVPSIDL
QDGCGTTQIIQYKNLLNRLLIPLKDALDLQESLITITNDTTVTNDNPQTRFFGAVIGTIA
LGVATAAQITAGIALAEAREARKDIALIKDSIVKTHNSVEFIQRGIGppIIALKTLQDFV
NDEIRPAIGELRCETTALKLGIKLTQHYSELATAFSSNLGTIGEKSLTLQALSSLYSANI
TEILSTIKKDKSDIYDIIYTEQVKGTVIDVDLEKYMVTLLVKIPILSEIPGVLIYRASSISY
NIEGEEWHVAIPNYIISKASSLGGADVTNCIESKLAYICPRDPTQLIPDNQQKCILGDV
SKCPVTKVINNLVPKFAFINGGVVANCIASTCTCGTNRIPVNQDRSKGVTFLTYTNCG
LIGINGIELYANKRGRDTTWGNQIIKVGPAVSIRPVDISLNLASATNFLEEAKTELMKV
RAIISAVGSGEPEA
SEQ ID NO: 25-PIV220837
MQSSEILILAYSSLLLSSSLCQIPVDKLSNVGVIINEGKLLKIpGSYESRYIVLSLVPSIDL
QDGCGTTQIIQYKNLLNRLLIPLKDALDLQESLITITNDTTVINDNPQTRFFGAVIGTIA
LGVATAAQITAGIALAEAREARKDIALIKDSIVKTHNSVEFIQRGIGppIIALKTLQDFV
NDEIRPAIGELRCETTALKLGIKLTQHYSELATAFSSNLGTIGEKSLTLQALSSLYSANI
TEILSTIKKDKSDIYDIIYTEQVKGTVIDVDLEKYMVTLLVKIPILSEIPGVLIYRASSISY
NIEGEEWHVAIPNYIISKASSLGGADVTNCIESKLAYICPRDPTQLIPDNQQKCILGDV
SKCPVTKVINNLVPKFAFINGGVVANCIASTCTCGTNRIPVNQDRSKGVTFLTYTNCG
LIGINGIELYANKRGRDTTWGNQIIKVGPAVSIRPVDISLNLASATNFLEEvKTELMKA
RAIISAVGSGEPEA
SEQ ID NO: 26-PIV220838
MQSSEILILAYSSLLLSSSLCQIPVDKLSNVGVIINEGKLLKIpGSYESRYIVLSLVPSIDL
QDGCGTTQIIQYKNLLNRLLIPLKDALDLQESLITITNDTTVINDNPQTRFFGAVIGTIA
LGVATAAQITAGIALAEAREARKDIALIKDSIVKTHNSVEFIQRGIGppIIALKTLQDFV
NDEIRPAIGELRCETTALKLGIKLTQHYSELATAFSSNLGTIGEKSLTLQALSSLYSANI
TEILSTIKKDKSDIYDIIYTEQVKGTVIDVDLEKYMVTLLVKIPILSEIPGVLIYRASSISY
NIEGEEWHVAIPNYIISKASSLGGADVTNCIESKLAYICPRDPTQLIPDNQQKCILGDV
SKCPVTKVINNLVPKFAFINGGVVANCIASTCTCGTNRIPVNQDRSKGVTFLTYTNCG
LIGINGIELYANKRGRDTTWGNQIIKVGPAVSIRPVDISLNLASATNFLEEAKTELMKA
RAIISAVGSGEPEA
SEQ ID NO: 27-COR210427
MKLFLILLVLPLASCFFTCNSNANLSMLQLGVPDNSSTIVTGLLPTHWICANQSTSVY
SANGFFYIDVGNHRSAFALHTGYYDVNQYYIYVTNEIGLNASVTLKICKFGINTTFDF
LSNSSSSFDCIVNLLFTEQLGAPLGITISGETVRLHLYNVTRTFYVPAAYKLTKLSVKC
YFNYSCVFSVVNATVTVNVTTHNGRVVNYTVCDDCNGYTDNIFSVQQDGRIPNGFP
FNNWFLLTNDSTLVDGVSRLYQPLRLTCLWPVPGLKSSTGFVYFNATGSDVNCNGY
QHNSVADVMRYNLNFSANSVDNLKSGVIVFKTLQYDVLFYCSNSSSGVLDTTIPFGP
SSQPYYCFINSTINTTHVSTFVGVLPPTVREIVVARTGQFYINGFKYFDLGFIEAVNFN
VTTASATDFWTVAFATFVDVLVNVSATKIQNLLYCDSPFEKLQCEHLQFGLQDGFYS
ANFLDDNVLPETYVALPIYYQHTDINFTATASFGGSCYVCKPHQVNISLNGNTSVCV
RTSHFSIRYIYNRVKSGSPGDSSWHIYLKSGTCPFSFSKLNNFQKFKTICFSTVAVPGS
CNFPLEATWHYTSYTIVGALYVTWSEGNSITGVPYPVSGIREFSNLVLNNCTKYNIYD
YVGTGIIRSSNQSLAGGITYVSNSGNLLGFKNVSTGNIFIVTPCNQPDQVAVYQQSIIG
AMTAVNESRYGLQNLLQLPNFYYVSNGGNNCTTAVMTYSNFGICADGSLIPVRPRN
SSDNGISAIITANLSIPSNWTTSVQVEYLQITSTPIVVDCATYVCNGNPRCKNLLKQYT
SACKTIEDALRLSAHLETNDVSSMLTFDSNAFSLANVTSFGDYNLSSVLPQRNIHSSRI
AGRSALEDLLFSKVVTSGLGTVDVDYKSCTKGLSIADLACAQYYNGIMVLPGVADA
ERMAMYTGSLIGGMVLGGLTSAAAIPFSLALQARLNYVALQTDVLQENQKILAASF
NKAINNIVASFSSVNDPITQTAEAIHTVTIALNKIQDVVNQQGSALNHLTSQLRHNFQ
AISNSIQAIYDRLDPIQADQQVDRLITGRLAALNAFVSQVLNKYTEVRSSRRLAQQKI
NECVKSQSNRYGFCGNGTHIFSIVNSAPDGLLFLHTVLLPTDYKNVKAWSGICVDGI
YGYVLRQPNLVLYSDNGVFRVTSRVMFQPRLPVLSDFVQIYNCNVTFVNISRVELHT
VIPDYVDVNKTLQEFAQNLPKYVKPNFDLTPFNLTYLNLSSELKQLEAKTASLFQTT
VELQGLIDQINSTYVDLEDKIEEILSKIYHIENEIARIKKLIGEAGSGEPEA
SEQ ID NO: 28-COR210545
MKLFLILLVLPLASCFFTCNSNANLSMLQLGVPDNSSTIVTGLLPTHWICANQSTSVY
SANGFFYIDVGNHRSAFALHTGYYDVNQYYIYVTNEIGLNASVTLKICKFGINTTFDF
LSNSSSSFDCIVNLLFTEQLGAPLGITISGETVRLHLYNVTRTFYVPAAYKLTKLSVKC
YFNYSCVFSVVNATVTVNVTTHNGRVVNYTVCDDCNGYTDNIFSVQQDGRIPNGFP
FNNWFLLTNDSTLVDGVSRLYQPLRLTCLWPVPGLKSSTGFVYFNATGSDVNCNGY
QHNSVADVMRYNLNFSANSVDNLKSGVIVFKTLQYDVLFYCSNSSSGVLDTTIPFGP
SSQPYYCFINSTINTTHVSTFVGVLPPTVREIVVARTGQFYINGFKYFDLGFIEAVNFN
VTTASATDFWTVAFATFVDVLVNVSATKIQNLLYCDSPFEKLQCEHLQFGLQDGFYS
ANFLDDNVLPETYVALPIYYQHTDINFTATASFGGSCYVCKPHQVNISLNGNTSVCV
RTSHFSIRYIYNRVKSGSPGDSSWHIYLKSGTCPFSFSKLNNFQKFKTICFSTVAVPGS
CNFPLEATWHYTSYTIVGALYVTWSEGNSITGVPYPVSGIREFSNLVLNNCTKYNIYD
YVGTGIIRSSNQSLAGGITYVSNSGNLLGFKNVSTGNIFIVTPCNQPDQVAVYQQSIIG
AMTAVNESRYGLQNLLQLPNFYYVSNGGNNCTTAVMTYSNFGICADGSLIPVRPRN
SSDNGISAIITANLSIPSNWTTSVQVEYLQITSTPIVVDCATYVCNGNPRCKNLLKQYT
SACKTIEDALRLSAHLETNDVSSMLTFDSNAFSLANVTSFGDYNLSSVLPQRNIHSSRI
AGRSALEDLLFSKVVTSGLGTVDVDYKSCTKGLSIADLACAQYYNGIMVLPGVADA
ERMAMYTGSLIGGMVLGGLTSAAAIPFSLALQARLNYVALQTDVLQENQKILAASF
NKAINNIVASFSSVNDPITQTAEAIHTVTIALNKIQDVVNQQGSALNHLTSQLRHNFQ
AISNSIQAIYDRLDPIQADQQVDRLITGRLAALNAFVSQVLNKYTEVRSSRRLAQQKI
NECVKSQSNRYGFCGNGTHIFSIVNSAPDGLLFLHTVLLPTDYKNVKAWSGICVDGI
YGYVLRQPNLVLYSDNGVFRVTSRVMFQPRLPVLSDFVQIYNCNVTFVNISRVELHT
VIPDYVDVNKTLQEFAQNLPKYVKPNFDLTPFNLTYLNLSSELKQLEAKVASLFQTT
VELQGLIDQINSTYVDLGSGEPEA
SEQ ID NO: 29-COR210546
MKLFLILLVLPLASCFFTCNSNANLSMLQLGVPDNSSTIVTGLLPTHWICANQSTSVY
SANGFFYIDVGNHRSAFALHTGYYDVNQYYIYVTNEIGLNASVTLKICKFGINTTFDF
LSNSSSSFDCIVNLLFTEQLGAPLGITISGETVRLHLYNVTRTFYVPAAYKLTKLSVKC
YFNYSCVFSVVNATVTVNVTTHNGRVVNYTVCDDCNGYTDNIFSVQQDGRIPNGFP
FNNWFLLTNDSTLVDGVSRLYQPLRLTCLWPVPGLKSSTGFVYFNATGSDVNCNGY
QHNSVADVMRYNLNFSANSVDNLKSGVIVFKTLQYDVLFYCSNSSSGVLDTTIPFGP
SSQPYYCFINSTINTTHVSTFVGVLPPTVREIVVARTGQFYINGFKYFDLGFIEAVNFN
VTTASATDFWTVAFATFVDVLVNVSATKIQNLLYCDSPFEKLQCEHLQFGLQDGFYS
ANFLDDNVLPETYVALPIYYQHTDINFTATASFGGSCYVCKPHQVNISLNGNTSVCV
RTSHFSIRYIYNRVKSGSPGDSSWHIYLKSGTCPFSFSKLNNFQKFKTICFSTVAVPGS
CNFPLEATWHYTSYTIVGALYVTWSEGNSITGVPYPVSGIREFSNLVLNNCTKYNIYD
YVGTGIIRSSNQSLAGGITYVSNSGNLLGFKNVSTGNIFIVTPCNQPDQVAVYQQSIIG
AMTAVNESRYGLQNLLQLPNFYYVSNGGNNCTTAVMTYSNFGICADGSLIPVRPRN
SSDNGISAIITANLSIPSNWTTSVQVEYLQITSTPIVVDCATYVCNGNPRCKNLLKQYT
SACKTIEDALRLSAHLETNDVSSMLTFDSNAFSLANVTSFGDYNLSSVLPQRNIHSSRI
AGRSALEDLLFSKVVTSGLGTVDVDYKSCTKGLSIADLACAQYYNGIMVLPGVADA
ERMAMYTGSLIGGMVLGGLTSAAAIPFSLALQARLNYVALQTDVLQENQKILAASF
NKAINNIVASFSSVNDPITQTAEAIHTVTIALNKIQDVVNQQGSALNHLTSQLRHNFQ
AISNSIQAIYDRLDPIQADQQVDRLITGRLAALNAFVSQVLNKYTEVRSSRRLAQQKI
NECVKSQSNRYGFCGNGTHIFSIVNSAPDGLLFLHTVLLPTDYKNVKAWSGICVDGI
YGYVLRQPNLVLYSDNGVFRVTSRVMFQPRLPVLSDFVQIYNCNVTFVNISRVELHT
VIPDYVDVNKTLQEFAQNLPKYVKPNFDLTPFNLTYLNLSSELKQLEAKLASLFQTT
VELQGLIDQINSTYVDLGSGEPEA
SEQ ID NO: 30-COR210547
MKLFLILLVLPLASCFFTCNSNANLSMLQLGVPDNSSTIVTGLLPTHWICANQSTSVY
SANGFFYIDVGNHRSAFALHTGYYDVNQYYIYVTNEIGLNASVTLKICKFGINTTFDF
LSNSSSSFDCIVNLLFTEQLGAPLGITISGETVRLHLYNVTRTFYVPAAYKLTKLSVKC
YFNYSCVFSVVNATVTVNVTTHNGRVVNYTVCDDCNGYTDNIFSVQQDGRIPNGFP
FNNWFLLTNDSTLVDGVSRLYQPLRLTCLWPVPGLKSSTGFVYFNATGSDVNCNGY
QHNSVADVMRYNLNFSANSVDNLKSGVIVFKTLQYDVLFYCSNSSSGVLDTTIPFGP
SSQPYYCFINSTINTTHVSTFVGVLPPTVREIVVARTGQFYINGFKYFDLGFIEAVNFN
VTTASATDFWTVAFATFVDVLVNVSATKIQNLLYCDSPFEKLQCEHLQFGLQDGFYS
ANFLDDNVLPETYVALPIYYQHTDINFTATASFGGSCYVCKPHQVNISLNGNTSVCV
RTSHFSIRYIYNRVKSGSPGDSSWHIYLKSGTCPFSFSKLNNFQKFKTICFSTVAVPGS
CNFPLEATWHYTSYTIVGALYVTWSEGNSITGVPYPVSGIREFSNLVLNNCTKYNIYD
YVGTGIIRSSNQSLAGGITYVSNSGNLLGFKNVSTGNIFIVTPCNQPDQVAVYQQSIIG
AMTAVNESRYGLQNLLQLPNFYYVSNGGNNCTTAVMTYSNFGICADGSLIPVRPRN
SSDNGISAIITANLSIPSNWTTSVQVEYLQITSTPIVVDCATYVCNGNPRCKNLLKQYT
SACKTIEDALRLSAHLETNDVSSMLTFDSNAFSLANVTSFGDYNLSSVLPQRNIHSSRI
AGRSALEDLLFSKVVTSGLGTVDVDYKSCTKGLSIADLACAQYYNGIMVLPGVADA
ERMAMYTGSLIGGMVLGGLTSAAAIPFSLALQARLNYVALQTDVLQENQKILAASF
NKAINNIVASFSSVNDPITQTAEAIHTVTIALNKIQDVVNQQGSALNHLTSQLRHINFQ
AISNSIQAIYDRLDPIQADQQVDRLITGRLAALNAFVSQVLNKYTEVRSSRRLAQQKI
NECVKSQSNRYGFCGNGTHIFSIVNSAPDGLLFLHTVLLPTDYKNVKAWSGICVDGI
YGYVLRQPNLVLYSDNGVFRVTSRVMFQPRLPVLSDFVQIYNCNVTFVNISRVELHT
VIPDYVDVNKTLQEFAQNLPKYVKPNFDLTPFNLTYLNLSSELKQLEAKIASLFQTTV
ELQGLIDQINSTYVDLGSGEPEA
SEQ ID NO: 31-COR210548
MKLFLILLVLPLASCFFTCNSNANLSMLQLGVPDNSSTIVTGLLPTHWICANQSTSVY
SANGFFYIDVGNHRSAFALHTGYYDVNQYYIYVTNEIGLNASVTLKICKFGINTTFDF
LSNSSSSFDCIVNLLFTEQLGAPLGITISGETVRLHLYNVTRTFYVPAAYKLTKLSVKC
YFNYSCVFSVVNATVTVNVTTHNGRVVNYTVCDDCNGYTDNIFSVQQDGRIPNGFP
FNNWFLLTNDSTLVDGVSRLYQPLRLTCLWPVPGLKSSTGFVYFNATGSDVNCNGY
QHNSVADVMRYNLNFSANSVDNLKSGVIVFKTLQYDVLFYCSNSSSGVLDTTIPFGP
SSQPYYCFINSTINTTHVSTFVGVLPPTVREIVVARTGQFYINGFKYFDLGFIEAVNFN
VTTASATDFWTVAFATFVDVLVNVSATKIQNLLYCDSPFEKLQCEHLQFGLQDGFYS
ANFLDDNVLPETYVALPIYYQHTDINFTATASFGGSCYVCKPHQVNISLNGNTSVCV
RTSHFSIRYIYNRVKSGSPGDSSWHIYLKSGTCPFSFSKLNNFQKFKTICFSTVAVPGS
CNFPLEATWHYTSYTIVGALYVTWSEGNSITGVPYPVSGIREFSNLVLNNCTKYNIYD
YVGTGIIRSSNQSLAGGITYVSNSGNLLGFKNVSTGNIFIVTPCNQPDQVAVYQQSIIG
AMTAVNESRYGLQNLLQLPNFYYVSNGGNNCTTAVMTYSNFGICADGSLIPVRPRN
SSDNGISAIITANLSIPSNWTTSVQVEYLQITSTPIVVDCATYVCNGNPRCKNLLKQYT
SACKTIEDALRLSAHLETNDVSSMLTFDSNAFSLANVTSFGDYNLSSVLPQRNIHSSRI
AGRSALEDLLFSKVVTSGLGTVDVDYKSCTKGLSIADLACAQYYNGIMVLPGVADA
ERMAMYTGSLIGGMVLGGLTSAAAIPFSLALQARLNYVALQTDVLQENQKILAASF
NKAINNIVASFSSVNDPITQTAEAIHTVTIALNKIQDVVNQQGSALNHLTSQLRHNFQ
AISNSIQAIYDRLDPIQADQQVDRLITGRLAALNAFVSQVLNKYTEVRSSRRLAQQKI
NECVKSQSNRYGFCGNGTHIFSIVNSAPDGLLFLHTVLLPTDYKNVKAWSGICVDGI
YGYVLRQPNLVLYSDNGVFRVTSRVMFQPRLPVLSDFVQIYNCNVTFVNISRVELHT
VIPDYVDVNKTLQEFAQNLPKYVKPNFDLTPFNLTYLNLSSELKQLEAKTASLFQTV
VELQGLIDQINSTYVDLGSGEPEA
SEQ ID NO: 32-COR210549
MKLFLILLVLPLASCFFTCNSNANLSMLQLGVPDNSSTIVTGLLPTHWICANQSTSVY
SANGFFYIDVGNHRSAFALHTGYYDVNQYYIYVTNEIGLNASVTLKICKFGINTTFDF
LSNSSSSFDCIVNLLFTEQLGAPLGITISGETVRLHLYNVTRTFYVPAAYKLTKLSVKC
YFNYSCVFSVVNATVTVNVTTHNGRVVNYTVCDDCNGYTDNIFSVQQDGRIPNGFP
FNNWFLLTNDSTLVDGVSRLYQPLRLTCLWPVPGLKSSTGFVYFNATGSDVNCNGY
QHNSVADVMRYNLNFSANSVDNLKSGVIVFKTLQYDVLFYCSNSSSGVLDTTIPFGP
SSQPYYCFINSTINTTHVSTFVGVLPPTVREIVVARTGQFYINGFKYFDLGFIEAVNFN
VTTASATDFWTVAFATFVDVLVNVSATKIQNLLYCDSPFEKLQCEHLQFGLQDGFYS
ANFLDDNVLPETYVALPIYYQHTDINFTATASFGGSCYVCKPHQVNISLNGNTSVCV
RTSHFSIRYIYNRVKSGSPGDSSWHIYLKSGTCPFSFSKLNNFQKFKTICFSTVAVPGS
CNFPLEATWHYTSYTIVGALYVTWSEGNSITGVPYPVSGIREFSNLVLNNCTKYNIYD
YVGTGIIRSSNQSLAGGITYVSNSGNLLGFKNVSTGNIFIVTPCNQPDQVAVYQQSIIG
AMTAVNESRYGLQNLLQLPNFYYVSNGGNNCTTAVMTYSNFGICADGSLIPVRPRN
SSDNGISAIITANLSIPSNWTTSVQVEYLQITSTPIVVDCATYVCNGNPRCKNLLKQYT
SACKTIEDALRLSAHLETNDVSSMLTFDSNAFSLANVTSFGDYNLSSVLPQRNIHSSRI
AGRSALEDLLFSKVVTSGLGTVDVDYKSCTKGLSIADLACAQYYNGIMVLPGVADA
ERMAMYTGSLIGGMVLGGLTSAAAIPFSLALQARLNYVALQTDVLQENQKILAASF
NKAINNIVASFSSVNDPITQTAEAIHTVTIALNKIQDVVNQQGSALNHLTSQLRHNFQ
AISNSIQAIYDRLDPIQADQQVDRLITGRLAALNAFVSQVLNKYTEVRSSRRLAQQKI
NECVKSQSNRYGFCGNGTHIFSIVNSAPDGLLFLHTVLLPTDYKNVKAWSGICVDGI
YGYVLRQPNLVLYSDNGVFRVTSRVMFQPRLPVLSDFVQIYNCNVTFVNISRVELHT
VIPDYVDVNKTLQEFAQNLPKYVKPNFDLTPFNLTYLNLSSELKQLEAKTASLFQTL
VELQGLIDQINSTYVDLGSGEPEA
SEQ ID NO: 33-COR210550
MKLFLILLVLPLASCFFTCNSNANLSMLQLGVPDNSSTIVTGLLPTHWICANQSTSVY
SANGFFYIDVGNHRSAFALHTGYYDVNQYYIYVTNEIGLNASVTLKICKFGINTTFDF
LSNSSSSFDCIVNLLFTEQLGAPLGITISGETVRLHLYNVTRTFYVPAAYKLTKLSVKC
YFNYSCVFSVVNATVTVNVTTHNGRVVNYTVCDDCNGYTDNIFSVQQDGRIPNGFP
FNNWFLLTNDSTLVDGVSRLYQPLRLTCLWPVPGLKSSTGFVYFNATGSDVNCNGY
QHNSVADVMRYNLNFSANSVDNLKSGVIVFKTLQYDVLFYCSNSSSGVLDTTIPFGP
SSQPYYCFINSTINTTHVSTFVGVLPPTVREIVVARTGQFYINGFKYFDLGFIEAVNFN
VTTASATDFWTVAFATFVDVLVNVSATKIQNLLYCDSPFEKLQCEHLQFGLQDGFYS
ANFLDDNVLPETYVALPIYYQHTDINFTATASFGGSCYVCKPHQVNISLNGNTSVCV
RTSHFSIRYIYNRVKSGSPGDSSWHIYLKSGTCPFSFSKLNNFQKFKTICFSTVAVPGS
CNFPLEATWHYTSYTIVGALYVTWSEGNSITGVPYPVSGIREFSNLVLNNCTKYNIYD
YVGTGIIRSSNQSLAGGITYVSNSGNLLGFKNVSTGNIFIVTPCNQPDQVAVYQQSIIG
AMTAVNESRYGLQNLLQLPNFYYVSNGGNNCTTAVMTYSNFGICADGSLIPVRPRN
SSDNGISAIITANLSIPSNWTTSVQVEYLQITSTPIVVDCATYVCNGNPRCKNLLKQYT
SACKTIEDALRLSAHLETNDVSSMLTFDSNAFSLANVTSFGDYNLSSVLPQRNIHSSRI
AGRSALEDLLFSKVVTSGLGTVDVDYKSCTKGLSIADLACAQYYNGIMVLPGVADA
ERMAMYTGSLIGGMVLGGLTSAAAIPFSLALQARLNYVALQTDVLQENQKILAASF
NKAINNIVASFSSVNDPITQTAEAIHTVTIALNKIQDVVNQQGSALNHLTSQLRHNFQ
AISNSIQAIYDRLDPIQADQQVDRLITGRLAALNAFVSQVLNKYTEVRSSRRLAQQKI
NECVKSQSNRYGFCGNGTHIFSIVNSAPDGLLFLHTVLLPTDYKNVKAWSGICVDGI
YGYVLRQPNLVLYSDNGVFRVTSRVMFQPRLPVLSDFVQIYNCNVTFVNISRVELHT
VIPDYVDVNKTLQEFAQNLPKYVKPNFDLTPFNLTYLNLSSELKQLEAKTASLFQTIV
ELQGLIDQINSTYVDLGSGEPEA
SEQ ID NO: 34-COR210551
MKLFLILLVLPLASCFFTCNSNANLSMLQLGVPDNSSTIVTGLLPTHWICANQSTSVY
SANGFFYIDVGNHRSAFALHTGYYDVNQYYIYVTNEIGLNASVTLKICKFGINTTFDF
LSNSSSSFDCIVNLLFTEQLGAPLGITISGETVRLHLYNVTRTFYVPAAYKLTKLSVKC
YFNYSCVFSVVNATVTVNVTTHNGRVVNYTVCDDCNGYTDNIFSVQQDGRIPNGFP
FNNWFLLTNDSTLVDGVSRLYQPLRLTCLWPVPGLKSSTGFVYFNATGSDVNCNGY
QHNSVADVMRYNLNFSANSVDNLKSGVIVFKTLQYDVLFYCSNSSSGVLDTTIPFGP
SSQPYYCFINSTINTTHVSTFVGVLPPTVREIVVARTGQFYINGFKYFDLGFIEAVNFN
VTTASATDFWTVAFATFVDVLVNVSATKIQNLLYCDSPFEKLQCEHLQFGLQDGFYS
ANFLDDNVLPETYVALPIYYQHTDINFTATASFGGSCYVCKPHQVNISLNGNTSVCV
RTSHFSIRYIYNRVKSGSPGDSSWHIYLKSGTCPFSFSKLNNFQKFKTICFSTVAVPGS
CNFPLEATWHYTSYTIVGALYVTWSEGNSITGVPYPVSGIREFSNLVLNNCTKYNIYD
YVGTGIIRSSNQSLAGGITYVSNSGNLLGFKNVSTGNIFIVTPCNQPDQVAVYQQSIIG
AMTAVNESRYGLQNLLQLPNFYYVSNGGNNCTTAVMTYSNFGICADGSLIPVRPRN
SSDNGISAIITANLSIPSNWTTSVQVEYLQITSTPIVVDCATYVCNGNPRCKNLLKQYT
SACKTIEDALRLSAHLETNDVSSMLTFDSNAFSLANVTSFGDYNLSSVLPQRNIHSSRI
AGRSALEDLLFSKVVTSGLGTVDVDYKSCTKGLSIADLACAQYYNGIMVLPGVADA
ERMAMYTGSLIGGMVLGGLTSAAAIPFSLALQARLNYVALQTDVLQENQKILAASF
NKAINNIVASFSSVNDPITQTAEAIHTVTIALNKIQDVVNQQGSALNHLTSQLRHNFQ
AISNSIQAIYDRLDPIQADQQVDRLITGRLAALNAFVSQVLNKYTEVRSSRRLAQQKI
NECVKSQSNRYGFCGNGTHIFSIVNSAPDGLLFLHTVLLPTDYKNVKAWSGICVDGI
YGYVLRQPNLVLYSDNGVFRVTSRVMFQPRLPVLSDFVQIYNCNVTFVNISRVELHT
VIPDYVDVNKTLQEFAQNLPKYVKPNFDLTPFNLTYLNLSSELKQLEAKVASLFQTV
VELQGLIDQINSTYVDLGSGEPEA
SEQ ID NO: 35-COR210560
MKLFLILLVLPLASCFFTCNSNANLSMLQLGVPDNSSTIVTGLLPTHWICANQSTSVY
SANGFFYIDVGNHRSAFALHTGYYDVNQYYIYVTNEIGLNASVTLKICKFGINTTFDF
LSNSSSSFDCIVNLLFTEQLGAPLGITISGETVRLHLYNVTRTFYVPAAYKLTKLSVKC
YFNYSCVFSVVNATVTVNVTTHNGRVVNYTVCDDCNGYTDNIFSVQQDGRIPNGFP
FNNWFLLTNDSTLVDGVSRLYQPLRLTCLWPVPGLKSSTGFVYFNATGSDVNCNGY
QHNSVADVMRYNLNFSANSVDNLKSGVIVFKTLQYDVLFYCSNSSSGVLDTTIPFGP
SSQPYYCFINSTINTTHVSTFVGVLPPTVREIVVARTGQFYINGFKYFDLGFIEAVNFN
VTTASATDFWTVAFATFVDVLVNVSATKIQNLLYCDSPFEKLQCEHLQFGLQDGFYS
ANFLDDNVLPETYVALPIYYQHTDINFTATASFGGSCYVCKPHQVNISLNGNTSVCV
RTSHFSIRYIYNRVKSGSPGDSSWHIYLKSGTCPFSFSKLNNFQKFKTICFSTVAVPGS
CNFPLEATWHYTSYTIVGALYVTWSEGNSITGVPYPVSGIREFSNLVLNNCTKYNIYD
YVGTGIIRSSNQSLAGGITYVSNSGNLLGFKNVSTGNIFIVTPCNQPDQVAVYQQSIIG
AMTAVNESRYGLQNLLQLPNFYYVSNGGNNCTTAVMTYSNFGICADGSLIPVRPRN
SSDNGISAIITANLSIPSNWTTSVQVEYLQITSTPIVVDCATYVCNGNPRCKNLLKQYT
SACKTIEDALRLSAHLETNDVSSMLTFDSNAFSLANVTSFGDYNLSSVLPQRNIHSSRI
AGRSALEDLLFSKVVTSGLGTVDVDYKSCTKGLSIADLACAQYYNGIMVLPGVADA
ERMAMYTGSLIGGMVLGGLTSAAAIPFSLALQARLNYVALQTDVLQENQKILAASF
NKAINNIVASFSSVNDPITQTAEAIHTVTIALNKIQDVVNQQGSALNHLTSQLRHNFQ
AISNSIQAIYDRLDPIQADQQVDRLITGRLAALNAFVSQVLNKYTEVRSSRRLAQQKI
NECVKSQSNRYGFCGNGTHIFSIVNSAPDGLLFLHTVLLPTDYKNVKAWSGICVDGI
YGYVLRQPNLVLYSDNGVFRVTSRVMFQPRLPVLSDFVQIYNCNVTFVNISRVELHT
VIPDYVDVNKTLQEFAQNLPKYVKPNFDLTPFNLTYLNLSSELKQLEAKTASLFQTT
VELQGLIDQINSTYVDLGSGEPEA
SEQ ID NO: 36-Sendai virus
MATYIQRVQCISALLSVVLTTLVSCQIPRDRLSNIGVIVDEGKSLKIAGSHESRYIVLSL
VPGIDLENGCGTAQVIQYKSLLNRLLIPLRDALDLQEALITVINDTMTGADVPQSRFF
GAVIGTIALGVATSAQITAGIALAEAREAKRDIALIKESMTKTHKSIELLQNAVGEQIL
ALKTLQDFVNDEIKPAISELGCETAALRLGIKLTQHYSELLTAFGSNFGTIGEKSLTLQ
ALSSLYSANITEIMTTIRTGQSNIYDVIYTEQIKGTVIDVDLERYMVTLSVKIPILSEVP
GVLIHKASSISYNIDGEEWYVTVPSHILSRASFLGGANIADCVESRLTYICPRDPAQLIP
DSQQKCILGDTTRCPVTKVVDNIIPKFAFVNGGVVANCIASTCTCGTGRRPISQDRSK
GVVFLTHDNCGLIGVNGIELYANRKGHDATWGVQNLTVGPAIAIRPVDISLNLAAAT
DFLQDSRAELEKARKILSEVGRWYNSGATLITIIVVMIVVLVVIIVIVIVLYRLRRSML
MSNPAGRISRDTYTLEPKIRHMYTNGGFDAMTEKR
SEQ ID NO: 37-PIV2
MHHLHPMIVCIFVMYTGIVGSDAIAGDQLLNIGVIQSKIRSLMYYTDGGASFIVVKLL
PNLPPSNGTCNITSLDAYNVTLFKLLTPLIENLSKISAVTDTKTRQERFAGVVIGLAAL
GVATAAQITAAVAIVKANANAAAINNLASSIQSTNKAVSDVIDASRTIATAVQAIQD
HINGAIVNGITSASCRAHDALIGSILNLYLTELTTIFHNQITNPALTPLSIQALRILLGST
LPIVIESKLNTNLNTAELLSSGLLTGQIISISPMYMQMLIQINVPTFIMQPGAKVIDLIAI
SANHKLQEVVVQVPNRILEYANELQNYPANDCVVTPNSVFCRYNEGSPIPESQYQCL
RGNLNSCTFTPIIGNFLKRFAFANGVLYANCKSLLCKCADPPHVVSQDDTQGISIIDIK
RCSEMMLDTFSFRITSTFNATYVTDFSMINANIVHLSPLDLSNQINSINKSLKSAEDWI
ADSNFFANQARTAKTLYSLSAIALILSVITLVVVGLLIAYIIKLVSQIHQFRALAATTM
FHRENPAFFSKNNHGNIYGIS
SEQ ID NO: 38-PIV4
MGVKGLSLVMIGLLISPITNLDITHLMNLGTVPTAIRSLVYYTYSKPSYLTVDLIPNLK
NLDPKCNYSSLNYYNKTALSLIQPIADNINRLTKPITSSEIQSRFFGAVIGTVALGVAT
AAQVTAAIGLAKAQENARLILTLKKAAKETNDAVRDLIKSNKIVARMISAIQNQINTII
QPAIDRINCQIKDLQVANILNLYLTEITTVFHNQLTNPALESISIQALKSLLGSTLPEVL
SKLDLNNISAASVMASGLIKGQIIAVDIPTMTLVLMVQIPSISPLRQAKIIDLTSITIHTN
NQEVQAVVPDRVLEIGSEILGFDGSVCQITKDTVFCPYNDAYILPIQQKRCLQGQTRD
CVFTPVAGTFPRRFLTTYGTIVANCRNLVCSCLRPPQIIYQPDENPVTIIDKDLCTTLTL
DSITIEIQKSINSTFRREVVLESTQVRSLTPLDLSTDLSQYNQLLKSAEDHIQRSNDYLN
SINPSIVNNSAIIILIILCILLILTVTICIIWLKYLTKEVKNVARNQRLNRDADLIYKIPSQI
PLPR
SEQ ID NO: 39-PIV5
MGTIIQFLVVSCLLAGAGSLDPAALMQIGVIPTNVRQLMYYTEASSAFIVVKLMPTID
SPISGCNITSISSYNATVTKLLQPIGENLETIRNQLIPTRRRRRFAGVVIGLAALGVATA
AQVTAAVALVKANENAAAILNLKNAIQKTNAAVADVVQATQSLGTAVQAVQDHIN
SVVSPAITAANCKAQDAIIGSILNLYLTELTTIFHNQITNPALSPITIQALRILLGSTLPTV
VEKSFNTQISVAELLSSGLLTGQIVGLDLTYMQMVIKIELPTLAVQPATQIIDLATISAF
INNQEVMAQLPTRVIVTGSLIQAYPASQCTITPNTVYCRYNDARVLSDDTMACLQGN
LTRCTFSPVVGSFLTRFVLFDGIVYANCRSMLCKCMQPAAVILQPSSSPVTVIDMYKC
VSLRLDNLRFTITQLANVTYNSTIKLETSQILPIAPLDISQNLAAVNKSLSDALQHLAQ
SDTYLSAITSATTTSVLSIIAICLGSLGLILIILLSVVVWKLLTIVAANRNRMENFVYHN
SAFHHSRSDLSEKNQPATLGTR
SEQ ID NO: 40-Mumps virus
MKAFLVTCLGFAVFSFSICVNINILQQIGYIKQQVRQLSYYSQSSSSYIVVKLLPNIQPT
DNSCEFKSVTQYNKTLSNLLLPIAENINNIASPSPGSRRHKRFAGIAIGIAALGVATAA
QVTAAVSLVQAQTNARAIAAMKNSIQATNRAVFEVKEGTQQLAIAVQAIQDHINTIM
NTQLNNMSCQILDNQLATSLGLYLTELTTVFQPQLINPALSPISIQALRSLLGSMTPAV
VQATLSTSISAAEILSAGLMEGQIVSVLLDEMQMIVKINIPTIVTQSNALVIDFYSISSFI
NNQESIIQLPDRILEIGNEQWSYPAKNCKLTRHNIFCQYNEAERLSLESKLCLAGNISA
CVFSPIAGSYMRRFVALDGTIVANCRSLTCLCKSPSYPIYQPDHHAVTTIDLTACQTLS
LDGLDFSIVSLSNITYAENLTISLSQTINTQPIDISTELSKVNASLQNAVKYIKESNHQL
QSVSVNSKIGAIIIAALVLSILSIIISLLFCCWAYIATKEIRRINFKTNHINTISSSVDDLIR
Y
SEQ ID NO: 41-Measles virus
MGLKVNVSAIFMAVLLTLQTPTGQIHWGNLSKIGVVGIGSASYKVMTRSSHQSLVIK
LMPNITLLNNCTRVEIAEYRRLLRTVLEPIRDALNAMTQNIRPVQSVASSRRHKRFAG
VVLAGAALGVATAAQITAGIALHQSMLNSQAIDNLRASLETTNQAIEAIRQAGQEMI
LAVQGVQDYINNELIPSMNQLSCDLIGQKLGLKLLRYYTEILSLFGPSLRDPISAEISIQ
ALSYALGGDINKVLEKLGYSGGDLLGILESRGIKARITHVDTESYFIVLSIAYPTLSEIK
GVIVHRLEGVSYNIGSQEWYTTVPKYVATQGYLISNFDESSCTFMPEGTVCSQNALY
PMSPLLQECLRGSTKSCARTLVSGSFGNRFILSQGNLIANCASILCKCYTTGTIINQDP
DKILTYIAADHCPVVEVNGVTIQVGSRRYPDAVYLHRIDLGPPISLERLDVGTNLGNA
IAKLEDAKELLESSDQILRSMKGLSSTSIVYILIAVCLGGLIGIPALICCCRGRCNKKGE
QVGMSRPGLKPDLTGTSKSYVRSL
SEQ ID NO: 42-Hendra virus
MATQEVRLKCLLCGIIVLVLSLEGLGILHYEKLSKIGLVKGITRKYKIKSNPLTKDIVIK
MIPNVSNVSKCTGTVMENYKSRLTGILSPIKGAIELYNNNTHDLVGDVKLAGVVMA
GIAIGIATAAQITAGVALYEAMKNADNINKLKSSIESTNEAVVKLQETAEKTVYVLTA
LQDYINTNLVPTIDQISCKQTELALDLALSKYLSDLLFVFGPNLQDPVSNSMTIQAISQ
AFGGNYETLLRTLGYATEDFDDLLESDSIAGQIVYVDLSSYYIIVRVYFPILTEIQQAY
VQELLPVSFNNDNSEWISIVPNFVLIRNTLISNIEVKYCLITKKSVICNQDYATPMTAS
VRECLTGSTDKCPRELVVSSHVPRFALSGGVLFANCISVTCQCQTTGRAISQSGEQTL
LMIDNTTCTTVVLGNIIISLGKYLGSINYNSESIAVGPPVYTDKVDISSQISSMNQSLQQ
SKDYIKEAQKILDTVNPSLISMLSMIILYVLSIAALCIGLITFISFVIVEKKRGNYSRLDD
RQVRPVSNGDLYYIGT
SEQ ID NO: 43-Newcastle disease virus
MGSRPFTKNPAPMMLTIRVALVLSCICPANSIDGRPFAAAGIVVTGDKAVNIYTSSQT
GSIIVKLLPNLPKDKEACAKAPLDAYNRTLTTLLTPLGDSIRRIQESVTTSGGGRQGRL
IGAIIGGVALGVATAAQITAAAALIQAKQNAANILRLKESIAATNEAVHEVTDGLSQL
AVAVGKMQQFVNDQFNKTAQELDCIKIAQQVGVELNLYLTELTTVFGPQITSPALNK
LTIQALYNLAGGNMDYLLTKLGIGNNQLSSLIGSGLITGNPILYDSQTQLLGIQVTLPS
VGNLNNMRATYLETLSVSTTRGFASALVPKVVTQVGSVIEELDTSYCIETDLDLYCT
RIVTFPMSPGIYSCLSGNTSACMYSKTEGALTTPYMTIKGSVIANCKMTTCRCVNPPG
IISQNYGEAVSLIDKQSCNVLSLGGITLRLSGEFDVTYQKNISIQDSQVIITGNLDISTEL
GNVNNSISNALNKLEESNRKLDKVNVKLTSTSALITYIVLTIISLVFGILSLILACYLMY
KQKAQQKTLLWLGNNTLDQMRATTKM
SEQ ID NO: 44-Avian orthoavulavirus 1
MGSKPSTRILAPLMLITRIMLTLSCIRLTSSLDGRPLAAAGIVVTGDKAVNVYTSSQTG
SIIVKLLPNMPRDKEACARAPLEAYNRTLTTLLTPLGDSIRKIQGSVSTSGGRRQKRFI
GAVIGSVALGVATAAQITAAAALIQAKQNAANILRLKESIAATNEAVHEVTDGLSQL
SVAVGKMQQFVNDQFNNTARELDCIKITQQVGVELNLYLTELTTVFGPQITSPALTQ
LTIQALYNLAGGNMDYLLTKLGIGNNQLSSLIGSGLITGYPILYDSHTQLLGIQVNLPS
VGNLNNMRATYLETLSVSTTKGYASALVPKVVTQVGSVIEELDTSYCIESDLDLYCT
RIVTFPMSPGIYSCLSGNTSACMYSKTEGALTTPYMALRGSVIANCKITTCRCTDPPGI
ISQNYGEAVSLIDRHSCNVLSLDGITLRLSGEFDATYQKNISILDSQVIVTGNLDISTEL
GNVNNSISNALDRLAESSSKLERVNVRLTSTSALITYIVLTVISLIFGALSLALACYLM
YKQKAQQKTLLWLGNNTLDQMRATTRA
SEQ ID NO: 45-Canine distemper virus
MHNKIPKKSKPLPHTRQDPLQQQSTKSAETKTSQGRYSITSAQRSTYHGPRTSDRSVH
YIMNRTRSCKQTSHRSDNIPPHRDHEGIIHHTPESVTQGASSWFKRRQSNATNAGSQC
TWLVLWCIGIASLFLCSKAQIHWNNLSTIGIIGTDSVHYKIMTRPSHQYLVIKLMPNV
SLIDNCTKAELGEYEKLLNSVLEPINQALTLMTNNVKPLQSVGSGRRQRRFAGVVLA
GAALGVATAAQITAGIALHQSNLNAQAIQSLRTSLEQSNKAIEEIREATQETVIAVQG
VQDYVNNELVPAMQHMSCELVGQRLGLKLLRYYTELLSIFGPSLRDPISAEISIQALS
YALGGEIHKILEKLGYSGNDMIAILESRGIKTKITHVDLPGKLIILSISYPTLSEVKGVIV
HRLEAVSYNIGSQEWYTTVPKYVATNGYLISNFDESSCVFVSESAICSQNSLYPMSPIL
QQCIRGDTSSCARTWVSGTMGNKFILSKGNIVANCASILCKCYSTSTIINQSPDKLLTF
IASDTCPLVEIDGVTIQVGGRQYPDMVYESKVALGPAISLERLDVGTNLGNALKKLD
DAKVLIDSSNQILETVKRSSFNFSSLLSVPILICTALALLLLIYCCKRRYRQTFKHNTKV
DPTFKPDLTGTSKSYVRSL
SEQ ID NO: 46-Feline morbillivirus
MNRIKVIIISSLLLSDITIAQIGWDNLTSIGVISTKQYDYKITTLNTNQLMVIKMVPNISS
IINCTKLELTKYRELVSGIIRPINESLELMNSYINMRAGSERFIGAVIAGVALGVATAA
QITSGIALHNSIMNKKQIQELRKALSATNKAIDEIRIAGERTLIAVQGVQDYINNIIIPM
QDKLQCDILSSQLSVALLRYYTNILTEFGPSIRDPVTSTISVQALSQAFNGNLQALLDG
LGYSGRDLRDLLESKSITGQIIHADMTDLFLVLRINYPSITEMQGVTIYGLNSITYHIGP
EEWYTIMPDFVAVQGFLISNFDERKCSITKSSILCQQNSIYPMSTEMQRCIKGEIRFCP
RSKAIGTLVNRFILTEGNLMANCLGIICRCYTSGQVITQDPSKLITIISQEECKEVGVDG
IRIMVGPRKLPDITFNARLEIGVPISLSKLDVGTDLAIASAKLNNSKALLEQSDKILYS
MSKLDSINSRIIGLILAIMIIFVIIITIIWIIYKKCRNKDNKFSTSIEPLYIPPSYNSPHSVVK
SI
SEQ ID NO: 47-Porcine respirovirus 1
MQVTTLGLAITLSIASLVTSQVPRDKLANLGIIIKDSKALKIAGSYENRYIVLSLVPTID
NVNGCGSIQIAKYKEMLERLLIPIKDALDLQESLIVIDNETTANTYHPQYRFVGAIIGTI
ALGVATAAQVTAGVALMEAREAKRDISMLKEAMEKTQNSIEKLQNSAGEQILALK
MLQDYVNGEIKPAIEELGCETAALKLGIALTQHYTELTNAFGSNLGSIGEKSLTLQAL
SSLYKTNITDILTATNLRKTDIYDIIYAEQVKGRVIDVDLRRYMVTISIKIPILSEIPGVLI
YEVSSISYNIDGAEWYAAVPDHILSKSAYIGGADVSDCIESGLTYICPRDPAQIIADNQ
QKCFLGHLDKCPVTKVIDNLVSKFAFINGGVVANCIASTCTCGEERIQVSQDRDKGV
TFLTHNNCGLIGVNGVELHANKKGSDATWNVSPIGVGPAVSLRPVDISLQIVAATNF
LNSSRKDLMKAKEVLNQVGNLKDLTVITIINIKIIIVLLICVIGLGILYHQLRSALEMRD
KMSALNNSSYSLEPRTVQVQVMKPISFTR
SEQ ID NO: 48-Mojiang virus
MALNKNMFSSLFLGYLLVYATTVQSSIHYDSLSKVGVIKGLTYNYKIKGSPSTKLMV
VKLIPNIDSVKNCTQKQYDEYKNLVRKALEPVKMAIDTMLNNVKSGNNKYRFAGAI
MAGVALGVATAATVTAGIALHRSNENAQAIANMKSAIQNTNEAVKQLQLANKQTL
AVIDTIRGEINNNIIPVINQLSCDTIGLSVGIRLTQYYSEIITAFGPALQNPVNTRITIQAIS
SVFNGNFDELLKIMGYTSGDLYEILHSELIRGNIIDVDVDAGYIALEIEFPNLTLVPNA
VVQELMPISYNIDGDEWVTLVPRFVLTRTTLLSNIDTSRCTITDSSVICDNDYALPMS
HELIGCLQGDTSKCAREKVVSSYVPKFALSDGLVYANCLNTICRCMDTDTPISQSLG
ATVSLLDNKRCSVYQVGDVLISVGSYLGDGEYNADNVELGPPIVIDKIDIGNQLAGIN
QTLQEAEDYIEKSEEFLKGVNPSIITLGSMVVLYIFMILIAIVSVIALVLSIKLTVKGNV
VRQQFTYTQHVPSMENINYVSH
SEQ ID NO: 49-Salmon aquaparamyxovirus
MAIKLIVRVAIILVSLSITFGQVDYGRLRKIGVFEKQTMNLKLSISASQRYMVIKTVPN
LGTVSSCGDKEMAAYKESIRKLISPMHDMIEYIKGEVVVEAATSVALNGTQVRFFGL
VVAIGALGLATSAQITAGIALHNSLENAKAIKGLSDAMKESNQAIQKLQDATAGTVI
ALNALQDQINTQIVPALNTLGCSVVSNTLGVALTRYYSELVQLFGPSLANPVEAPLSI
QAISGAFNGDLKGMIRDYGYSPSDLEDIIRTGAITGRVIDVDMEDLTIILEISLPTLLVM
RDTKVVNFGRITYNLNGSEWQTLSPDWIAIRNTLMSGVDLSTCVMSRQNLICKQDPT
FAIDHTVSQCLRGEITSCPRGRVVNSIAPRFAIVNGNVLGNCVATTCLCGDPGTPVIQ
DASSSLTIMSIDKCELVSIDGYNFRPGPPVVNTTFHLSIDDIGPEVSVNPIDISGALGKIE
QDLQSSKEHLANSDTILASINPKIINTNAAIGLIVVSSLGLIAAVLALCWLCCLTKSMV
GRDSTAFIGGKGPDMGPIMSSLEGMSF
SEQ ID NO: 50-Cetacean morbillivirus
MASNDNSVIYHSFLTVILLVVVTEGQIHWGNLSKIGIVGTGSASYKVMTRPSHQYLVI
KLMPNVTMIDNCTRTEVAEYRKLLKTVLEPVRNALTVITKNIKPIQSLTTSRRSKRFA
GVVLAGVALGVATAAQITAGIALHQSIMNSQSIDNLRTSLEKSNQAIEEIRQASQETV
LAVQGVQDFINNELIPSMHQLSCEMLGQKLGLKLLRYYTEILSIFGPSLRDPVSAEISI
QALTYALGGDINKVLEKLGYSGADLLAILESRGIKAKITHVDLEGYFIVLSIAYPTLSE
VKGVIVHRLEAVSYNLGSQEWYTTLPRYVATNGYLISNFDESSCAFMSEVTICSQNA
LYPMSPLLQQCLRGSTASCARSLVSGTIGNRFILSKGNLIANCASVLCKCYSTGTIISQ
DPDKLLTFVAADKCPLVEVDGITIQVGSREYPDSVYVSRIDLGPPISLEKLDVGTNLG
NALTKLNNAKELLDSSNQILENVRRGSFGGAMYIGILVCAGALVILCVLVYCCKRYC
CSRVQIVPKSTPGLKPDLTGTTKSYVRSL
SEQ ID NO: 51-Reptilian ferlavirus
MKVTTLLIILILITSTLCQISFDNLEQVGVMFDKPKFLKITGPASTATMIIKLIPTLGTME
SCGTSAVNEYKKTLDTILIPLRDTINKLSTDITVLEGTPKTLSRREKRFVGIAIAVGAV
ALATSAQITAGIALANTIKNAEAIESLKSSIQASNKAIQKVIDAQGRTVTVINGIQDHIN
SVINPALNQLGCDVAKNTLAISLTQYFSKLSLLFGPNLRNPVEQPLSVQAIAGLMDGD
INAVVSQLGYTPSDLLDLLSTESIVGTVTAVDMVNYMIQIEMSFPQYITIADTKVLEG
HRITFNDRGSEWQTQVPSTIAVRDILIAGVDPDGCSVTSTSYICKNDPTYAMSEVLTN
CFRGNTQECPRARITSTFATRFAIARSTVIANCVAAVCLCGDPGTPVVQKAEVTLTA
MTLDQCSLITVDGLQIKPSKSLANVTANFGNITLGPVVSVGDLDLSAELTKVQSDLKE
AQDRLDESNSILQGINNKILTAPTSIALIVVSVIIVLIIIGIISWLAWLTRAVKRSSIRSER
VTPNTYNNLGFIK
SEQ ID NO: 52 Langya henipavirus
MAFLKSAIICYLLFYPHIVKSSLHYDSLSKVGIIKGLTYNYKIKGSPSTKLMVVKLIPNI
DGVRNCTQKQFDEYKNLVKNVLEPVKLALNAMLDNVKSGNNKYRFAGAIMAGVA
LGVATAATVTAGIALHRSNENAQAIANMKNAIQNTNEAVKQLQLANKQTLAVIDTI
RGEINNNIIPVINQLSCDTIGLSVGIKLTQYYSEILTAFGPALQNPVNTRITIQAISSVEN
RNFDELLKIMGYTSGDLYEILHSGLIRGNIIDVDVEAGYIALEIEFPNLTLVPNAVVQE
LMPISYNVDGDEWVTLVPRFVLTRTTLLSNIDTSRCTVTESSVICDNDYALPMSYELI
GCLQGDTSKCAREKVVSSYVPRFALSDGLVYANCLNTICRCMDTDTPISQSLGTTVS
LLDNKKCLVYQVGDILISVGSYLGEGEYSADNVELGPPVVIDKIDIGNQLAGINQTLQ
NAEDYIEKSEEFLKGINPSIITLGSMAVLYIFMIVIAVISIIALVLSIKLTVKGNVVRQQF
AYTQHVPSMENVNYVSH
SEQ ID NO: 53 NL63
MKLFLILLVLPLASCFFTCNSNANLSMLQLGVPDNSSTIVTGLLPTHWICANQSTSVY
SANGFFYIDVGNHRSAFALHTGYYDVNQYYIYVTNEIGLNASVTLKICKFGINTTFDF
LSNSSSSFDCIVNLLFTEQLGAPLGITISGETVRLHLYNVTRTFYVPAAYKLTKLSVKC
YFNYSCVFSVVNATVTVNVTTHNGRVVNYTVCDDCNGYTDNIFSVQQDGRIPNGFP
FNNWFLLTNDSTLVDGVSRLYQPLRLTCLWPVPGLKSSTGFVYFNATGSDVNCNGY
QHNSVADVMRYNLNFSANSVDNLKSGVIVFKTLQYDVLFYCSNSSSGVLDTTIPFGP
SSQPYYCFINSTINTTHVSTFVGVLPPTVREIVVARTGQFYINGFKYFDLGFIEAVNFN
VTTASATDFWTVAFATFVDVLVNVSATKIQNLLYCDSPFEKLQCEHLQFGLQDGFYS
ANFLDDNVLPETYVALPIYYQHTDINFTATASFGGSCYVCKPHQVNISLNGNTSVCV
RTSHFSIRYIYNRVKSGSPGDSSWHIYLKSGTCPFSFSKLNNFQKFKTICFSTVAVPGS
CNFPLEATWHYTSYTIVGALYVTWSEGNSITGVPYPVSGIREFSNLVLNNCTKYNIYD
YVGTGIIRSSNQSLAGGITYVSNSGNLLGFKNVSTGNIFIVTPCNQPDQVAVYQQSIIG
AMTAVNESRYGLQNLLQLPNFYYVSNGGNNCTTAVMTYSNFGICADGSLIPVRPRN
SSDNGISAIITANLSIPSNWTTSVQVEYLQITSTPIVVDCATYVCNGNPRCKNLLKQYT
SACKTIEDALRLSAHLETNDVSSMLTFDSNAFSLANVTSFGDYNLSSVLPQRNIHSSRI
AGRSALEDLLFSKVVTSGLGTVDVDYKSCTKGLSIADLACAQYYNGIMVLPGVADA
ERMAMYTGSLIGGMVLGGLTSAAAIPFSLALQARLNYVALQTDVLQENQKILAASF
NKAINNIVASFSSVNDAITQTAEAIHTVTIALNKIQDVVNQQGSALNHLTSQLRHNFQ
AISNSIQAIYDRLDSIQADQQVDRLITGRLAALNAFVSQVLNKYTEVRSSRRLAQQKI
NECVKSQSNRYGFCGNGTHIFSIVNSAPDGLLFLHTVLLPTDYKNVKAWSGICVDGI
YGYVLRQPNLVLYSDNGVFRVTSRVMFQPRLPVLSDFVQIYNCNVTFVNISRVELHT
VIPDYVDVNKTLQEFAQNLPKYVKPNFDLTPFNLTYLNLSSELKQLEAKTASLFQTT
VELQGLIDQINSTYVDLKLLNRFENYIKWPWWVWLIISVVFVVLLSLLVFCCLSTGCC
GCCNCLTSSMRGCCDCGSTKLPYYEFEKVHVQ
SEQ ID NO: 54-229E
MFVLLVAYALLHIAGCQTTNGTNTSHSVCNGCVGHSENVFAVESGGYIPSNFAFNN
WFLLTNTSSVVDGVVRSFQPLLLNCLWSVSGSRFTTGFVYFNGTGRGDCKGFYSNA
SSDVIRYNINFEENLRRGTILFKTSYGAVVFYCTNNTLVSGDAHIPSGTVLGNFYCFV
NTTIDNETTSAFVGALPKTVREFVISRTGHFYINGYRYFSLGNVEAVNFNVTNAATTV
CTVALASYADVLVNVSQTAIANIIYCNSVINRLRCDQLSFDVPDGFYSTSPIQSVELPV
SIVSLPVYHKHTFIVLYVNFELRRGPGRCYNCRPAVVNITLANFNETKGPLCVDTSHF
TTQFVGNVKLDRWSASINTGNCPFSFGKVNNFVKFGSVCFSLKDIPGGCAMPIMANL
VNHKSHNIGSLYVSWSDGDVITGVPKPVEGVSSFMNVTLNKCTKYNIYDVSGVGVIR
ISNDTFLNGITYTSTSGNLLGFKDVTNGTIYSITPCNPPDQLVVYQQAVVGAMLSENF
TSYGFSNVVEMPKFFYASNGTYNCTDAVLTYSSFGVCADGSIIAVQPRNVSYDSVSAI
VTANLSIPSNWTTSVQVEYLQITSTPIVVDCSTYVCNGNVRCVELLKQYTSACKTIED
ALRNSAMLESADVSEMLTFDKKAFTLANVSSFGDYNLSSVIPSLPRSGSRVAGRSAIE
DILFSKLVTSGLGTVDADYKKCTKGLSIADLACAQYYNGIMVLPGVADAERMAMYT
GSLIGGIALGGLTSAASIPFSLAIQSRLNYVALQTDVLQENQKILAASENKAMTNIVDA
FTGVNDAITQTSQALQTVATALNKIQDVVNQQGNSLNHLTSQLRQNFQAISSSIQAIY
DRLDIIQADQQVDRLITGRLAALNVFVSHTLTKYTEVRASRQLAQQKVNECVKSQSK
RYGFCGNGTHIFSLVNAAPEGLVFLHTVLLPTQYKDVEAWSGLCVDGINGYVLRQP
NLALYKEGNYYRITSRIMFEPRIPTIADFVQIENCNVTFVNISRSELQTIVPEYIDVNKT
LQELSYKLPNYTVPDLVVEQYNQTILNLTSEISTLENKSAELNYTVQKLQTLIDNINST
LVDLKWLNRVETYIKWPWWVWLCISVVLIFVVSMLLLCCCSTGCCGFFSCFASSIRG
CCESTKLPYYDVEKIHIQ
SEQ ID NO: 55-Feline Infectious Peritonitis Virus (FIPV)
MIFIILTLLSVAKSEDAPHGVTLPQFNTSHNNERFELNFYNFLQTWDIPPNTETILGGY
LPYCGAGVNCGWYNFSQSVGQNGKYAYINTQNLNIPNVHGVYFDVREHNNDGEW
DDRDKVGLLIAIHGNSKYSLLMVLQDAVEANQPHVAVKICHWKPGNISSYHAFSVN
LGDGGQCVFNQRFSLDTVLTTNDFYGFQWTDTYVDIYLGGTITKVWVDNDWSIVEA
SISYHWNRINYGYYMQFVNRTTYYAYNNTGGANYTQLQLSECHTDYCAGYAKNVF
VPIDGKIPEDFSFSNWFLLSDKSTLVQGRVLSSQPVFVQCLRPVPSWSNNTAVVHFKN
DAFCPNVTADVLRFNLNFSDTDVYTDSTNDEQLFFTFEDNTTASIACYSSANVTDFQP
ANNSVSHIPFGKTAHFCFANFSHSIVSRQFLGILPPTVREFAFGRDGSIFVNGYKYFSLP
AIRSVNFSISSVEEYGFWTIAYTNYTDVMVDVNGTAITRLFYCDSPLNRIKCQQLKHE
LPDGFYSASMLVKKDLPKTFVTMPQFYHWMNVTLHVVLNDTEKKYDIILAKAPELA
ALADVHFEIAQANGSVTNVTSLCVQARQLALFYKYTSLQGLYTYSNLVELQNYDCP
FSPQQFNNYLQFETLCFDVNPAVAGCKWSLVHDVQWRTQFATITVSYKHGSMITTH
AKGHSWGFQDTSVLVKDECTDYNIYGFQGTGIIRNTTSRLVAGLYYTSISGDLLAFK
NSTTGEIFTVVPCDLTAQVAVINDEIVGAITAVNQTDLFEFVNNTQARRSRSSTPNFV
TSYTMPQFYYITKWNNDTSSNCTSAITYSSFAICNTGEIKYVNVTHVEIVDDSIGVIKP
VSTGNISIPKNFTVAVQAEYIQIQVKPVVVDCATYVCNGNTHCLKLLTQYTSACQTIE
NALNLGARLESLMLNDMITVSDRGLELATVERFNATALGGEKLGGLYFDGLSSLLPP
KIGKRSAVEDLLFNKVVTSGLGTVDDDYKKCSSGTDVADLVCAQYYNGIMVLPGV
VDGNKMSMYTASLIGGMALGSITSAVAVPFAMQVQARLNYVALQTDVLQENQKIL
ANAFNNAIGNITLALGKVSNAITTTSDGENSMASALTKIQSVVNQQGEALSQLTSQLQ
KNFQAISSSIAEIYNRLEKVEADAQVDRLITGRLAALNAYVSQTLTQYAEVKASRQIA
LEKVNECVKSQSNRYGFCGNGTHLFSLVNSAPEGLLFFHTVLLPTEWEEVTAWSGIC
VNDTYAYVLKDFDHSIFSYNGTYMVTPRNMFQPRKPQMSDFVQITSCEVTFLNMTY
TTFQEIVIDYIDINKTIADMLEQYNPNYTTPELNLLLDIFNQTKLNLTAEIDQLEQRAD
NLTTIAHELQQYIDNLNKTLVDLDWLNRIETYVKWPWYVWLLIGLVVVFCIPLLLFC
CLSTGFCGCFGCVGSCCHSLCSRRQFETYEPIEKVHIH
SEQ ID NO: 56-Porcine Epidemic Diarrhea Virus (PEDV)
MKSLNYFWLFLPVLSTFSLPQDVTRCSANTNFRRFFSKFNVQAPAVVVLGGYLPTGE
NHGVNSTWYCAGQHPTASGVHGVFLSHIRGGHGFEIGISQEPFDPSGYQLYLHKATN
GNTNATARLRICQFPNIKRLGPTANNDVTTGRNCLFNKAIPAHMSEHSVVGVTWDN
DRVTVFSDKIYHFYFKNDWSRVATKCYNSGGCAMQYVYEPTYYMLNVTSAGEDGI
SYQPCTANCIGYAANVFATEPNGHIPEGFSFNNWFLLSNDSTLLHGKVVSNQPLLVN
CLLAIPKIYGLGQFFSFNQTMDGVCNGAAAQRAPEALRFNINDTSAILAEGSIVLHTA
LGTNLSFVCSNSSDTHLATFAIPLGATQVPYYCFLKVDTYNSTVYKFLAVLPPTVREI
VITKYGDVYVNGFGYLHLGLLDAVTINFTGHGTDDDVSGFWTIASTNFVDALIEVQG
TAIQRILYCDDPVSQLKCSQVAFDLDDGFYPISSRNLLSHEQPISFVTPPSFNDHSFVNI
TVSASFGGRSGANLIASDTTINGFSSFCVDTRQFTISLFYNVTNSYGYVSKSQDSNCPF
TLQSVNDYLSFSKFCVSTSLLASACTIDLFGYPEFGSGVKFTSLYFQFTKGELITGTPK
PLQGVTDVSFMTLDVCTKYTIYGFKGEGIITLTNSSFLAGVYYTSDSGQLLAFKNVTS
GAVYSVTPCSFSEQAVYVDDDIVGVISSLSNSTFNNTRELPGFFYHSNDGSNCTEPVL
VYSNIGVCKSGSIGYVPLQDGQVKIAPMVNGNISIPTNFSMSIRTEYLQLYNTPVSVD
CVTYVCNGNSRCKQLLTQYTSACKTIESALQLSARLESVEVNSMLTISEEALQLATIS
SFNGDGYNFTNVLGVSVYDPASGRVVQKGSFIEDLLFNKVVINGLGTVDEDYKRCS
NGRSVADLVCAQYYSGVTVLPGVVDAEKLHMYSASLLGGMALGGLTTAAALPFSN
AVQARLNYLALQTDVLQRNQQLLAESFNSAIGNITSAFESVKEAISQTSNGLNTVAH
ALTKVQEVVNSQGSALTQLTIQLQHNFQAISSSIDDIYSRLDILSADVQVDRLITGRLS
ALNAFVAQTLTKYTEVQASRKLAQQKVNECVKSQSQRYGFCGGDGEHIFSLVQAAP
QGLLFLHTVLVPGDFVNVIAIDGLCVNGDIALTLREPGLVLFTHELQTYTATEYFVSS
RRMFEPRKPTVSDFVQIESCVVTYVNLTSDQLPDVIPDYIDVNKTLDEIPASLPNRIGP
SLPLDVFNATYLNLTGEIADLEQRSESLRNTTEELRSLIYNINNTLVDLEWLNRVETYI
KWPWWVWLIIFIVLIFVVSLLVFCCISTGCCGCCGCCCACFSGCCRGPRLQPYEVFEK
VHVQ
SEQ ID NO: 57-Transmissible Gastroenteritis Virus (TGEV)
MKKLFVVLVVMPLIYGDNFPCSKLTNRTIGNHWNLIETFLLNYSSRLSPNSDVVLGD
YFPTVQPWFNCIRNNSNDLYVTLENLKALYWDYATENITLNHKQRLNVVVNGYPYS
ITVTTTRNFNSAEGAIICICKGSPPTTTTESSLTCNWGSECRLNHKFPICPSNSEANCGN
MLYGLQWFADAVVAYLHGASYRISFENQWSGTVTLGDMRATTLETAGTLVDLWW
FNPVYDVSYYRVNNKNGTTVVSNCTDQCASYVANVFTTQPGGFIPSDFSFNNWFLL
TNSSTLVSGKLVTKQPLLVNCLWPVPSFEEAASTLCFEGAGFDQCNGAVLNNTVDVI
RFNLNFTTNVQSGKGATVFSLNTTGGVTLEISCYNDTVSDSSFSSYGEMPFGVTDGPR
YCYVLYNGTALKYLGTLPPSVKEIAISKWGHFYINGYNFFSTFPIDCISFNLTTGDSDV
FWTIAYTSYTEALVQVENTAITKVTYCNSYVNNIKCSQLTANLNNGFYPVSSSEVGL
VNKSVVLLPSFYTHTIVNITIGLGMKRSGYGQPIASTLSNITLPMQDNNTDVYCIRSDQ
FSVYVHSTCKSSLWDNVFKRNCTDVLDATAVIKTGTCPFSFDKLNNYLTFNKFCLSL
SPVGANCKFDVAARTRTNDQVVRSLYVIYEEGDNIVGVPSDNSGLHDLSVLHLDSCT
DYNIYGRTGVGIIRQTNRTLLSGLYYTSLSGDLLGFKNVSDGVIYSVTPCDVSAQAAV
IDGTIVGAITSINSELLGLTHWTTTPNFYYYSIYNYTNDRTRGTAIDSNDVDCEPVITY
SNIGVCKNGALVFINVTHSDGDVQPISTGNVTIPTNFTISVQVEYIQVYTTPVSIDCSR
YVCNGNPRCNKLLTQYVSACLTIEQALAMGARLENMEVDSMLFVSENALKLASVE
AFNSSETLDPIYKEWPNIGGSWLEGLKYILPSDNSKRKYRSAIEDLLFAKVVTSGLGT
VDEDYKRCTGGYDIADLVCAQYYNGIMVLPGVANADKMTMYTASLAGGITLGALG
GGAVAIPFAVAVQARLNYVALQTDVLNKNQQILASAFNQAIGNITQSFGKVNDAIHQ
TSRGLATVAKALAKVQDVVNTQGQALSHLTVQLQNNFQAISSSISDIYNRLDELSAD
AQVDRLITGRLTALNAFVSQTLTRQAEVRASRQLAKDKVNECVRSQSQRFGFCGNG
THLFSLANAAPNGMIFFHTVLLPTAYETVTAWAGICALDGDRTFGLVVKDVQLTLFR
NLDDKFYLTPRTMYQPRVATSSDFVQIEGCDVLFVNATVSDLPSIIPDYIDINQTVQDI
LENFRPNWTVPELTFDIFNATYLNLTGEIDDLEFRSEKLHNTTVELAILIDNINNTLVN
LEWLNRIETYVKWPWYVWLLIGLVVIFCIPLLLFCCCSTGCCGCIGCLGSCCHSICSR
RQFENYEPIEKVHVH
SEQ ID NO: 58-Canine Coronavirus (CCoV-HuPn-2018)
MKLLFVVLVVLPSIYGDNFPCSKFLNRTIGNHWNLIENFLLNYSIRLPPNSDVVLGDY
FPTVQPWFNCIRNNNNSLYVTMENLKALYWDYATENITSDHRQRLHVVVKGKPYSI
TVTTTRNFDAAEGAIICICKGSPPTTTTGNLDCNWGSDCRLNHKFPICPSNSQANCGN
MLYGLQWFTDEVVAYLHGAIYRISFENKWFGTVTLGDMRATTLQTAGALVDLWWF
NPVYDVTYYRVNNKNGTTIVSNCTDQCASYVDNVFTTQPGGLIPSDFSFNNWFLLTN
SSTVVSGKLVTRQPLVVNCLWPVPSFKEAASTFCFEGAGFDQCNGAVLNNTVDVIRF
NLNFTADVQSGMGATVFSLNTTGGVILEISCYNDIVSESSFYSYGDIPFGITDGPRYCY
VLYNGTTLKYLGTLPPSVKEIAISKWGHFYINGYNFFSTFPIDCISFNLTTGASGAFWT
IAYTSYTEALVQVENTAIKKVTYCNSHINNIKCSQLTANLQNGFYPVASSEVGLVNKS
VVLLPSFFAHTTVNITIDLGMKRSGYGQPIASPLSNITLPMQDNNTDVYCIRSNQFSIY
VHSTCKSSLWDNVFNQDCTDVLEATAVIKTGTCPFSFDKLNNHLTFNKFCLSLSPVG
ANCKFDVAARTRTNEQVVRSLYVIYEEGDNIVGVPSDNSGLHDLSVLHLDSCTDYNI
YGRTGVGVIRQTNSTLLSGLYYTSLSGDLLGFKNVSDGVIYSVTPCDVSAQAAVIDG
AIVGAMTSINSELLGLTHWTTTPNFYYYSIYNYTNERTRGTAIDSNDVDCEPIITYSNI
GVCKNGALVFINVTHSDGDVQPISTGNVTIPTNFTISVQVEYIQVYTTPVSIDCARYVC
NGNPRCNKLLTQYVSACQTIEQALAMGARLENMEVDSMLFVSENALKLASVEAFNS
TENLDPIYKEWPNIGGSWLGGLKDILPSHNSKRKYRSAIEDLLFDKVVTSGLGTVDE
DYKRCTGGYDIADLVCAQYYNGIMVLPGVANDDKMTMYTASLAGGITLGALGGGA
VAIPFAVAVQARLNYVALQTDVLNKNQQILANAFNQAIGNITQAFGKVNDAIHQTSK
GLATVAKVLAKVQDVVNTQGQALSHLTVQLQNNFQAISSSISDIYNRLDELSADAQV
DRLITGRLTALNAFVSQTLTRQAEVRASRQLAKDKVNECVRSQSQRFGFCGNGTHLF
SLANAAPNGMIFFHTVLLPTAYQTVTACSGICASDGDRTFGLVVKDVQLTLFRNLDD
KFYLTPRTMYQPRAATSSDFVQIEGCDVLFVNATEIDLPSIIPDYIDINQTVQDILENYR
PNWTVPELTLDIFNATYLNLTGEIDDLEFRSEKLHNTTVELAILIDNINNTLVNLEWLN
RIETYVKWPWYVWLLIGLVVVFCIPLLLFCCCSTGCCGCIGCLGSCCHSICSRRQFEN
YEPIEKVHVH
SEQ ID NO: 59-HKU16
MQRIILISTILYCARALTLADKMLDLLTFPGAHHYFRGDLQTLHSRISAESYSVNPYD
QYNYQTDSDYYINKSVHLIAPLTNLTLPISGLHRSMQPLRVGCIFGASNKIDQGFTISG
MTYPLAYCVPPFYQVTNVTYDAMRLLFAFADLNSTGDFLRINTKTMGMLNVSCSAS
PTPLGHQDADRTFYGYNKQLYCYLDTPAGMQYMGPLPANLTEITLFRTGQIYTNGF
HLGTIPSELTYVYLDKLAFQNKTVCMMANLTDTLITLNHTVIQQVTYCEKDAVQAL
ACQQSTHQLQDGFYSDPAPAVNNLPKTLVTLPKIAESSTLQINVSATYSYGSASGSIK
LSYNGSSNNSHCVQTPYFKLEQNLVCSGGCSVRIETLTCPFDLNAVSNGMSFQQFCV
STVSGQCSMQAIVNTGQPWGYVTSTLYVTYVEGQSFTGTSSDQIEDLTVLHLDQCTS
YTIYGVSGTGVITLSDLQLPHGITFRAANGELSAFKNTTTGDVYTIQPCSLPAQLAIIDS
TIVGAITSTNESYGFSNTIVTPTFYYSTNATSNCTAPKISYGELGVCADGSIGAVSQLQ
DSKPSIVPLYTGEIEIPASFKLSVQTEYLQVQTEQVVIDCPKYVCNGNPRCLQLLAQY
TSACSNIESALHSSAQLDSREITMMFQTSSQSVELANITNFQGDYNFSMILPTLPGKDR
SAIEDLLFDKVVTNGLGTVDQDYKSCSKGIAVADLVCAQYYNGIMVLPGVVDAEK
MAMYTGSLTGAMVFGGLTAAAAIPFSTAVQARLNYVALQTNVLQENQKILAESENQ
AVGNISLALSNVNTAIQQTSEALLTVSNAINKIQTVVNQQGEALAHLTAQLSQNFQAI
STSIQDIYNRLDQIQADQQVDRLITGRLAALNAYVTQLLNKLSQVRQSRILAEQKINE
CVKSQSSRYGFCGNGTHLFSLTQAAPNGIFFMHAVLVPQTFQPVVAYAGICVDGYG
YSLQPQLVLYNLNDSYRITPRNMFEPRTPTQSVFIPLTTCSVDFVNVTANNVSIIIPDY
VDVNKTVSDIINGLPNYSYPELSLDRFNHTILNLSQEIEDLQIRSQNLSATAELLQQYI
DNLNNTLVDLEWLNRVETYLKWPWYIWLLIFLAIAAFATILVTIFLCTGCCGGCFGC
CGGCFGLFSKKRRLSSEPTPVSFKLKEW
SEQ ID NO: 60-HKU19
MQCVVLVLTLISIVTSRPNSFADRVFDALTFPHASNYLHVGDKTPSRPQLLQARNQG
NYSAICPENGYITSTSYDLSKIYYLTDGDYPIDGVYKSLQPLKVTCVPEWHGNSNFND
TTGWKHYFDGRIKQNPNTIWCPCSQSGPGGAQHNAAGNSTEYIRFHSNITTSVSNLL
RLYAVDNQYYYFGCTPTPTPLTFNLTSENITLFTAEEQVHYCYANINGTVSYIGVLPP
KITELTVGRYGDIFVNGFLYFKIPNVIEYVQLSHTIPHNKQFYTVFYANMTQVLLNIS
MASINRLLYCDKDSYSSIACAVNQFEPANGFYSTSAIEKISRKFTFVTMPTVQNHSYY
SINLTIGGCGHGEYPHLSNKTGCYRTDASNINAKQITFVINTYTHDNWIQWAHKPGN
CPWALNKINNYNTAGTLQVVPENQATCCTDNQASWLYLASWTSVNVKVCFNYQPG
TTISIQPQQTGVATDISVIYENECVDYNIYGKTGTGIIQSTNVTLLAGRTYTSASGQLL
AFKYLSNQTIYSVTPCDFSNQVAVYNKSVIAAILPQNKTIFGLTNIQETPNFYIANNAH
QQQRFAMYMEPLNSQQPDCTPVLTYAQIGICADGQFVQVQPEKSQPMSTTPIVAVNI
TIPKTFNISVQTEYIQISTDNIVIDCQRYVCNGNPRCLMLLSQYQSACSTIEQALHQKA
RLESLELSTMLAYSPNTLQLANVSNFQSNNMGFNLTNLLPQNNSPQKRSVIEDLLFSK
VVTNGLGTVDVDYKKCTKGLSIADLPCAQYYNGIMVLPGVADSGLLAAYTASLTGG
MVFGGLTSAAAIPFATAVQARLNYVALQTDVLQRNQQILANAFNQAMGNITLAFKD
VKEAIATTADAIRVVAGALDKIQQVVNSQGQALSKLTGELQRNFQAISASIEDIYNRL
NDIEADAQVDRLITGRLAALNAFLTQTLTQANEVKAARELALQKINECVKDQSKRY
GFCGNGYHLFSIANAAPKGFIFFHTVLQPETTIEIQAIAGFCVSDRQTYNYYSSNMEG
QAYIARDTTQTIFLHENGTYMITPRKQYQPRTLAQADVVKISTCDVTYVNLTSIEFEQ
LIPEYVDINSTVEGILNSTLPGKIPDLNIGHYNNTILNLTTEINDLQSKAENLSMIAYQL
EEYIKNINNTLVDLEWLNRVETYLKWPWYVWLAIALAFTGFVTILITIFLCTGCCGGC
FGCCGGCFGLFSKKIDPMRQYMNRYETPTSKSDDAIPIIYKKNW
SEQ ID NO: 61-Porcine Deltacoronavirus (PDCoV)
MQRALLIMTLLSLARAKFADDLLDLLTFPGAHRFLHKPTRNSSNLYLRANNNFDVG
VLPGYPTKNVNLFSPLTNSTLPINGLHRSYQPLMLNCLTKITNHTLSMYLLPSEVQTY
SCGGAMVKYQTHDAVRIILDLIATDHISVEVVGQRGENYVFVCSEQFNYTTALHNAT
FFSLNSQLYCFTNNTYLGILPPDLTDFTVYRTGQFYANGYLLGTLPITVNYVRLYRGQ
LAANSAHFALANLTDTLITLTNTTISQITYCDKSVVDSIACQRSSHEVEDGFYSDPKSA
VRARQRTIVTLPKLPELEVVQLNISAHMDFGEARLDSVTINGNTSYCVTKPYFRLETN
FMCTGCTMNLRTDTCSFDLSAVNNGMSFSQFCLSTESGACEMKIIVTYVWNYLLRQ
RLYVTAVEGQTHTGTTSVHATDTSSVITGVCTDYTIYGVSGTGIIKPSDLLLHNGIAFT
SPTGELYAFKNITTGKTLQVLPCETPSQLIVINNTVVGAITSSNSTENNRFTTTIVTPTF
FYSTNATTFNCTKPVLSYGPISVCSDGAIVGTSTLQNTRPSIVSLYDGEIEIPSAFSLSV
QTEYLQVQAEQVIVDCPQYVCNGNSRCLQLLAQYTSACSNIEAALHSSAQLDSREIIN
MFKTSTQSLQLANITNFKGDYNFSSIITPRVGGRSAIEDLLFNKVVTSGLGTVDQDYK
SCSRDMAIADLVCSQYYNGIMVLPGVVDAEKMAMYTGSLTGAMVFGGLTAAAAIP
FATAVQARLNYVALQTNVLQENQKILAESFNQAVGNISLALSSVNDAIQQTSEALNT
VAIAIKKIQTVVNQQGEALSHLTAQLSNNFQAISTSIQDIYNRLEEVEANQQVDRLITG
RLAALNAYVTQLLNQMSQIRQSRLLAQQKINECVKSQSPRYGFCGNGTHIFSLTQTA
PNGIFFMHAVLVPNKFTRVNASAGICVDNTRGYSLQPQLILYQFNNSWRVTPRNMYE
PRLPRQADFIQLTDCSVTFYNTTAANLPNIIPDVIDVNQTVSDIIDNLPTATPPQWDVG
IYNNTILNLTVEINDLQERSKNLSQIADRLQNYIDNLNNTLVDLEWLNKVETYLKWP
WYVWLAIALALIAFVTILITIFLCTGCCGGCFGCCGGCFGLFSKKKRYTDDQPTPSFK
FKEW
SEQ ID NO: 62-human Porcine Deltacoronavirus (Hu-PDCoV)
MQRALLIMTLLCLVRAKFADDLLDLLTFPGAHRFLHKLTSNSSSFYSRANNFDVGVL
PGYPTENVNLFSPLTNSTLPINGLHRSYQPLMLNCLTKITNHTLSMYLLPSEIQTYSCG
GAMVKYQTHDAVRIILDLTVTDHISVEVVGQRGENYVFVCSEQFNYTTALHNSTFFS
LNSELYCFTNNTYLGILPPDLTDFTVYRTGQFYANGYLLGTLPITVNYVRLYRGHLSA
NSAHFALANLTDTLITLTNTTISQITYCDKSVVDSIACQRSSHEVEDGFYSDPKSAVRA
RQRTIVTLPKLPELEVVQLNISAHMDFGEARLDSVTINGNTSYCATKPYFRLETNFMC
TGCTMNLRTDTCSFDLSAVNNGMSFSQFCLSTESGACEMKIIVTYVWNYLLRQRLY
VTAVEGQTHTGTTSVHATDTSSVITDVCTDYTIYGVSGTGIIKPSDLLLHNGIAFTSPT
GELYAFKNITTGKTLQVLPCETPSQLIVINNTVVGAITSSNSTENNRFTTTIVTPTFFYS
TNATTFNCTKPVLSYGPISVCSDGAIAGTSTLQNTRPSIVSLYDGEVEIPSAFSLSVQTE
YLQVQAEQVIVDCPQYVCNGNSRCLQLLAQYTSACSNIEAALHSSAQLDSREIINMF
QTSTQSLQLANITNFKGDYNFSSILTTRLGGRSAIEDLLFNKVVTSGLGTVDQDYKAC
SRDMAIADLVCSQYYNGIMVLPGVVDAEKMAMYTGSLTGAMVFGGLTAAAAIPFA
TAVQARLNYVALQTNVLQENQKILAESFNQAVGNISLALSSVNDAIQQTSEALNTVA
IAIKKIQTVVNQQGEALSHLTAQLSNNFQAISTSIQDIYNRLEEVEANQQVDRLITGRL
AALNAYVTQLLNQMSQIRQSRLLAQQKINECVKSQSSRYGFCGNGTHIFSLTQTAPN
GIFFMHAVLVPNKFTRVNASAGICVDNIKGYSLQPQLILYQFNNSWRVTPRNMYEPR
LPRQADFIQLTDCSVTFYNTTAANLPNIIPDIIDVNQTVSDIIDNLPTATPPQWDVGIYN
NTILNLTVEINDLQERSKNLSQIADRLQNYIDNLNNTLVDLEWLNRVEIYLKWPWYI
WLAIALALIAFVTILITIFLCTGCCGGCFGCCGGCFGLFSKKKRYTDDQPTPSFKFKEW

1. Class I fusion protein or a fragment thereof, comprising at least one stabilizing mutation in the HR2 domain, wherein the protein is not a PIV-3 protein.

2. Protein or fragment according to claim 1, wherein the at least one stabilizing mutation is a mutation of an amino acid residue selected from the group consisting of S, T, A and Y at positions a and/or d of the heptad repeat motif into a hydrophobic amino acid residue.

3. Protein or fragment according to claim 2, comprising a mutation of an amino acid residue selected from the group consisting of S, T, A and Y at positions a and d of the heptad repeat motif into a hydrophobic amino acid residue.

4. Protein or fragment according to claim 1, wherein the hydrophobic amino acid residue is an amino acid selected from the group consisting of V, I, M and L.

5. Protein or fragment according to claim 1, wherein the at least one stabilizing mutation in the HR2 domain improves trimer expression and/or stabilizes the pre-fusion conformation.

6. Protein or fragment according to claim 1, wherein the class I fusion protein is a class I fusion protein with a parallel HR2 coiled-coil.

7. Protein or fragment according to claim 1, wherein the class I fusion protein is a paramyxovirus fusion (F) or a coronavirus spike(S) protein.

8. Protein or fragment according to claim 6, wherein the paramyxovirus F protein is an F protein from a virus selected from the group consisting of Nipah virus, Sendai virus, PIV-1, PIV-2, PIV-4, PIV-5, Mumps virus, Measles virus, Hendra virus, Newcastle disease virus, Avian orthoavulavirus, Canine distemper virus, Feline morbillivirus, Porcine respirovirus, Mojiang virus, Salmon aquaparamyxovirus, Cetacean morbillivirus, Reptilian ferlavirus, and Langya henipavirus.

9. Protein or fragment according to claim 7, wherein the class I fusion protein is a paramyxovirus F protein comprising a mutation of an amino acid residue selected from the group consisting of S, T, A and Y at a position corresponding to position 470 and/or position 477 in SEQ ID NO: 1 into a hydrophobic amino acid residue.

10. Protein or fragment according to claim 9, wherein the hydrophobic amino acid at position 470 and/or at position 477 is valine (V).

11. Protein or fragment according to claim 1, wherein the class I fusion protein is a coronavirus S protein comprising a mutation of an amino acid residue selected from the group consisting of S, T, A and Y at a position corresponding to position 1259 and/or position 1266 in SEQ ID NO: 53, into a hydrophobic amino acid residue.

12. Protein or fragment according to claim 11, wherein the hydrophobic amino acid at position 1259 and at position 1266 is valine (V), isoleucine (I) or leucine (L).

13. Protein or fragment according to claim 11, wherein the coronavirus S protein is from an α-coronavirus or δ-coronavirus.

14. Protein or fragment according to claim 1, wherein the fragment is a class I fusion protein ectodomain.

15. Protein or fragment according to claim 14, wherein the protein or fragment is trimeric and does not comprise a heterologous trimerization domain.

16. Protein or fragment claim 1, comprising one or more additional mutations in the head domain of the class I fusion protein.

17. Protein or fragment according to claim 16, wherein the protein is a Nipah F protein and the one or more mutations in the head domain are selected from a mutation of the amino acid residue at position 191 into P and the amino acid residue at position 452 into N, wherein the numbering of amino acid positions is according to the numbering in SEQ ID NO: 1.

18. Nucleic acid molecule encoding a protein or fragment thereof according to claim 1, preferably wherein the nucleic acid molecule is DNA or RNA.

19. (canceled)

20. Vector comprising a nucleic acid according to claim 18, wherein preferably the vector is a human recombinant adenoviral vector.

21. (canceled)

22. Vector according to claim 21, wherein the adenoviral vector is a replication-incompetent Ad26 adenoviral vector having a deletion of the E1 region and the E3 region.

23-25. (canceled)
